(19) United States
(12) Patent Application Publication
(10) Pub. No.: US 2024/0207357 A1
(43) Pub. Date: Jun. 27, 2024

(54) RECONSTITUTED HIGH DENSITY LIPOPROTEIN TREATMENT OF MYOCARDIAL INFARCTION

(71) Applicant: CSL Limited, Melbourne (AU)

(72) Inventors: Samuel Wright, Westfield, NJ (US); Charles Shear, Collegeville, PA (US); Denise D'Andrea, East Norriton, PA (US); Andreas Gille, San Marino, CA (US); Danielle Duffy, Media, PA (US)

(73) Assignee: CSL Limited, Melbourne (AU)

(21) Appl. No.: 18/230,058

(22) Filed: Aug. 3, 2023

Related U.S. Application Data
(63) Continuation of application No. 16/348,106, filed on May 7, 2019, now abandoned.
(60) Provisional application No. 62/420,050, filed on Nov. 10, 2016.
(60) Provisional application No. 62/472,240, filed on Mar. 16, 2017.

(51) Int. Cl. A61K 38/17; A61K 9/00; A61K 31/192; A61K 31/216; A61K 31/366; A61K 31/397; A61K 31/40; A61K 31/47; A61K 31/505; A61K 31/685; A61K 31/727; A61K 45/06; A61K 47/26; A61K 47/28; A61P 3/06; A61P 9/10

(52) U.S. Cl. CPC A61K 38/1709; A61K 9/0019; A61K 31/192; A61K 31/216; A61K 31/366; A61K 31/397; A61K 31/40; A61K 31/47; A61K 31/505; A61K 31/685; A61K 31/727; A61K 45/06; A61K 47/26; A61K 47/28; A61P 3/06; A61P 9/10

(57) ABSTRACT

Reconstituted HDL formulations, methods of treatment comprising same and uses thereof are provided for treating patients after an acute myocardial infarction (MI) event. The reconstituted HDL formulations comprise an apo lipoprotein, a lipid, a detergent and a stabilizer such as sucrose. Treatment of MI patients with repeated infusions of rHDL enhance cholesterol efflux capacity and do not produce significant alterations in liver or kidney function. The MI patient may have normal kidney function or moderate renal impairment.

### CROSS REFERENCE TO RELATED APPLICATIONS

This application is a continuation of U.S. application Ser. No. 16/348,106, filed May 7, 2019, which is a National Phase of PCT/AU2017/051232, filed Nov. 10, 2017, which claims priority to U.S. Provisional Applications 62/472,240 filed Mar. 16, 2017, and 62/420,050 filed Nov. 10, 2016.

### TECHNICAL FIELD

THIS INVENTION relates to treatment of acute myocardial infarction. More particularly, this invention relates to the use of a particular low toxicity reconstituted high density lipoprotein formulation for treating acute myocardial infarction. Also described is the use of such a formulation for treating patients who have not previously or recently experienced an acute myocardial infarction (MI) event, to reduce the risk of a major adverse cardiovascular event (MACE) in such patients.

### BACKGROUND

Despite advances in therapeutic strategies for acute myocardial infarction (MI), patients remain at a high risk for recurrent ischemic events, particularly in the immediate weeks to months following the event 1. Recurrent events are most commonly due to additional plaque rupture or erosion, and are associated with significant morbidity and mortality^{2, 3}. While they may occur at the site of the index MI vessel, they are equally likely to occur at a different site anywhere in the coronary artery tree². Although a low level of high density lipoprotein cholesterol (HDL-C) is a risk factor for major adverse cardiovascular events (MACE)⁴⁻¹², it remains unclear if raising HDL will reduce MACE as several therapies that raised HDL-C were not associated with improved clinical outcomes¹³⁻¹⁷. These studies may have been limited by the failure to enrich for patients with high modifiable risk, off target toxicity, or failure to raise functional HDL. Cholesterol efflux capacity (CEC), an ex-vivo measure of HDL function, evaluates the ability of HDL to remove excess cholesterol from atherosclerotic plaque for transport to the liver. CEC is a correlate of MACE that is independent of HDL-C, and it may be more viable to improve clinical outcomes by identifying pharmacotherapies that act rapidly following acute MI to improve cholesterol efflux and thereby reduce plaque burden and stabilize vulnerable plaque, rather than therapies that raise HDL alone¹⁸⁻²⁰. Importantly, the majority of the failed HDL-C raising trials evaluated chronic pharmacotherapy, and therapy was not initiated in the immediate post-myocardial infarction (MI) period, a time when cholesterol efflux is significantly impaired²¹⁻²³.

### SUMMARY

The invention is broadly directed to the use of reconstituted HDL (rHDL) formulations to treat patients after an acute myocardial infarction (MI) event. In a particular form, the invention provides treatment of MI patients with repeated infusions of rHDL that enhance cholesterol efflux capacity and do not produce significant alterations in liver or kidney function. In some embodiments, the MI patient has normal kidney function. In some embodiments, the MI patient has mild renal impairment. In some embodiments the MI patient has moderate renal impairment. The invention is also broadly directed to the use of rHDL formulations for reducing the risk of a major adverse cardiovascular event (MACE) in patients who have not previously experienced an MI event, or who have not recently experienced an MI event (i.e., who have not experienced an MI event within seven days prior to starting treatment). In a particular embodiment, such patients have moderate renal impairment. In some embodiments, such patients have mild renal impairment. In some embodiments, such patients have normal kidney function. The treatment of patients who have not previously or recently had an MI event may be with repeated infusions of rHDL, may enhance cholesterol efflux capacity, and in preferred embodiments does not produce substantial alterations in liver or kidney function.

An aspect of the invention provides a method for increasing cholesterol efflux capacity (CEC) in a human patient after an acute myocardial infarction (MI) event, including the step of:
within about seven (7) days of the acute MI event, administering to the patient a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol); and
subsequently administering the rHDL formulation to the patient, preferably for at least about four (4) weeks;
thereby increasing cholesterol efflux capacity (CEC) without causing a substantial alteration in liver or kidney function of the human.

Suitably, the dose within about seven (7) days of the acute MI event, is an initial dose of the reconstituted high density lipoprotein (rHDL) formulation. Subsequently, the patient is administered at least three (3) further doses of the rHDL formulation, for a total of at least four doses (including the initial dose) preferably over at least about four (4) weeks from and including the initial dose. The treatment period may be defined as the time from the administration of the initial dose of rHDL until one week following the final administered dose.

A related aspect of the invention provides a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol) for use in increasing cholesterol efflux capacity (CEC) in a human patient after an acute myocardial infarction (MI) event wherein the rHDL formulation is administered to the human patient within about seven (7) days of the acute MI event and then subsequently administered to the patient, preferably for at least about four (4) weeks.

Another aspect of the invention provides a method for treating an acute myocardial infarction (MI) event in a human patient, including the steps of:
within about seven (7) days of the acute MI event, administering to the patient a reconstituted high density lipoprotein (rHDL) formulation an apolipoprotein or a fragment thereof, a lipid, a stabilizer and optionally a detergent, wherein the ratio between the apolipoprotein and the lipid is from about 1:20 to about 1:120 (mol:mol); and
subsequently administering the rHDL formulation to the patient, preferably for at least about four (4) weeks;
thereby treating the acute myocardial infarction (MI) event in the patient without causing a substantial alteration in liver or kidney function of the patient.

Suitably, the dose within about seven (7) days of the acute MI event, is an initial dose of the reconstituted high density lipoprotein (rHDL) formulation. Subsequently, the patient is administered at least three (3) further doses of the rHDL formulation, for a total of at least four doses (including the initial dose) preferably over at least about four (4) weeks from and including the initial dose. The treatment period may be defined as the time from the administration of the initial dose of rHDL until one week following the final administered dose.

A related aspect of the invention provides a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol) for use in treating an acute myocardial infarction (MI) event in a human patient, wherein the rHDL formulation is administered to the human patient within about seven (7) days of the acute MI event and then subsequently administered to the patient, preferably for at least about four (4) weeks.

Another aspect of the invention provides a method for reducing the risk of a major adverse cardiac event (MACE) in a human patient who has not previously experienced an MI event, or who has not experienced an MI event within seven days prior to starting treatment, including the step of:
administering to the patient a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol),
thereby reducing the risk of a MACE in the patient, and in some embodiments without causing a substantial alteration in liver or kidney function of the patient.

A related aspect of the invention provides a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol) for use in method of reducing the risk of a MACE in a human patient who has not previously experienced an MI event, or has not experienced an MI event within seven days prior to starting treatment, and in some embodiments without causing a substantial alteration in liver or kidney function of the patient.

Another aspect of the invention provides a method for increasing CEC in a human patient who has not previously experienced an MI event, or has not experienced an MI event within seven days prior to starting treatment, including the step of:
administering to the patient a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol),
thereby increasing cholesterol efflux capacity (CEC), and in some embodiments without causing a substantial alteration in liver or kidney function of the human.

A related aspect of the invention provides a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol) for use in method of increasing cholesterol efflux capacity (CEC) in a human patient who has not previously experienced an MI event, or has not experienced an MI event within seven days prior to starting treatment, and in some embodiments without causing a substantial alteration in liver or kidney function of the human.

In embodiments where the patient has not previously experienced an MI event, or has not experienced an MI event within seven days prior to starting treatment, the patient may have normal renal function, moderate renal impairment, or may have mild renal impairment. In particular embodiments, the patient has moderate renal function, as in Example 2.

Preferably, the methods described herein increase cholesterol efflux capacity (CEC) in the human.

In some embodiments of the aforementioned aspects, total CEC is increased in the range 1.5-fold to 2.5 fold.

In some embodiments of the aforementioned aspects, ABCA1-dependent CEC is increased in the range about 3-fold to about 5-fold.

Suitably, according to the aforementioned aspects, where the patient has recently experienced an acute MI event, the patient is initially administered rHDL within 5 days of the acute MI event. In some embodiments, the human patient is initially administered the rHDL formulation no earlier than 12 hours after the acute MI event or after administration of a contrast agent for angiography.

Preferably, subsequent administration of the rHDL formulation is weekly, preferably for at least four (4) weeks.

Where the patient has not previously experienced an MI event, or has not experienced an MI event within seven days prior to starting treatment, the initial administration of the rHDL formulation may be at any time, and may be followed by subsequent administrations at suitable time points, such as over a period of 1, 2, 3 or 4 weeks, or longer. Preferably, subsequent administration of the rHDL formulation is weekly, preferably for four (4) weeks, or longer.

Suitably, according to the aforementioned aspects the rHDL formulation is intravenously (IV) infused.

Suitably, the apolipoprotein is Apo AI. Preferably, the amount of Apo AI in the rHDL formulation is at least 2 g or at least 4 g or at least 6 g. In a particular embodiment the amount of Apo AI in the rHDL formulation is from 2 g to 8 g. In an embodiment the amount of Apo AI in the rHDL formulation is 6 g.

Suitably, the stabilizer is sucrose. Preferably, the sucrose is present in the rHDL formulation at a concentration of about 1.0% to less than 6.0% w/w.

In a particular embodiment there is provided a method for increasing cholesterol efflux capacity (CEC) in a human patient after an acute myocardial infarction (MI) event, including the steps of: within about seven (7) days of the acute MI event, administering to the patient a reconstituted high density lipoprotein (rHDL) formulation comprising at least 6 g of an apoA-I, phosphatidylcholine, a stabilizer and sodium cholate at a level selected from the group consisting of about 0.5-1.5 g/L and/or about 0.010-0.030 g/g apoA-I, and from about 1.0% to less than 6.0% w/w of sucrose, wherein the ratio between the apoA-I and the phosphatidylcholine is from about 1:20 to about 1:120 (mol:mol); and subsequently administering the rHDL formulation to the human, for at least four (4) weeks; thereby increasing cholesterol efflux capacity (CEC) in the human patient without causing a substantial alteration in liver and/or kidney function of the human, wherein a substantial alteration in liver function is an ALT of more than about 2 or 3 times the upper limit of normal (ULN); or an increase in total bilirubin of at least 1.5 to 2 times ULN; and the substantial alteration in kidney function is a serum creatinine greater than or equal to about 1.2-1.5 times the baseline value and/or an eGFR substantially less than 90 mL/min/m2 (e.g. substantially less than 90 mL/min/1.73 m²). For example, a substantial alteration in kidney function may be indicated by an eGFR substantially less than 90 mL/min/1.73 m². Additionally or alternatively, a patient may be considered to not have a substantial alteration of kidney function wherein the eGFR after rHDL treatment is within 30, 20 or 10 mL/min/1.73 m² of the eGFR before treatment, as discussed in more detail below.

In a related particular embodiment, there is provided a reconstituted high density lipoprotein (rHDL) formulation comprising at least 6 g of an apoA-I, phosphatidylcholine, a stabilizer and sodium cholate at a level selected from the group consisting of about 0.5-1.5 g/L and/or about 0.010-0.030 g/g apoA-I, and from about 1.0% to less than 6.0% w/w of sucrose, wherein the ratio between the apoA-I and the phosphatidylcholine is from about 1:20 to about 1:120 (mol:mol), for use in increasing cholesterol efflux capacity (CEC) in a human patient within about seven (7) days of an acute MI event, wherein the rHDL formulation is subsequently administered to the human patient for at least about four (4) weeks, thereby increasing cholesterol efflux capacity (CEC) in the human patient without causing a substantial alteration in liver and/or kidney function of the human; wherein a substantial alteration in liver function is an ALT of more than about 2 or 3 times the upper limit of normal (ULN); or an increase in total bilirubin of at least 1.5 to 2 times ULN; and the substantial alteration in kidney function is a serum creatinine greater than or equal to about 1.2-1.5 times the baseline value and/or an eGFR substantially less than 90 mL/min/m2 (e.g. substantially less than 90 mL/min/1.73 m²). For example, a substantial alteration in kidney function may be indicated by an eGFR substantially less than 90 mL/min/1.73 m²). Additionally or alternatively, a patient may be considered to not have a substantial alteration of kidney function wherein the eGFR after rHDL treatment is within 30, 20 or 10 mL/min/1.73 m² of the eGFR before treatment, as discussed in more detail below.

In a further embodiment there is provided a method for reducing the risk of a MACE and/or increasing CEC in a human patient who has not previously experienced an MI event, or has not experienced an MI event within seven days prior to starting treatment, including the steps of: administering to the patient a reconstituted high density lipoprotein (rHDL) formulation comprising at least 6 g of an apoA-I, phosphatidylcholine, a stabilizer and sodium cholate at a level selected from the group consisting of about 0.5-1.5 g/L and/or about 0.010-0.030 g/g apoA-I, and from about 1.0% to less than 6.0% w/w of sucrose, wherein the ratio between the apoA-I and the phosphatidylcholine is from about 1:20 to about 1:120 (mol:mol) thereby reducing the risk of a MACE and/or increasing CEC in the patient. In some embodiments, this reduction in the risk of a MACE and/or increase in CEC in the patient occurs without causing a substantial alteration in liver and/or kidney function of the human.

In a related particular embodiment, there is provided a reconstituted high density lipoprotein (rHDL) formulation comprising at least 6 g of an apoA-I, phosphatidylcholine, a stabilizer and sodium cholate at a level selected from the group consisting of about 0.5-1.5 g/L and/or about 0.010-0.030 g/g apoA-I, and from about 1.0% to less than 6.0% w/w of sucrose, wherein the ratio between the apoA-I and the phosphatidylcholine is from about 1:20 to about 1:120 (mol:mol), for use in method of reducing the risk of a MACE and/or increasing CEC in a human patient who has not previously experienced an MI event, or has not experienced an MI event within seven days prior to starting treatment. In some embodiments, this reduction in the risk of a MACE and/or increase in CEC in the patient occurs without causing a substantial alteration in liver and/or kidney function of the human.

It will also be appreciated that the method disclosed herein may include the administration of one or more additional therapeutic agents. Likewise the reconstituted high density lipoprotein (rHDL) formulation as disclosed herein for use in the specific methods as disclosed herein may be used with one or more additional therapeutic agents. Suitably, the one or more additional therapeutic agents may assist or facilitate treatment, prevention or reduction in risk of an acute myocardial infarction (MI) event and/or MACE and/or increasing cholesterol efflux capacity (CEC) in a human patient, although without limitation thereto.

Where the reconstituted high density lipoprotein (rHDL) formulation as disclosed herein is used or is for use in a particular method as specified herein with one or more additional therapeutic agents, this can be described as a rHDL formulation as referred to herein for use in that method, in combination with the one or more additional therapeutic agent (e.g. one or more lipid-modifying agents; one or more cholesterol absorption inhibitors; one or more anti-coagulants; one or more anti-hypertensive agents; and one or more bile acid binding molecules). This can also be described as one or more therapeutic agent selected from one or more lipid-modifying agents; one or more cholesterol absorption inhibitors; one or more anti-coagulants; one or more anti-hypertensive agents; and one or more bile acid binding molecules for use in that method, in combination with a rHDL formulation as referred to herein. A rHDL formulation as referred to herein and one or more additional therapeutic agent (e.g. one or more lipid-modifying agents; one or more cholesterol absorption inhibitors; one or more anti-coagulants; one or more anti-hypertensive agents; and one or more bile acid binding molecules) for use as a combined preparation in a particular method as specified herein is also provided. The agents of the combined preparation may be for simultaneous or sequential use.

The one or more additional therapeutic agents may include: one or more lipid-modifying agents; one or more cholesterol absorption inhibitors; one or more anti-coagulants; one or more anti-hypertensive agents; and one or more bile acid binding molecules.

Throughout this specification, unless otherwise indicated, “comprise”, “comprises” and “comprising” are used inclusively rather than exclusively, so that a stated integer or group of integers may include one or more other non-stated integers or groups of integers.

It will also be appreciated that the indefinite articles “a” and “an” are not to be read as singular or as otherwise excluding more than one or more than a single subject to which the indefinite article refers. For example, “a” protein includes one protein, one or more proteins or a plurality of proteins.

As used herein, a human patient “who has not recently experienced an MI event” refers to a patient has not experienced an MI event within seven days prior to starting treating. That is, at the time of the first administration of the rHDL formulation as described herein, it has been eight days or more since the patient experienced an MI event. In some embodiments, such a patient has not experienced an MI event within 8, 9 or 10 days, or more, such as 2, 3, or 4 weeks, 1, 2, 3, 4, 5, 6, 7, 8, 9, 10, 11, or 12 months, or 1, 2, 3, 4, 5, 10, 15, 20, 30, 40, 50, 60, 70, 80, or 90 years prior to starting treatment. Additionally or alternatively, in some embodiments, such patients have not been diagnosed with an MI event that occurred in one of the periods of time referred to above.

As noted above, as used herein “a substantial alteration in liver function” refers to an ALT of more than about 2 or 3 times the upper limit of normal (ULN); or an increase in total bilirubin of at least 1.5 to 2 times ULN, and is used interchangeably with the phrase “a significant alteration in liver function.”

As noted above, as used herein “a substantial alteration in kidney function” refers to a serum creatinine greater than or equal to about 1.2-1.5 times the baseline value and/or an eGFR substantially less than 90 mL/min/m² (e.g. substantially less than 90 mL/min/1.73 m²). For example, a substantial alteration in kidney function may be indicated by an eGFR substantially less than 90 mL/min/1.73 m²). Additionally or alternatively, a patient may be considered to not have a substantial alteration of kidney function wherein the eGFR after rHDL treatment is within 30, 20 or 10 mL/min/1.73 m² of the eGFR before treatment, as discussed in more detail below. As used herein “a substantial alteration in kidney function” is used interchangeably with the phrase “a significant alteration in liver function.”

### BRIEF DESCRIPTION OF THE FIGURES

FIG. 1: Consort diagram.

FIG. 2: Time-to-occurrence of first MACE. Composite of CV death, non-fatal MI, ischemic stroke, and hospitalization for unstable angina. The dotted line at Day 112 indicates the final end of study visit.

FIG. 3: Time-to-occurrence of first Exploratory MACE. Composite of CV death, non-fatal MI, and stroke. The dotted line at Day 112 indicates the final end of study visit.

FIG. 4: Days from Randomization until Death.

FIG. 5: ApoA-I profiles after infusion with CSL112 in subjects with moderate renal impairment (Mod RI) or normal renal function (NRF). Values shown are mean (baseline-corrected) along with standard-deviation.

FIGS. 6A-6B: Cholesterol efflux capacities (CEC) and pre-β1-HDL levels after infusion with CSL 112 in subjects with moderate renal impairment (Mod RI) or normal renal function (NRF). Values shown are mean (baseline-corrected) along with standard-deviation.

FIGS. 7A-7B: The effects of increasing the dosage of CSL112 on cholesterol efflux capacities (CEC) and pre-β1-HDL levels in subjects with moderate renal impairment (Mod RI) or normal renal function (NRF). Shown are the individual data points alongside the regression lines.

FIG. 8: Conversion of unesterified cholesterol (HDL-UC) to esterified cholesterol (HDL-EC) following infusion with CSL112 in subjects with moderate renal impairment (Mod RI) and normal renal function (NRF). Values shown are mean (baseline-corrected) along with standard-deviation for 6 g of CSL112.

FIG. 9: Subject Disposition. Subjects were considered to have completed the study if they completed all scheduled study visits up to and including the Safety Follow-up Period/Visit 8.

FIG. 10: Aggregate Box Plots of AEGIS-I and 2001 Serum Creatinine Change from Baseline Values (Central Laboratory) by Renal Function, Visit and Treatment (Safety Population). eGFR=estimated Glomerular Filtration Rate. Note: The ends of each box represent the upper and lower quartiles, the median is marked by a horizontal line inside the box, whilst the circles (CSL112) and squares (Placebo) represent the mean values. Two vertical whiskers extend from the lower and upper quartiles to the smallest and largest non-outlier values respectively. Outliers are presented as individual data points beyond the ends of each whisker. In order to better identify trends, the Y-axis has been truncated and as a result extreme values are not presented. Study CSL112-2001 Visit 7, Day 29 (7 to 10 days after last infusion) includes data for subjects who discontinued study treatment or who withdrew from the study early. Subjects with Severe Renal Impairment (eGFR <30 mL/min/1.73 m²) are excluded from the aggregate analyses. Scheduled Study Day [X]: AEGIS-I Visit/2001 Visit—Day 2: 2a/3, Day 8: 3/4, Day 15: 4/5, Day 22: 5/6, Day 29: 6/7

FIGS. 11A-11C: Aggregate Box Plots of AEGIS-I and 2001 Serum Creatinine Change from Baseline Values (Central Laboratory) by Time Between Angiography and First Dose, Renal Function, Visit and Treatment (Safety Population). A: Subgroup: 12-<24 Hours; B: Subgroup: 24-<48 Hours; C: Subgroup: >=48 Hours. eGFR=estimated Glomerular Filtration Rate. Note: The ends of each box represent the upper and lower quartiles, the median is marked by a horizontal line inside the box, whilst the circles (CSL112) and squares (Placebo) represent the mean values. Two vertical whiskers extend from the lower and upper quartiles to the smallest and largest non-outlier values respectively. Outliers are presented as individual data points beyond the ends of each whisker. In order to better identify trends, the Y-axis has been truncated and as a result extreme values are not presented. Study CSL112_2001 Visit 7, Day 29 (7 to 10 days after last infusion) includes data for subjects who discontinued study treatment or who withdrew from the study early. Subjects with Severe Renal Impairment (eGFR <30 mL/min/1.73 m²) are excluded from the aggregate analyses. Scheduled Study Day [X]: AEGIS-I Visit/2001 Visit—Day 2: 2a/3, Day 8: 3/4, Day 15: 4/5, Day 22: 5/6, Day 29: 6/7.

FIG. 12: Aggregate Box Plots of AEGIS-I and 2001 eGFR Change from Baseline Values (Central Laboratory) by Renal Function, Visit and Treatment (Safety Population) eGFR=estimated Glomerular Filtration Rate. Note: The ends of each box represent the upper and lower quartiles, the median is marked by a horizontal line inside the box, whilst the circles (CSL112) and squares (Placebo) represent the mean values. Two vertical whiskers extend from the lower and upper quartiles to the smallest and largest non-outlier values respectively. Outliers are presented as individual data points beyond the ends of each whisker. Study CSL112_2001 Visit 7, Day 29 (7 to 10 days after last infusion) includes data for subjects who discontinued study treatment or who withdrew from the study early. Subjects with Severe Renal Impairment (eGFR <30 mL/min/1.73 m²) are excluded from the aggregate analyses. Scheduled Study Day [X]: AEGIS-I Visit/2001 Visit—Day 2: 2a/3, Day 8: 3/4, Day 15: 4/5, Day 22: 5/6, Day 29: 6/7.

FIG. 13: Total cholesterol efflux capacity, CEC (%) in the patient population receiving CSL112 (6 g) from CSL112_2001 (Example 3) to patients receiving CSL112 from AEGIS-I (Example 1) at baseline, visit 2, 3 and 6.

FIG. 14: Cholesterol ABCA1 independent CEC efflux capacity (%) in the patient population receiving CSL112 (6 g) from CSL112_2001 (Example 3) to patients receiving CSL112 from AEGIS-I (Example 1) at baseline, visit 2, 3 and 6.

FIG. 15: Cholesterol ABCA1 dependent CEC efflux capacity (%) in the patient population receiving CSL112 (6 g) from CSL112_2001 (Example 3) to patients receiving CSL112 from AEGIS-I (Example 1) at baseline, visit 2, 3 and 6.

### DETAILED DESCRIPTION

In some aspects, the invention is predicated on the discovery that administration of reconstituted HDL (rHDL) formulations may be useful in treating acute MI patients. More particularly, four (4) weekly infusions of rHDL formulations such as CSL112 are efficacious, well tolerated and are not associated with any significant alterations in liver or kidney function or other safety concern. Formulations such as CSL112 enhance cholesterol efflux (CEC) after administration to patients. This effect has been shown for acute MI patients with normal renal function and mild renal impairment (see Example 1).

In some aspects, the invention relates to the discovery that administration of reconstituted HDL (rHDL) formulations to patients with moderate renal impairment (Mod RI) enhances cholesterol efflux (CEC). Similar effects on CEC were observed in healthy and moderate renal impairment patients to those results shown in Example 1, following the administration of rHDL formulations. In addition, the increase in pre-β1-HDL was greater for the patients with moderate renal impairment (Mod RI) than it is for those with normal renal function (see Example 2). These results were obtain in Mod RI subjects who had not experienced an MI event within seven days prior to starting treatment. Thus, in some aspects, the invention relates to the discovery that administration of reconstituted HDL (rHDL) formulations to patients who have not previously experienced an MI event, or who have not recently experienced an MI event, enhances cholesterol efflux (CEC), and so may be useful to reduce the risk of a MACE. Such subjects may have moderate renal impairment, mild renal impairment, or normal kidney function. In further embodiments, data presented in Example 3 show the safety and efficacy of administration of rHDL to subjects with Mod RI, these patients representing an important high risk subset of MI patients with a significant unmet medical need.

While not wanting to be bound by theory, the clinical significance of the results achieved in Mod RI patients is twofold. Firstly it confirms that the effect of rHDL on CEC in acute MI patients can be replicated in Mod RI patients. In addition, the fact that increases in CEC were observed following rHDL administration in patients who were not acute MI patients supports the use of rHDL to reduce the risk of a MACE, based on its ability to increase CEC.

As disclosed herein, in certain aspects the invention provides treatment of human patients after an acute MI event. MI is typically the result of coronary heart disease (CHD), or related diseases, disorders or conditions including coronary artery disease, ischemic heart disease, atherosclerosis, angina, ventricular arrhythmia and/or ventricular fibrillation. CHD results from the gradual build-up of cholesterol in the coronary arteries that may result in myocardial infarction (MI), a potentially fatal destruction of heart muscle.

Acute coronary syndrome (ACS) refers to a spectrum of clinical presentations ranging from those for ST-segment elevation myocardial infarction (STEMI) to presentations found in non-ST-segment elevation myocardial infarction (NSTEMI) or in unstable angina (UA). It is almost always associated with rupture or erosion of an atherosclerotic plaque and partial or complete thrombosis of the infarct-related artery.

As generally used herein “major adverse cardiac event” or “MACE” includes cardiovascular death, fatal or non-fatal MI, UA, fatal or non-fatal stroke, need for a revascularization procedure, heart failure, resuscitated cardiac arrest, and/or new objective evidence of ischemia, as well as any and all subcategories of events falling within each of these event types (e.g., STEMI and NSTEMI, documented UA requiring urgent hospitalization). In certain embodiments, the MACE is cardiovascular death, fatal or non-fatal MI, UA (including UA requiring urgent hospitalization), fatal or non-fatal stroke, and/or risk of or danger associated with revascularization. In certain embodiments, the MACE is cardiovascular death, fatal or non-fatal MI, and ischemic stroke. In certain embodiments, the MACE is cardiovascular death, fatal or non-fatal MI, e.g. MI. In certain embodiments, treating or preventing coronary heart disease (or reducing the risks of coronary heart disease, or treating patients who are at risk of MACE, including patients who have had an acute MI or patients who have not had an acute MI, or who have not experienced an MI event within seven days prior to starting treatment) with a formulation such as rHDL reduces the likelihood of occurrence of a MACE, delays the occurrence of a MACE, and/or decreases the severity of a MACE. For each of these, the effect on MACEs may refer to an effect on MACEs generally (e.g., a reduction in the likelihood of occurrence of all types of MACE), an effect on one or more specific types of MACE e.g. a reduction in the likelihood of death, non-fatal MI, UA requiring urgent hospitalization, non-fatal stroke, or need for or risk relating to a revascularization procedure, or a combination thereof.

In accordance with some aspects described herein, the rHDL formulation is for use in either (i) reducing the risk of a further MACE in a patient who has recently experienced a MI (i.e., who has experienced an MI within seven days prior to starting treatment) or (ii) reducing the risk of a MACE in a patient who has not experienced a MI, or who has not recently experienced an MI event (i.e., who has not experienced an MI event within seven days prior to starting treatment). In these contexts, reducing the risk of a MACE can mean reducing the likelihood of occurrence of a MACE, delaying the occurrence of a MACE, and/or decreasing the severity of a MACE. This may occur by increasing CEC; thus, in preferred embodiments the reduction in risk of MACE (or risk of further MACE) is accompanied by an increase in CEC, more preferably an increase in ABCA1-dependent CEC.

Patients who are at risk of a MACE include patients who have experienced a MI, and patients with coronary heart disease or related diseases as set out above. Such patients are particularly envisaged as subjects in the present invention.

The term “myocardial infarction” (also termed an “acute myocardial infarction,” “acute MI” or “AMI”) is well understood in the art and is synonymous with the more commonly used term “heart attack”. Acute MI occurs when blood flow stops to a part of the heart causing damage to the heart muscle. Acute MI may cause heart failure, an irregular heartbeat (including serious types), cardiogenic shock, or cardiac arrest.

The predominant cause of acute MI is coronary artery disease and acute MI often arises through the blockage of a coronary artery caused by a rupture of an atherosclerotic plaque. Risk factors include high blood pressure, smoking, diabetes, lack of exercise, obesity, high blood cholesterol, poor diet, and excessive alcohol intake.

Acute MIs are commonly diagnosed by electrocardiograms (ECGs, which can determine whether the acute MI is a ST-segment elevation myocardial infarction (STEMI) or a non-ST-segment elevation myocardial infarction (NSTEMI)), blood tests (e.g. to detect troponin) and coronary angiogram. An acute MI patient may therefore have experienced a STEMI or a NSTEMI. Recognised criteria for determining acute MI are set out e.g. in Thygesen et al.³⁰.

Without being bound by theory, the increase in CEC that results from the administration of rHDL (as shown in the examples) is believed to be associated with efflux of cholesterol from atherosclerotic plaques, and a consequent reduction in the likelihood of a MACE.

As used herein, “treating” or “treat” or “treatment” refers to a therapeutic intervention that at least party eliminates or ameliorates one or more existing or previously identified pathologies or symptoms of a disease or condition. In some embodiments, treatment after an acute MI event may at least partly or temporarily prevent or suppress, or reduce the likelihood of a further MI event.

It will be appreciated that treatment may be considered to have occurred even where some symptoms of the disease or condition appear or persist and does not require complete or absolute elimination, amelioration, prevention or suppression of the disease, condition or symptom.

A “reduction” or “increase” in any parameter, as referred to herein, is typically by any amount but is preferably by a statistically significant amount, and is with reference to that parameter in the absence of the treatment that is referred to. For example, a reduction in the risk of a MACE (e.g. a reduction in the likelihood of occurrence or a decrease in the severity of a MACE) is a reduction in the risk of MACE when compared to the risk of MACE (e.g. likelihood of occurrence or the severity of a MACE) in the absence of the treatment described herein. This reduction or decrease may be by any amount (e.g., 5, 10, 15, 20, 25, 50%, or greater). Likewise, where the reduction in risk is manifest as a delay in the occurrence of a MACE, this delay is with reference to the timing of the MACE in the absence of the treatment described herein, and may be by any amount (e.g. a delay of 1, 2, 3, 4, 5, or 6 months, or longer, or 1, 2, 5, or 10 years, or longer, e.g., 1 month to 10 years) but is preferably a statistically significant delay.

In certain aspects of the invention the human patient is treated within 7 days of an acute MI event. In other aspects the human patient has not had an MI event, or has not recently had an MI event, i.e., has not experienced an MI event within seven days prior to starting treatment (i.e., at the time of starting treatment it has been longer than seven days since the patient had an MI event). As discussed above, MI diagnosis is routine. In certain embodiments the human patient has not experienced an MI event within a period of 8, 9, or 10 days or more prior to starting treatment, or 2, 3, or 4 weeks prior to starting treatment, or longer, or within a period of 1, 2, 3, 4, 5, 6, 7, 8, 9, 10, 11, or 12 months prior to starting treatment, or longer, or within a period of 1, 2, 5, 10, 15, 20, 30, 40, 50, 60, 70, 80, 90 years prior to starting treatment. Alternatively, the human patient has not been diagnosed with an MI event that occurred in one of the periods of time referred to above.

The patient may be at risk of a MACE for any reason, such as because they suffer from coronary heart disease, ischemic heart disease, atherosclerosis, angina, ventricular arrhythmia and/or ventricular fibrillation, or they may have had an acute MI (including having an acute MI with in the last 7 days). Alternatively or additionally, the patient may have one or more other risk factors for a MACE, e.g. they may:
be age 45 or older (e.g., at least 50, 55, 60, 65, 70, 75, 80, or 85);
smoke;
have high blood pressure (140/90 mmHg or higher);
have high blood cholesterol or triglyceride levels, e.g. high low-density lipoprotein (LDL) cholesterol (fasting LDL-cholesterol levels of 160 to 199 mg/dL or 4.1 to 4.9 mmol/L) or high triglyceride levels;
have diabetes;
have a family history of MI;
be physically inactive;
be obese (e.g. a BMI of 30 or more).

The human patients to be treated may have any status with respect to their renal function. Preferred examples include patients with normal renal function, mild renal impairment and moderate renal impairment. Renal impairment is a prevalent concurrent condition in acute coronary syndrome, with approximately 30% of subjects having stage 3 chronic kidney disease. Kidney function is routinely determined using the Chronic Kidney Disease Epidemiology Collaboration Equation (see, e.g., Levey, 2009 Ann Intern Med May 5; 150(9): 604-612), giving a value of estimated glomerular filtration rate (eGFR) which is correlated with renal function status (see, e.g., Kidney Disease: Improving Global Outcomes (KDIGO) CKD Work Group. KDIGO 2012 Clinical Practice Guideline for the Evaluation and Management of Chronic Kidney Disease. Kidney inter., Suppl. 2013; 3: 1-150). The glomerular filtration rate (GFR) is considered to be the best overall index of kidney function in health and disease. Normal renal function (Kidney Function Stage 1) is generally defined as an eGFR of >90 mL/min/1.73 m². Patients with mild renal impairment (Kidney Function Stage 2) have an eGFR of ≥60 to <90 mL/min/1.73 m² and patients with moderate renal impairment have an eGFR of ≥30 to <60 mL/min/1.73 m². Patients with moderate renal impairment may be further classified into patients having an eGFR of ≥45 to <60 mL/min/1.73 m² (Kidney Function Stage 3a) and patients having an eGFR of ≥30 to <45 mL/min/1.73 m² (Kidney Function Stage 3b). Patients with severe renal impairment have an eGFR of ≥15 to <30 mL/min/1.73 m² (Kidney Function Stage 4), while patients having an eGFR of <15 mL/min/1.73 m² (Kidney Function Stage 5) are considered to be in kidney failure.

As noted elsewhere, in preferred embodiments, the rHDL treatment does not cause a substantial alteration in kidney function, but patients who have renal impairment, e.g. mild or moderate renal impairment before rHDL treatment commences, may be treated in accordance with the invention.

In some embodiments the human patient who is treated within 7 days of an acute myocardial event has normal renal function, mild renal impairment, or moderate renal impairment.

In some embodiments, the human patient who has not previously experienced an MI event, or has not recently experienced an MI event (i.e., not experienced an MI event within seven days prior to starting treatment) has moderate renal impairment. In other embodiments, such patient have mild renal impairment. In other embodiments, such patients have normal kidney function. In particular embodiments, the treatment is of patients with moderate renal impairment, as illustrated in Example 2 and Example 3.

Within the context of the present invention, the term “reconstituted HDL (rHDL) formulation” means any artificially-produced lipoprotein formulation or composition that is functionally similar to, analogous to, corresponds to, or mimics, high density lipoprotein (HDL), typically present in blood plasma. rHDL formulations include within their scope “HDL mimetics” and “synthetic HDL particles”. The rHDL formulation suitably comprises an apolipoprotein, a lipid, a stabilizer and optionally a detergent. Particular embodiments of rHDL formulations will be discussed in more detail hereinafter. A particularly preferred embodiment of an rHDL formulation is referred to herein as “CSL112”. Reference is made to International Publications WO2012/000048, WO2013/090978 and WO2014/066943 which provide particular examples of CSL112 formulations.

Suitably, the methods of treatment of the aforementioned aspects (e.g. wherein the patient is treated within about 7 days of an acute myocardial event) include administration of an initial dose of an rHDL formulation to a human patient within about seven (7) days of an acute MI event. This may include initial administration a few hours (e.g. 4, 6, 12 or 18 hrs) after the acute MI event, or 1, 2, 3, 4, 5, 6 or 7 days (or any hourly period between these) after the acute MI event. Preferably, the treatment includes administration of an initial dose of an rHDL formulation to a human patient within about five (5) days of an acute MI event.

Where the patient is not treated within 7 days of an acute MI (e.g. because the patient has not had a MI, or has not recently had an MI), the initial dose may be administered at any suitable time.

In a particular embodiment, the human patient may have been administered a contrast agent for angiography. In such an embodiment, an initial dose of rHDL formulation occurs no earlier than 12 hours after administration of the contrast agent.

The same or different dosage of rHDL formulation may subsequently be administered to the human patient one or more times per week for about 2, 3, 4, 5, 6, 7, 8, 9 or 10 weeks. In a preferred form, the same dosage of rHDL formulation is subsequently administered to the human patient once weekly for about 4 weeks. The treatment period may be defined as the time from the administration of the initial dose of rHDL until one week following the final infusion. Where the patient is not treated within 7 days of an acute MI (e.g. because the patient has not had a MI or has not recently had an MI), this may be continued, e.g., for up to or at least 1, 2, 3, 4, 5, 6 months or up to or at least 1, 2, 3, 4, 5 years.

Preferably, the rHDL formulation is administered intravenously (IV) as an infusion. The IV infusion may occur over a period of about 0.5, 1, 1.5, 2, 2.5, 3, 3.5 or 4 hrs. In a particular embodiment, the IV infusion occurs over a period of about 2 hrs. In some embodiments, the amount of apolipoprotein such as Apo-AI in the rHDL formulation may be 2 g (referred to as a “low dose” or 6 g (referred to as a “high dose”). Thus preferred rates of infusion of these embodiments are about 1 g to 3 g Apo-AI per hour.

In a preferred form, the rHDL formulation is administered as a weekly 2-hour intravenous infusion for 4 consecutive weeks. The treatment period may be defined as the time from the administration of the initial dose of rHDL until one week following the final infusion. Where the patient is not treated within 7 days of an AMI (e.g. because the patient has not had a MI or has not recently had an MI), this may be continued, e.g., for up to or at least 1, 2, 3, 4, 5, 6 months or up to or at least 1, 2, 3, 4, 5 years.

A feature of the present invention is that the methods of the aforementioned aspects increase cholesterol efflux capacity (CEC) in a human patient, e.g. after an acute MI event. Cholesterol efflux capacity is an ex-vivo measure of HDL function that evaluates the ability of HDL to remove excess cholesterol from atherosclerotic plaque for transport to the liver. CEC is a correlate of MACE-independent of HDL-C, but rHDL formulations that increase or improve CEC may thereby reduce plaque burden and stabilize vulnerable plaque, which may be a more valuable effect than raising HDL alone.

Suitably, the CEC is a total cholesterol efflux capacity, preferably measured or expressed as %/4 hr. In an embodiment, the CEC is measured with an arithmetic mean of at least about 12. Preferably, the CEC comprises an ABCA1-dependent cholesterol efflux capacity (preferably measured or expressed as %/4 hr) with an arithmetic mean of at least about 5. Cholesterol efflux assays can be performed in apoB-depleted serum samples using J774 macrophages, such as as described in de le Llera-Moya et al., Arterioscler. Thromb. Vasc. Biol. 2010; 30-796-801.

Suitably, the methods disclosed herein increase total cholesterol efflux capacity by at least about 1.5-fold, up to about 2.5-fold. The increase in ABCA1-dependent cholesterol efflux capacity may be at least about 3-fold and up to about 5-fold. This greater increase in ABCA1-dependent cholesterol efflux capacity (also compared to increases in circulating Apo-AI levels), suggest that CSL112 may increase not only the amount of circulating ApoA-I but may also increase ABCA1-dependent efflux on a per ApoA-I basis. A “specific activity” of the circulating ApoA-I pool for ABCA1-dependent cholesterol efflux capacity may be calculated as the ABCA1-dependent cholesterol efflux capacity/ApoA-I ratio at the end of the infusion. By way of example, infusion of CSL 112 caused a 2.51-fold increased ratio for the 2 g dose group (0.05) and a 1.78-fold increased ratio for the 6 g dose group (0.035) compared to the placebo group (0.02). The elevation in ABCA1-dependent efflux capacity was greater than the elevation of ApoA-I. Although not wishing to be bound by theory, it is speculated that the CSL112 infusion elevates not just the quantity but also the functionality of the ApoA-I pool. The ratios of ABCA1-dependent cholesterol efflux capacity/ApoA-I were elevated with both 2 g and 6 g doses of CSL112 compared to placebo.

Suitably, increasing the CEC is not associated with, or does not cause, a substantial alteration in liver or kidney function of the human patient.

Non-limiting examples of indicators of liver function(s) include alanine aminotransferase activity (ALT), aspartate aminotransferase (AST) activity and/or bilirubin levels. Measurement of these indicators is well known in the art (see e.g. Fischbach FT, Dunning MB III, eds. (2009). Manual of Laboratory and Diagnostic Tests, 8th ed. Philadelphia: Lippincott Williams and Wilkins) and is routinely performed in medical laboratories. Kits for measuring these indicators are commercially available. Typically, liver and/or kidney function is measured after administration of the rHDL formulation. This may be compared to the liver and/or kidney function before administration of the rHDL formulation, e.g., to determine whether an alteration in function has occurred. The avoidance of a substantial alteration in liver and/or kidney function is advantageous. It is preferred to maintain the level of liver and/or kidney function that is observed prior to treatment, e.g., it is preferred that the rHDL treatment does not cause any alteration in liver and/or kidney function. In certain embodiments, the level of liver and/or kidney function may improve (i.e. give rise to indications of greater liver and/or kidney function than in the absence of treatment) but in any event it is preferred to avoid a substantial reduction in liver and/or kidney function.

In certain embodiments the methods may further comprise the step of measuring liver and/or kidney function (i) after administration of the rHDL formulation and optionally also (ii) before administration of the rHDL formulation. The kidney and/or liver function parameters before and after administration of the rHDL formulation may be compared to determine whether an alteration in liver and/or kidney function has occurred. Such methods may in certain embodiments further comprise the step of obtaining a suitable sample (e.g. blood, serum, plasma) from the human patient.

In some embodiments, a substantial alteration in liver function is an ALT of more than about 2 or 3 times the upper limit of normal (ULN); or an increase in total bilirubin of at least 1.5 to 2 times ULN. Preferably therefore the human patient does not have an ALT of more than about 2 or 3 times the upper limit of normal (ULN) either before rHDL treatment or after rHDL treatment. Further preferably the human patient does not have total bilirubin of at least 1.5 to 2 times ULN either before rHDL treatment or after rHDL treatment. In certain preferred embodiments the ALT remains substantially constant, before and after treatment (e.g. remains within 10% or 20% of the value before treatment).

Renal toxicity may be defined by serum creatinine levels. In some embodiments, a substantial alteration in kidney function is a serum creatinine greater than or equal to about 1.2-1.5 times the baseline value. Preferably therefore the human patient does not have a serum creatinine value greater than or equal to about 1.2-1.5 times the baseline value, either before rHDL treatment or after rHDL treatment. In certain preferred embodiments the serum creatinine value remains substantially constant, before and after treatment (e.g. remains within 10% or 20% of the value before treatment).

Additionally or alternatively, renal toxicity may be defined by a reduction in glomerular filtration rate (eGFR). A normal glomerular filtration rate (eGFR) of a human is at least about 90 mL/min/m² (e.g. at least about 90 mL/min/1.73 m²). This may be calculated using the CKD-EPI equation (see, e.g., Levey, 2009 Ann Intern Med May 5; 150(9): 604-612). The correlation between eGFR and kidney disease is well established and standardized in the art (see, e.g., Kidney Disease: Improving Global Outcomes (KDIGO) CKD Work Group. KDIGO 2012 Clinical Practice Guideline for the Evaluation and Management of Chronic Kidney Disease. Kidney inter., Suppl. 2013; 3: 1-150). Thus, a substantial alteration in kidney function is measured as an eGFR substantially less than 90 mL/min/m² (e.g. substantially less than 90 mL/min/1.73 m²). Mild renal impairment is typically associated with an eGFR no less than about 60 mL/min/m² (e.g. no less than about 60 mL/min/1.73 m²).

As noted above, the invention is relevant to patients with normal renal function, mild renal impairment and moderate renal impairment. Thus, it will be understood that patients having an eGFR less than 90 mL/min/1.73 m² prior to rHDL treatment (e.g., patients having mild or moderate renal impairment) may have an eGFR that is less than 90 mL/min/1.73 m² after rHDL treatment, without that eGFR level being caused by the treatment. Thus, in such cases, the rHDL treatment is not deemed to be causing “an alternation in kidney function” as used herein based solely on the eGFR being less than 90 mL/min/1.73 m². Thus, it can be useful to know the kidney function of the patient before treatment in order to determine whether the treatment has caused an alteration in kidney function.

Thus, for example, when the human patient does not have an eGFR substantially less than 90 mL/min/1.73 m² before rHDL treatment, said patient preferably does not have an eGFR substantially less than 90 mL/min/1.73 m² after rHDL treatment. Further, wherein the human patient does not have an eGFR substantially less than 60 mL/min/1.73 m² before rHDL treatment, said patient preferably does not have an eGFR substantially less than 60 mL/min/1.73 m² after rHDL treatment. Likewise, when the human patient does not have an eGFR substantially less than 30 mL/min/1.73 m² before rHDL treatment, said patient preferably does not have an eGFR substantially less than 30 mL/min/1.73 m² after rHDL treatment. Alternatively stated, in preferred embodiments, the rHDL treatment does not cause the renal status of the patient to change, according to the standard definitions as used in Kidney Disease: Improving Global Outcomes (KDIGO) CKD Work Group. KDIGO 2012 Clinical Practice Guideline for the Evaluation and Management of Chronic Kidney Disease. Kidney inter., Suppl. 2013; 3: 1-150 and referred to elsewhere herein.

Given that the kidney disease model referred to above groups patients into certain discrete categories, whilst the eGFR value is continuous, it may be useful to determine a substantial alteration in kidney function based on a change in (e.g. reduction in) eGFR after rHDL treatment of 10 or 20 or 30 mL/min/1.73 m², or more, compared to eGFR before rHDL treatment. By way of example, the patient preferably has an eGFR after treatment within 10, 20 or 30 mL/min/1.73 m² of the eGFR before rHDL treatment. For example, the patient is considered to not have a substantial alteration of kidney function wherein the eGFR after rHDL treatment is within 30, 20 or 10 mL/min/1.73 m² of the eGFR before treatment

Alternatively, renal toxicity may be defined as a requirement for renal replacement therapy.

Suitably, the rHDL formulation comprises an apolipoprotein or fragment thereof. The apolipoprotein may be any apolipoprotein which is a functional, biologically active component of naturally-occurring HDL or of a reconstituted high density lipoprotein/rHDL. Typically, the apolipoprotein is either a plasma-derived or recombinant apolipoprotein such as Apo A-I, Apo A-II, Apo A-V, pro-Apo A-I or a variant such as Apo A-I Milano. Preferably, the apolipoprotein is Apo A-I. More preferably the Apo A-I is either recombinantly derived comprising a wild type sequence or the Milano sequence or alternatively it is purified from human plasma. The apolipoprotein may be in the form of a biologically-active fragment of apolipoprotein. Such fragments may be naturally-occurring, chemically synthetized or recombinant. By way of example only, a biologically-active fragment of Apo A-I preferably has at least 50%, 60%, 70%, 80%, 90% or 95% to 100% or even greater than 100% of the lecithin-cholesterol acyltransferase (LCAT) stimulatory activity of Apo A-I.

In some general embodiments, the apolipoprotein is at a concentration from about 5 to about 50 mg/ml. This includes 5, 8, 10, 15, 20, 25, 30, 35, 40, 45 and 50 mg/ml and any ranges between these amounts. The apolipoprotein is, preferably, at a concentration from about 25 to 45 mg/ml. In particular embodiments the apolipoprotein is Apo A-I, preferably, at a concentration from about 25 to 45 mg/ml. In other embodiments, the apolipoprotein may be at a concentration of from about 5 to 20 mg/ml, e.g. about 8 to 12 mg/ml. In some embodiments the apolipoprotein is Apo A-I and its content in the rHDL formulation is from about 25 to 45 mg/mL. In other embodiments the rHDL is reconstituted following lypophilization such that the Apo A-I content in the reconstituted rHDL formulation is from about 5 to 50 mg/mL. The Apo A-I content following reconstitution of the lyophilized rHDL formulation is, preferably, at a concentration from about 25 to 45 mg/ml. In particular embodiments the Apo A-I content following reconstitution of the lyophilized rHDL formulation is about 30 to 40 mg/mL. In an embodiment the Apo A-I content following reconstitution of the lyophilized rHDL formulation is about 30 mg/mL.

Generally, the administered dosage of the rHDL formulation may be in the range of from about 1 to about 120 mg/kg body weight. Preferably, the dosage is in the range of from about 5 to about 80 mg/kg inclusive of 8 mg/kg, 10 mg/kg, 12 mg/kg, 20 mg/kg, 30 mg/kg, 40 mg/kg, 50 mg/kg, 60 mg/kg, and 70 mg/kg dosages.

In alternative embodiments, the rHDL formulation may be in the form of a “fixed dosage” formulation. Suitably, the fixed dosage apolipoprotein formulation is at a dosage that is therapeutically effective upon administration to human patients of any body weight or of any body weight in a body weight range. Accordingly, the rHDL formulation dosage is not calculated, determined or selected according to the particular body weight of the human, such as would typically occur with “weight-adjusted dosing”.

Rather, the fixed dosage apolipoprotein formulation is determined as a dosage which when administered to human patients of any body weight or of any body weight in a body weight range, would display relatively reduced inter-patient variability in terms of exposure to the apolipoprotein constituents of the apolipoprotein formulation. Relatively reduced inter-patient variability is compared to that observed or associated with weight-adjusted dosing of a patient population.

Variability of exposure may be expressed or measured in terms of the variation in exposure of patients to apolipoprotein following administration of the fixed dosage apolipoprotein formulation. Preferably, the variability is that which would occur when the fixed dosage apolipoprotein formulation is administered to human patients over a weight range compared to the variability that would occur for weight-adjusted dosages administered to human patients over the same weight range as the fixed dosage patients. In some embodiments, exposure to apolipoprotein may be measured as average exposure (e.g. mean or median exposure), total exposure (e.g. amount integrated over time of exposure) or maximum exposure level (e.g. Cmax). Generally, the weight or weight range is 20, 30, 40, 50, 60, 70, 80, 90, 100, 110, 120, 130, 140, 150, 160, 170, 180, 190 or 200 kg, or any range between these values. Preferably, the weight or weight range is 20-200 kg, 20-60 kg, 40-160 kg, 50-80 kg, 60-140 kg, 70-80 kg, 80-120 kg, 100-180 kg or 120-200 kg.

Suitably, the variability is less than 100% or preferably 99%, 98%, 97%, 96% 95%, 94%, 93%, 92%, 91%, or less than 90%, 85% or 80% of the variability that occurs with weight-adjusted dosing. Variability may be calculated and expressed by any statistical representation known in the art, including as a co-efficient of variation (e.g. % CV), standard deviation, standard error or the like, although without limitation thereto.

Notwithstanding administration of a fixed dosage apolipoprotein formulation to patients of markedly different body weights, the exposure of the patients to apolipoprotein is surprisingly uniform. Accordingly it is proposed that the therapeutic efficacy of the fixed dosage apolipoprotein formulation will not be substantially compromised or reduced compared to a weight-adjusted dosage.

By way of example only, it has been shown that there is no difference in total exposure to apolipoprotein upon administration of a fixed dosage apolipoprotein formulation to patients in the 60-120 kg weight range. Furthermore, Cₘₐₓ for apolipoprotein decreased by an average of 16% over the 60-120 kg weight range.

In comparison, for weight-adjusted dosing regimes using the same apolipoprotein formulation, a doubling of body weight from 60 kg to 120 kg requires a doubling of the dosage of apolipoprotein and increased ApoA-I exposure.

Fixed dosage apolipoprotein formulations may be administered in multiple doses at any suitable frequency including daily, twice weekly, weekly, fortnightly or monthly. Fixed dosage apolipoprotein formulations may be administered by any route of administration known in the art, such as intravenous administration (e.g., as a bolus or by continuous infusion over a period of time such as over 60, 90, 120 or 180 minutes), by intra-muscular, intra-peritoneal, intra-arterial including directly into coronary arteries, intra-cerebrospinal, sub-cutaneous, intra-articular, intra-synovial, intra-thecal, oral, topical, or inhalation routes. Typically, fixed dosage apolipoprotein formulations are administered parenterally, such as by intravenous infusion or injection.

Preferred fixed dosages include 0.1-15 g, 0.5-12 g, 1-10 g, 2-9 g, 3-8 g, 4-7 g or 5-6 g of apolipoprotein. Particularly preferred fixed dosages include 1-2 g, 3-4 g, 5-6 g or 6-7 g of apolipoprotein. Non-limiting examples of specific fixed dosages include 0.25 g, 0.5 g, 1 g, 1.7 g, 2 g, 3.4 g, 4 g, 5.1 g, 6 g, 6.8 g and 8 g of apolipoprotein. Accordingly, a vial of fixed dosage rHDL formulation preferably comprises a lyophilized rHDL formulation with an apolipoprotein content of 0.25 g, 0.5 g, 1, 2, 2.5, 3, 3.5, 4, 4.5, 5, 5.5, 6, 6.5, 7, 8 or 10 g per vial. More preferably the apolipoprotein content is either 2, 4, 6, 8, or 10 g per vial. A particularly preferred vial comprises 6 g or more of rHDL formulation.

A non-limiting example of fixed dosage CSL112 rHDL formulations may be found in International Publication WO2013/090978.

The lipid in the rHDL formulation may be any lipid which is a functional, biologically active component of naturally occurring HDL or of reconstituted high density lipoprotein (rHDL). Such lipids include phospholipids, cholesterol, cholesterol-esters, fatty acids and/or triglycerides. Preferably, the lipid is at least one charged or non-charged phospholipid or a mixture thereof.

In a preferred embodiment the rHDL formulation according to the present invention comprises a combination of a detergent and a non-charged phospholipid. In an alternative preferred embodiment the rHDL formulation comprises a charged phospholipid but no detergent at all. In a further preferred embodiment the rHDL formulation comprises charged and non-charged lipids as well as a detergent.

As used herein, “non-charged phospholipids”, also called neutral phospholipids, are phospholipids that have a net charge of about zero at physiological pH. Non-charged phospholipids may be zwitterions, although other types of net neutral phospholipids are known and may be used. “Charged phospholipids” are phospholipids that have a net charge at physiological pH. The charged phospholipid may comprise a single type of charged phospholipid, or a mixture of two or more different, typically like-charged phospholipids. In some examples, the charged phospholipids are negatively charged glycophospholipids.

The rHDL formulation may also comprise a mixture of different lipids, such as a mixture of several non-charged lipids or of a non-charged lipid and a charged lipid. Examples of phospholipids include phosphatidylcholine (lecithin), phosphatidic acid, phosphatidylethanolamine (cephalin), phosphatidylglycerol (PG), phosphatidylserine (PS), phosphatidylinositol (PI) and sphinogomyelin (SM) or natural or synthetic derivatives thereof. Natural derivatives include egg phosphatidylcholine, egg phosphatidylglycerol, soy bean phosphatidylcholine, hydrogenated soy bean phosphatidylcholine, soy bean phosphatidylglycerol, brain phosphatidylserine, sphingolipids, brain sphingomyelin, egg sphingomyelin, galactocerebroside, gangliosides, cerebrosides, cephalin, cardiolipin and dicetylphospate. Synthetic derivatives include dipalmitoylphosphatidylcholine (DPPC), didecanoylphosphatidyl-choline (DDPC), dierucoylphosphatidylcholine (DEPC), dimyristoylphosphatidylcholine (DLPC), palmitoyloleoylphosphatidylcholine (PMPC), palmitoylstearoyl-phosphatidylcholine (PSPC), dioleoylphosphatidylethanolamine (DOPE), dilauroylphosphatidylglycerol (DLPG), distearoylphosphatidylglycerol (DSPG), dioleoylphosphatidylglycerol (DOPG), palmitoyloleoylphosphatidylglycerol (POPG), dimyrstolyphosphatidic acid (DMPA), dipalmitoylphosphatidic acid (DPPA), distearoyl-phosphatidic acid (DSPA), dipalmitoylphosphatidylserine (DPPS), distearoylphosphatidylethanolamine (DSPE), dioleoylphosphatidylethanolamine (DOPE), dioleoylphosphatidylserine (DOPS), dipalmitoylsphingomyelin (DPSM) and distearoylsphingomyelin (DSSM).

The phospholipid can also be a derivative or analogue of any of the above phospholipids. Best results could be obtained with phosphatidylcholine. In another embodiment the lipids in the formulation according to the present invention are sphingomyelin and a negatively charged phospholipid, such as phosphatidylglycerol (e.g. DPPG).

The rHDL formulation may comprise a mixture of sphingomyelin and phosphatidylglycerol (particularly DPPG). In these embodiments, the sphingomyelin and the phosphatidylglycerol may be present in any suitable ratio, e.g. from 90:10 to 99:1 (w:w), typically 95:5 to 98:2 and most typically 97:3. In other embodiments the rHDL formulation does not comprise a mixture of sphingomyelin and phosphatidylglycerol (particularly DPPG).

Suitably, the molar ratio of apolipoprotein:lipid is typically from about 1:20 to about 1:120, and preferably from about 1:20 to about 1:100, more preferably from about 1:20 to about 1:75 (mol:mol), and in particular from 1:45 to 1:65. This range includes molar ratios such as about 1:25, 1:30, 1:35, 1:40, 1:45, 1:50, 1:55, 1:60, 1:65, 1:70, 1:75, 1:80, 1:85, 1:90, 1:95 and 1:100. A particularly advantageous ratio of apolipoprotein:lipid is from 1:40 to 1:65 (mol:mol). This ensures that the rHDL formulation according to the present invention comprises a lipid at a level which does not cause liver toxicity.

In other embodiments, the molar ratio of apolipoprotein:lipid may be in a range from about 1:80 to about 1:120. For example, the ratio may be from 1:100 to 1:115, or from 1:105 to 1:110. In these embodiments, the molar ratio may be for example from 1:80 to 1:90, from 1:90 to 1:100, or from 1:100 to 1:110. In alternate embodiments the molar ratio of apolipoprotein:lipid is not in a range from about 1:80 to about 1:120.

Suitably, the rHDL formulation comprises a stabilizer. Typically, the stabilizer is present in a concentration from about 1.0% to about 6.0% e.g. from 1.0, 1.1, 1.2 or 1.3% to 5.5, 5.6, 5.7, 5.8, 5.9, or 6.0%, preferably from about 1.0% to less than 6.0%, e.g. from about 1.0% to 5.9% (w/w of rHDL formulation). Preferably from about 3.0% to less than 6.0%, e.g. from about 3.0% to 5.9%, preferably from about 4.0 to 5.9%, preferably, from about 4.0% to 5.5%, preferably 4.3 to 5.3%, preferably 4.3 to 5.0%, and most preferably from 4.6 to 4.8% (w/w) and in said formulation the ratio between the apolipoprotein and the lipid is preferably from about 1:20 to about 1:75, more preferably from about 1:45 to about 1:65 (mol:mol). The lyophilization stabilizer is preferably a sugar (e.g. a disaccharide such as sucrose).

This relatively low amount of stabilizer may reduce the risk of renal toxicity. It is also particularly suitable for patients receiving contrast agents during acute coronary syndrome therapy (ACS), since these agents may compete with stabilizer for clearance in the kidneys.

Preferably, the stabilizer is a “lyophilization stabilizer”, which is a substance that stabilizes protein during lyophilization. A preferred lyophilization stabilizer comprises a sugar. For example, disaccharides such as sucrose are particularly suitable sugars for use as the lyophilization stabilizer. Other disaccharides that may be used include fructose, trehalose, maltose and lactose. In addition to disaccharides, trisaccharides like raffinose and maltotriose may be used. Larger oligosaccharides may also be suitable, e.g. maltopentaose, maltohexaose and maltoheptaose. Alternatively, monosaccharides like glucose, mannose and galactose may be used. These mono-, di-, tri- and larger oligo-saccharides may be used either alone or in combination with each other.

In some other embodiments the lyophilization stabilizer is a sugar alcohol, an amino acid, or a mixture of sugar and sugar alcohol and/or amino acid.

A particular sugar alcohol is mannitol. Other sugar alcohols that may be used include inositol, xylitol, galactitol, and sorbitol. Polyols like glycerol may also be suitable.

A mixture of sucrose and mannitol may be used. The sugar and the sugar alcohol may be mixed in any suitable ratio, e.g. from about 1:1 (w:w) to about 3:1 (w:w), and in particular about 2:1 (w:w). Ratios less than 2:1 are particularly envisaged, e.g. less than 3:2. Typically, the ratio is greater than 1:5, e.g. greater than 1:2 (w:w). In some embodiments the formulation comprises less than 4% sucrose and 2% mannitol (w/w of rHDL formulation), for example 3% sucrose and 2% mannitol. In some embodiments the formulation comprises 4% sucrose and less than 2% mannitol. In some embodiments the formulation comprises less than 4% sucrose and less than 2% mannitol e.g. about 1.0% to 3.9% sucrose and about 1.0% to 1.9% (w/w) mannitol.

Amino acids that may be used as lyophilization stabilizers include proline, glycine, serine, alanine, and lysine. Modified amino acids may also be used, for example 4-hydroxyproline, L-serine, sodium glutamate, sarcosine, and γ-aminobutyric acid. Proline is a particularly suitable amino acid for use as a lyophilization stabilizer. In some embodiments, the lyophilization stabilizer comprises a mixture of a sugar and an amino acid. For example, a mixture of sucrose and proline may be used. The sugar and the amino acid may be mixed in any suitable ratio, e.g. from about 1:1 to about 3:1 (w:w), and in particular about 2:1 (w:w). Ratios less than 2:1 are particularly envisaged, e.g. less than 3:2 (w:w). Typically, the ratio is greater than 1:5, e.g. greater than 1:2 (w:w). Preferably the amino acid is present in a concentration of from about 1.0 to about 2.5% e.g. from 1.0, 1.2, or 1.3 to 2.0, 2.1, 2.2, 2.3, 2.4, or 2.5% (w/w of rHDL formulation). In some embodiments the formulation comprises 1.0% sucrose and 2.2% proline, or 3.0% sucrose and 1.5% proline, or 4% sucrose and 1.2% proline. The amino acid may be added to the sugar to maintain an isotonic solution. Solutions with an osmolality of greater than 350 mosmol/kg are typically hypertonic, while those of less than 250 mosmol/kg are typically hypotonic. Solutions with an osmolality of from 250 mosmol/kg to 350 mosmol/kg are typically isotonic.

The ratio between the apolipoprotein and the lyophilization stabilizer is usually adjusted so that the ratio is from about 1:1 to about 1:7 (w:w). More preferably, the ratio is from about 1:1 to about 1:3, in particular about 1:1.1 to about 1:2. In specific embodiments the rHDL formulations thus have ratios of 1:1.1, 1:1.2, 1:1.3, 1:1.4, 1:1.5, 1:1.6, 1:1.7, 1:1.8, 1:1.9 or 1:2 (w:w). It is however contemplated that for particular embodiments where there are low amounts of protein (e.g. <20 mg/mL) that the ratio between the apolipoprotein and the lyophilization stabilizer can be extended to as much as about 1:7 (w:w), e.g. about 1:4.5 (w:w).

Reference is made to International Publication WO2014/066943 which provides non-limiting, particular examples and discussion of lyophilization stabilizers in the context of the CSL112 rHDL formulation.

In some optional embodiments, the rHDL formulation comprises a detergent. The detergent may be any ionic (e.g. cationic, anionic, zwitterionic) detergent or non-ionic detergent, inclusive of bile acids and salts thereof, suitable for use in rHDL formulations. Ionic detergents may include bile acids and salts thereof, polysorbates (e.g. PS80), 3-[(3-Cholamidopropyl)dimethylammonio]-1-propane-sulfonate (CHAPS), 3-[(3-Cholamidopropyl)dimethylammonio]-2-hydroxy-1-propanesulfonate (CHAPSO), cetyl trimethyl-ammonium bromide, lauroylsarcosine, tert-octyl phenyl propanesulfonic acid and 4′-amino-7-benzamido-taurocholic acid.

Bile acids are typically dihydroxylated or trihydroxylated steroids with 24 carbons, including cholic acid, deoxycholic acid, chenodeoxycholic acid or ursodeoxycholic acid. Preferably, the detergent is a bile salt such as a cholate, deoxycholate, chenodeoxycholate or ursodeoxycholate salt. A particularly preferred detergent is sodium cholate. The concentration of the detergent, in particular of sodium cholate, is preferably 0.3 to 1.5 mg/mL. In some embodiments of the invention the rHDL formulation comprises cholate levels of about 0.015-0.030 g/g apolipoprotein. The bile acid concentration can be determined using various methods including colorimetric assay (for example, see Lerch et. al., 1996, Vox Sang. 71:155-164; Sharma, 2012, Int. J. Pharm Biomed. 3(2), 28-34; & Gallsauren test kit and Gallsauren-Stoppreagens (Trinity Biotech)). In some embodiments of the invention the rHDL formulation comprises cholate levels of 0.5 to 1.5 mg/mL as determined by colorimetric assay.

In a preferred embodiment, the rHDL formulation disclosed herein has a pH in the range of 6 to 8, preferably within the range of 7 to 8. Even more preferably the pH is in the range of 7.3 to 7.7.

In a preferred embodiment, the rHDL formulation is lyophilized. Due to the presence of the hereinbefore described lyophilization stabilizer, preferably sucrose, in combination with the apolipoprotein:lipid ratio, the lyophilisation produces a stable powder having a long shelf life. This powder may be stored, used directly or after storage as a powder or used after rehydration to form the reconstituted high density lipoprotein formulation.

The invention may be used with rHDL manufactured at large scale production using human plasma derived ApoA-I. The lyophilized product may be prepared for bulk preparations, or alternatively, the mixed protein/lipid solution may be apportioned in smaller containers (for example, single dose units) prior to lyophilization, and such smaller units may be used as sterile unit dosage forms. The lyophilized formulation can be reconstituted in order to obtain a solution or suspension of the protein-lipid complex, that is the reconstituted high density lipoprotein. The lyophilized powder is rehydrated with an aqueous solution to a suitable volume. Preferred aqueous solutions are water for injection (WFI), phosphate-buffer saline or a physiological saline solution. The mixture can be agitated to facilitate rehydration. Preferably, the reconstitution step is conducted at room temperature.

It is well known to the person skilled in the art how to obtain a solution comprising the lipid, and the apolipoprotein, such as described in WO 2012/000048.

The lyophilized rHDL formulation of the present invention may be formed using any method of lyophilization known in the art, including, but not limited to, freeze drying, i.e. the apolipoprotein/lipid-containing solution is subjected to freezing followed by reduced pressure evaporation.

The lyophilized rHDL formulations that are provided can retain substantially their original stability characteristics for at least 2, 4, 6, 8, 10, 12, 18, 24, 36 or more months. For example, lyophilized rHDL formulations stored at 2-8° C. or 25° C. can typically retain substantially the same molecular size distribution as measured by HIPLC-SEC when stored for 6 months or longer. Particular embodiments of the rHDL formulation can be stable and suitable for commercial pharmaceutical use for at least 6 months, 12 months, 18 months, 24 months, 36 months or even longer when stored at 2-8° C. and/or room temperature.

It will also be appreciated that the method and/or the rHDL formulation disclosed herein may include one or more additional therapeutic agents. Likewise the reconstituted high density lipoprotein (rHDL) formulation as disclosed herein for use in the specific methods as disclosed herein may be used with one or more additional therapeutic agents. Suitably, the one or more additional therapeutic agents may assist or facilitate treatment, prevention or reduction in risk of an acute myocardial infarction (MI) event and/or MACE and/or increasing cholesterol efflux capacity (CEC) in a human patient, although without limitation thereto.

The one or more additional therapeutic agents may include: one or more lipid-modifying agents; one or more cholesterol absorption inhibitors; one or more anti-coagulants; one or more anti-hypertensive agents; and one or more bile acid binding molecules.

Lipid-modifying agents may decrease or reduce LDL and/or triglycerides and/or increase HDL. Non-limiting examples include HMG-CoA reductase inhibitors, fibrates (e.g. fenofibrate, gemfibrozil), proprotein convertase subtilisin/kexin type 9 (PCSK9) inhibitors and niacin.

Non-limiting examples of HMG-CoA reductase inhibitors include “statins” such as lovastatin, rosuvastatin, atorvastatin, pitavastatin and simvastatin, although without limitation thereto.

A non-limiting example of a cholesterol absorption inhibitor includes ezetimibe, which may be administered alone or together with a statin, such as hereinbefore described.

Non-limiting examples of anti-coagulants include warfarin, vitamin K antagonists, heparin or derivatives thereof, factor Xa inhibitors and thrombin inhibitors, although without limitation thereto.

Non-limiting examples of anti-hypertensive agents include angiotensin converting enzyme (ACE) inhibitors (e.g enalapril, raimipril, captopril etc), angiotensin II receptor antagonists (e.g irbesartan), renin inhibitors, adrenergic receptor antagonists, calcium channel blockers, vasodilators, benzodiazepines and diuretics (e.g thiazides), although without limitation thereto.

Non-limiting examples of bile acid binding molecules or “sequestrants” include cholestyramine, colestipol and colesevelam, although without limitation thereto.

Suitable dosages of the one or more additional therapeutic agents may readily be determined by reference to existing, established safe dosage regimes for these agents, which may readily be altered or modified by practitioners in the art.

It will be understood that the one or more additional therapeutic agents may be incorporated into the rHDL formulation disclosed herein or may be administered separately according to the method of treatment or therapeutic use disclosed herein. This may include administration before or after administration of the rHDL formulation disclosed herein, at least within 24, 18, 12, 6, 3, 2 or 1 hours of administration of the rHDL formulation.

So that particular embodiments of the invention may be readily understood and put into practical effect, reference is made to the following non-limiting Examples.

### EXAMPLES

### Abbreviations

ACS: Acute Coronary Syndrome
AE: Adverse Event
AKI Acute Kidney Injury
AMI: Acute Myocardial Infarction
ApoA-I: Apolipoprotein A-I
AST: Aspartate Aminotransferase
AUC: Area Under the Curve
BARC: Bleeding Academic Research Consortium
CAD: Coronary Artery Disease
CEC: Cholesterol Efflux Capacity
CKD-EPI: Chronic Kidney Disease Epidemiology Collaboration
CL: Systemic Clearance
Cₘₐₓ: Maximum Concentration in Plasma
CV: Cardiovascular
DSMB: Data Safety Monitoring Board
eGFR: Estimated Glomerular Filtration Rate
HAV: Hepatitis A Virus
HBV: Hepatitis B Virus
HCV: Hepatitis C Virus
HDL: High Density Lipoprotein
HIV: Human Immunodeficiency Virus
ITT: Intention-to-Treat
LVEF: Left Ventricular Ejection Fraction
MACE: Major Adverse Cardiovascular Events
MI: Myocardial Infarction
Mod RI: Moderate renal impairment
NAT: Nucleic Acid Testing
NRF: Normal renal function
NYHA: New York Heart Association
PC: Phosphatidylcholine
PCI: Percutaneous Coronary Intervention
PK/PD: Pharmacokinetics/Pharmacodynamics
RI: renal impairment
SAE: Serious Adverse Event
t_{1/2}: Half-life
TEAE: Treatment Emergent Adverse Event
Tₘₐₓ: Time to Reach Maximum Concentration in Plasma
ULN: Upper Limit of Normal
Vₛₛ: Volume of Distribution at Steady State

### Example 1

CSL112 is a plasma-derived ApoA-I, the primary functional component of HDL, reconstituted into disc-shaped lipoproteins with phosphatidylcholine and stabilized with sucrose²⁴. Initial studies of CSL112 have demonstrated a significant dose-dependent increase in plasma ApoA-I, and a dose-dependent increase in total and ABCA1-dependent cholesterol efflux capacity²⁵⁻²⁷. A favorable safety profile has been demonstrated in the clinical program to date, including patients with stable atherosclerotic disease, although it has not been characterized in patients with acute MI²⁷. A prototype formulation of CSL112 was discontinued from development due to the occurrence of transient elevations of hepatic enzymes presumed related to the phosphatidylcholine excipient content^{28, 29}. Risk of renal toxicity has been described with high doses of intravenous sucrose. We therefore assessed both hepatic and renal function following infusion of this lower phosphatidylcholine and low-sucrose-containing preparation of CSL112 in MI patients.

The Apo-I Event reductinG in Ischemic Syndromes I (AEGIS-I) trial was a multi-center, randomized, placebo-controlled, dose-ranging phase 2b clinical trial, with the primary objective to assess safety and tolerability, and secondary and exploratory objectives including time-to-first occurrence of MACE, as well as the pharmacokinetics and pharmacodynamics of 4 weekly administrations of two doses of CSL112 compared with placebo among patients with acute MI and either normal renal function or mild renal impairment (ClinicalTrials.gov: NCT02108262).

### Methods

### Study Oversight

AEGIS-I was a randomized, double-blind, placebo-controlled, dose-ranging, phase 2b trial designed in collaboration between the study sponsor (CSL Behring) and members of the executive and steering committee. Statistical analyses were conducted independently by the PERFUSE Study Group using the SD™ datasets. The executive committee drafted all versions of the manuscript and agreed to the content of the final version. The sponsor had the opportunity to review and comment on the final draft of the manuscript, but had no editorial authority. The study design was in accordance with the 1964 Declaration of Helsinki and its later amendments, and approved by the appropriate national and institutional regulatory agencies and ethics committees. An independent data and safety monitoring board (DSMB) monitored the trial and reviewed unblinded data.

### Study Population

Men and women, at least 18 years of age, with a clinical presentation consistent with a type I (spontaneous) MI within the past 7 days, and who had either normal renal function or mild renal impairment, were enrolled. The criteria for MI were based on the third universal definition of MI³⁰. Normal renal function was defined as an eGFR ≥90 mL/minute/1.73 m², and mild renal impairment was defined as eGFR <90 mL/minute/1.73 m² and ≥60 mL/minute/1.73 m².

Major exclusion criteria included evidence of current hepatobiliary disease, baseline moderate or severe chronic kidney disease, history of contrast-induced acute kidney injury, or ongoing hemodynamic instability. Among subjects who underwent angiography and were administered a contrast agent, stable renal function at least 12 hours following contrast administration (i.e. no increase in serum creatinine ≥0.3 mg/dL from the pre-contrast value) was required for enrollment. The study was approved by an institutional review committee and all subjects provided written informed consent prior to enrollment.

### Study Protocol

The Food and Drug Administration mandated a review of renal and hepatic safety by the DSMB after the first 9 patients were enrolled, and following DSMB approval, enrollment in the main study was initiated. Eligible patients were first stratified by renal function (either normal renal function or mild renal impairment), and were then randomly assigned with a 1:1:1 ratio to one of three treatment groups: either low dose CSL112 (2 g ApoA-I/dose), high dose CSL112 (6 g ApoA-I/dose), or placebo. The study drug was administered as a weekly 2-hour intravenous infusion for 4 consecutive weeks (on study days 1, 8, 15, and 22). The active treatment period was defined as the time from the administration of the first dose of study drug (study day 1) until one week following the last infusion (study day 29).

Patients were routinely evaluated at pre-determined intervals from screening until the final follow-up visit. Evaluations included physical examinations, serum creatinine, total bilirubin, alkaline phosphatase, ALT, AST, BUN, Cr, glucose, metabolic, cardiovascular, and lipid biomarkers, markers of immunogenicity, and assessments of infusion site, bleeding, and adverse events. The occurrence of major adverse cardiovascular events (MACE) was also monitored for all subjects for up to one year after randomization or until the last randomized subject completed the study day 112 visit.

Plasma concentrations of apoA-I, and ex-vivo cholesterol efflux were measured at several time points. In addition, a pharmacokinetics/pharmacodynamics (PK/PD) substudy was conducted among 63 patients. Subjects included in the substudy were equally stratified by renal function and were randomly assigned with a ratio of 2:3:3 to either placebo, low dose CSL112 (2 g apoA-I/dose), or high dose CSL112 (6 g apoA-I/dose), respectively. The ability of plasma to mediate cholesterol efflux from cultured J774 cells was measured as previously described²⁶. These assays measure both total cholesterol efflux capacity as well as the efflux that may be attributed to the ABCA1 transporter. Both efflux measures are presented as percent of cellular cholesterol content. Additional details of the AEGIS-I trial design have been previously published³¹.

### Co-Primary Safety Endpoints

The co-primary safety endpoints were rates of hepatotoxicity and renal toxicity. Hepatotoxicity was defined as the incidence of either ALT >3× the upper limit of normal (ULN) or total bilirubin >2×ULN that was confirmed on repeat measurement. Renal toxicity was defined as either a serum creatinine ≥1.5× the baseline value that was confirmed upon repeat measurement or a new-onset requirement for renal replacement therapy. Both hepatic and renal safety endpoints were evaluated from baseline (prior to the first infusion) through the end of the active treatment period (study day 29). All measures for the co-primary safety endpoints were based on central laboratory values.

### Secondary and Exploratory Endpoints

Secondary and exploratory efficacy endpoints were assessed in the Intent to Treat (ITT) population (all patients randomized including those who did not receive study drug) and included the time-to-first occurrence of MACE, which was defined as the composite of cardiovascular death, nonfatal MI, ischemic stroke, or hospitalization for unstable angina, from randomization until the last treated subject completed study day 112. All MACE were adjudicated by an independent clinical events committee that was blinded to treatment assignment.

Bleeding was assessed as a secondary safety endpoint as the majority of subjects were anticipated to be treated with dual anti-platelet therapy post-MI. Measured and baseline-corrected plasma apoA-I concentrations, analyses of pharmacodynamic characteristics of CSL112 including changes in total and ABCA1-dependent cholesterol efflux measures (ex-vivo), as well as lipid, metabolic, and cardiovascular biomarkers were assessed. Additional pre-specified endpoints have been previously described³¹

### Statistical Analysis

Statistical analyses were conducted using SAS© version 9.4. All safety endpoints were evaluated in the safety population, which consisted of randomized subjects who received at least one partial dose of the study drug. In the safety population, subjects were classified according to the actual treatment they received and their true renal stratum. Efficacy endpoints were evaluated in the ITT population, which consisted of all randomized subjects. In the ITT population, subjects were classified according to the treatment they were randomized to and according to the renal function stratum they were randomized from, regardless of actual treatment or true renal function stratum. Additional populations, such as the PK analysis population, PK/PD analysis population, and biomarker analysis population, were pre-defined in the study protocol.

The Newcombe-Wilson score method was used to calculate the two-sided 95% confidence intervals of the difference in rates (CSL 112 minus placebo) for the co-primary safety endpoints. The upper bound of the two-sided 95% confidence interval was specified for testing the co-primary endpoints, comparing with the specified thresholds for hepatic and renal endpoints for the non-inferiority assessment. This gives a one-sided 2.5% Type I error for each of the hepatic and renal endpoints and was based on an application of the Bonferroni method to control the overall Type I error at 5%. Non-inferiority criteria were pre-specified to be met for the rate difference if the upper bound of the 95% confidence interval was ≤4% in hepatic outcomes and ≤5% in renal outcomes for a pairwise treatment group comparison. Bleeding rates were compared among the three groups.

Although not powered to detect differences in MACE, secondary and exploratory MACE outcomes were evaluated by calculating differences in time-to-first MACE between the treatment groups using a Cox proportional hazards model, with treatment assignment and baseline renal function stratum as covariates. A two-sided log rank test p-value was calculated for each CSL112 dose vs. placebo with stratification by renal function. No formal hypothesis testing for MACE was intended.

### Results

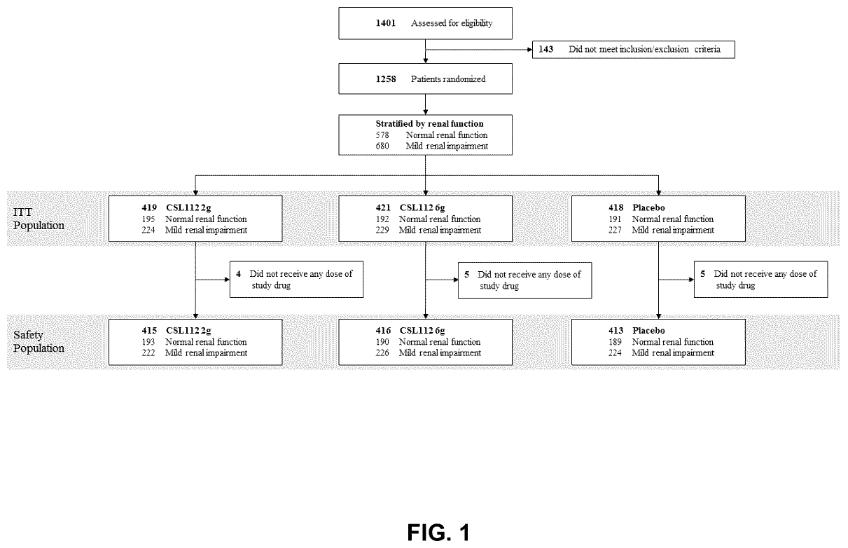

From January 2015 through November 2015, a total of 1,258 patients in 16 countries were randomized, of whom 1244 (99.6%) received at least one dose of study drug and 1147 (91.2%) received all 4 infusions. A total of 680 (54.1%) patients were stratified to the normal renal function stratum, and 578 (45.9%) were stratified to the mild renal impairment stratum (FIG. 1). For the index event 61.6% of patients experienced STEMI and 38.4% experienced NSTEMI. The median duration from the index event to randomization was 4 days, and while 24 to 34 patients per treatment group had one year of follow-up, the median duration of follow-up was 7.5 (IQR 5.8, 9.7) months. Baseline characteristics were well-balanced between the 3 treatment groups (Table 1).

### Co-Primary Endpoints Results

During the active treatment period, the co-primary safety endpoint of hepatic impairment occurred in 0 (0.0%) patients in the placebo group, 4/415 (1.0%) of patients in the 2 g dose group (p=0.12 vs placebo), 2/416 (0.5%) of patients in the 6 g dose group (p=0.50 vs placebo). Both dose comparisons to placebo were not significantly different and were within the pre-specified margin of ≤4% (Table 2). There were no Hy's law cases (i.e. concomitant elevation of ALT/AST and bilirubin with no other reason to explain the combination) in the trial. Results from two pre-specified sensitivity analyses, including patients with elevated baseline bilirubin and all elevated values regardless of confirmation values, were consistent with the results of primary safety analysis (Table 7).

The co-primary safety endpoint of renal impairment occurred in 1/413 (0.2%) patient in the placebo group, 0/415 (0.0%) of patients in the 2 g dose group (p=0.50 vs placebo), and 3/416 (0.7%) of patients in the 6 g dose group (p=0.62 vs placebo). Both dose comparisons to placebo were not significantly different and were within the pre-specified margin of ≤5% (Table 2). Additional pre-specified exploratory safety analyses and post-hoc analyses are shown in Tables 8 and 9.

### Secondary and Exploratory Endpoints Results

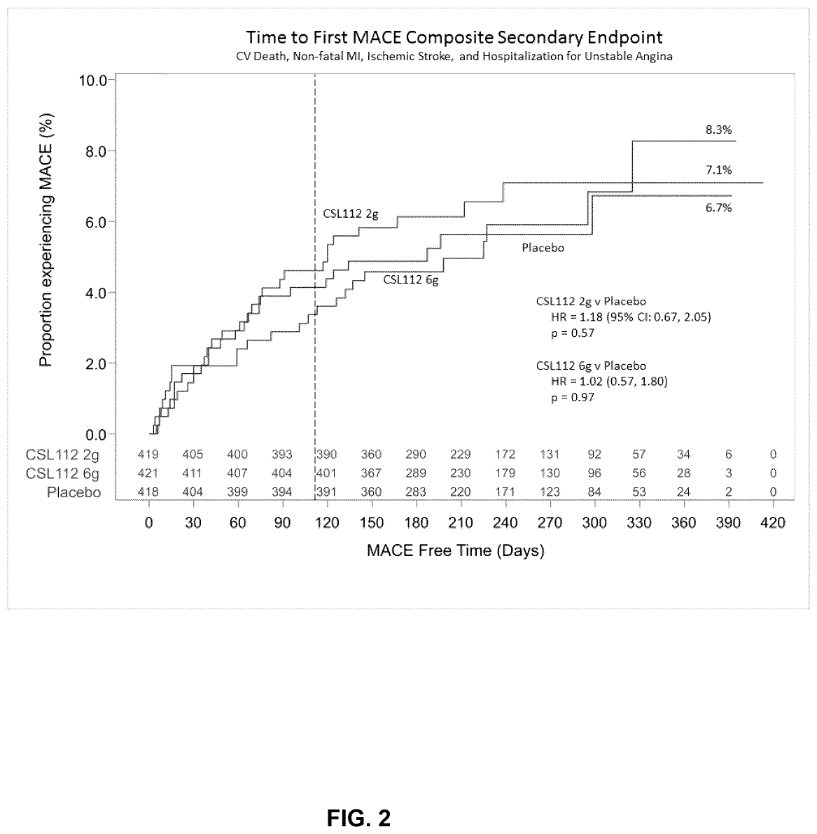

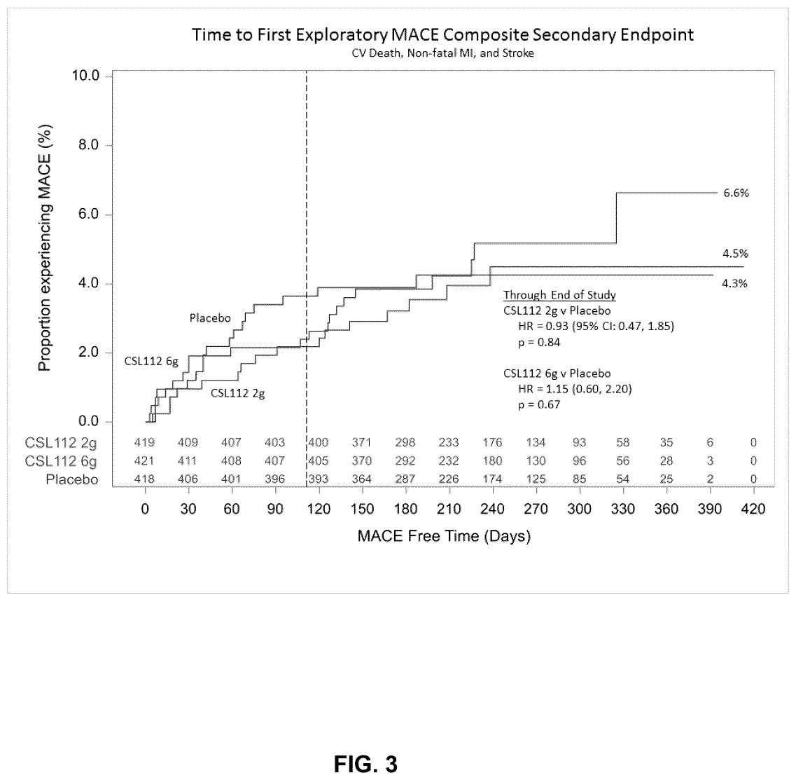

Through 12 months of follow-up, the risk of the MACE Composite Secondary Endpoint (CV Death, non-fatal MI, ischemic stroke and hospitalization for unstable angina) with CSL112 therapy as compared with placebo was similar (low dose [2 g](27/419, 6.4%) vs. placebo (23/418, 5.5%): hazard ratio, 1.18; 95% CI, 0.67 to 2.05; p=0.72) and high dose [6 g]: (24/421, 5.7%, hazard ratio, 1.02; 95% CI, 0.57 to 1.80; p=0.52) (FIG. 2). Similar risks among treatment groups for the exploratory MACE composite endpoints were observed including in the traditional phase 3 endpoint of cardiovascular death, nonfatal MI and stroke (FIG. 3). As for the secondary MACE composite endpoint, the majority of additional exploratory MACE endpoints were similar among treatment groups. There was a difference in the number of cardiovascular related deaths when comparing CSL112 6 g apoA-I (n=4, 1.0%; p=0.0477) vs. placebo (n=0, 0.0%), but this was not seen when comparing CSL112 2 g apoA-I (n=2, 0.5%; p=0.32) to placebo. However, the number of patients experiencing cardiovascular related deaths was low (Table 3). Similarly, a difference in the number of heart failure events was observed when comparing CSL112 6 g apoA-I (n=4, 1.0%; p=0.2525) to placebo (n=1, 0.2%) and CSL112 2 g apoA-I (n=5, 1.2%; p=0.1205) to placebo. The number of patients experiencing heart failure was low (Table 3).

The rates of all grades of BARC bleeding were low and were comparable between the 3 arms (Table 4). Drug hypersensitivity reactions and infusion site reactions were well balanced across groups. Overall, the rates of serious and life-threatening adverse events and serious adverse events leading to drug discontinuation were relatively low and comparable across all groups (Tables 10 and 11).

Baseline plasma concentrations of apoA-I, cholesterol efflux capacity as well as lipid and cardiovascular biomarkers were similar among the three treatment groups (Table 5). Infusion of CSL112 caused a dose-dependent elevation of both apoA-I and cholesterol efflux capacity (Table 6). The 2 g dose elevated apoA-I 1.29-fold and total cholesterol efflux capacity 1.87-fold while the 6 g dose elevated apoA-I 2.06-fold and total cholesterol efflux capacity 2.45-fold. Consistent with prior findings, the elevation of ABCA1-dependent cholesterol efflux capacity (3.67-fold for the 2 g dose, 4.30-fold for the 6 g dose) was substantially greater than either the elevation of apoA-I or total cholesterol efflux capacity suggesting that CSL112 may increase not only the amount of circulating apoA-I but may also increase the activity for ABCA1-dependent efflux on a per apoA-I basis²⁶. We assessed this “specific activity” of the circulating apoA-I pool for ABCA1-dependent cholesterol efflux capacity by calculating the ABCA1-dependent cholesterol efflux capacity/apoA-I ratio at the end of the infusion. Infusion of CSL112 caused a 2.51-fold increased ratio for the 2 g dose group (0.05) and a 1.78-fold increased ratio for the 6 g dose group (0.035) compared to the placebo group (0.02)²⁶. The elevation in ABCA1-dependent efflux capacity was greater than the elevation of apoA-I. Although this ratio is not a validated measure, it could be speculated that the infusion elevates not just the quantity but also the functionality of the apoA-I pool. Indeed, the ratios of ABCA1-dependent cholesterol efflux capacity/apoA-I were elevated with both doses of CSL112 compared to placebo (Table 9).

### Discussion

Infusions of CSL112, a reconstituted plasma-derived apoA-I, at both low [2 g] and high [6 g] doses, administered as 4 weekly infusions beginning within 7 days of acute MI, were not associated with alterations in either liver or kidney function. This was the first study in which CSL112 was administered to acute MI patients, and the first time it was added to acute MI standard of care. Establishing safety and feasibility in the acute MI setting was important prior to initiation of a large-scale phase 3 outcomes trial. The results from AEGIS-I suggest that the current formulation of CSL112 as compared to the prototype formulation did not demonstrate a hepatic safety concern. Furthermore, infusion of CSL112 shortly after a contrast load among MI patients was not associated with renal toxicity, demonstrating the feasibility of administering CSL112 to MI patients with normal renal function or mild renal impairment shortly after angiography. A study in MI patients with moderate renal impairment is ongoing.

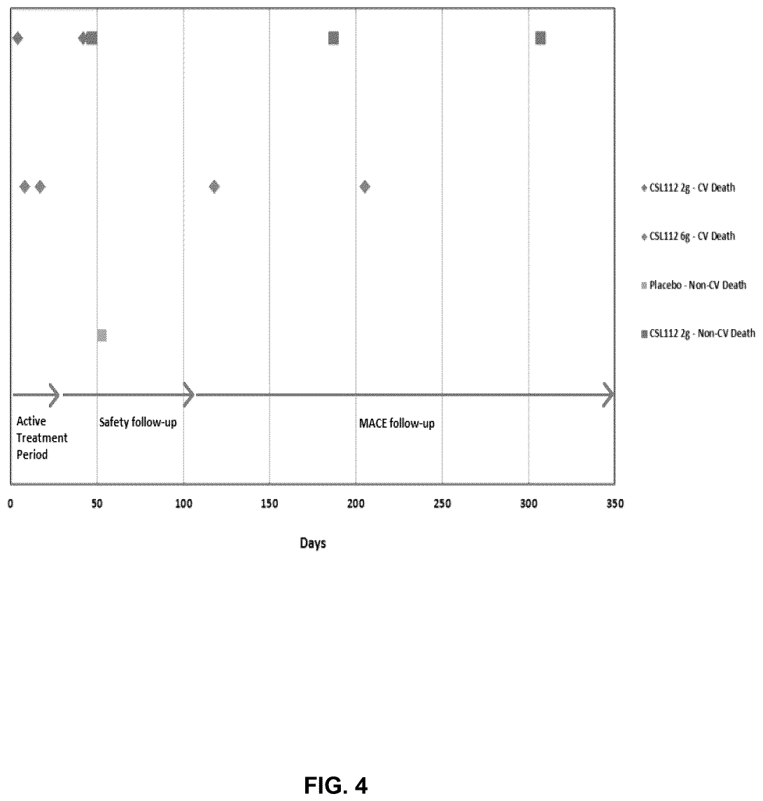

The number of MACE events overall was low (n=74) as was the number of subjects with complete follow-up through one year (89/1258). The statistical power to assess the secondary MACE endpoint was very low, approximately 8.4% (Table 13). MACE rates were generally comparable between groups, although cardiovascular mortality was higher in the 6 g group compared to placebo (4 vs 0 deaths, p=0.0477). The calculated p-value was not adjusted for the multiplicity of 32 efficacy comparisons. There was no clustering of death in proximity to the CSL112 infusion (Table 12 and FIG. 4). It should be noted that indeterminant causes of death were included as cardiovascular death. The isolated difference in mortality was inconsistent with the overall similarity in MACE rates.

Compared with placebo, CSL112 was also associated with an improvement in measures of cholesterol efflux capacity. It has been postulated that improvements in HDL function, rather than HDL concentration, may be more important for the stabilization of atherosclerotic plaque lesions and the reduction of CV events. In the Dallas Heart Study, high cholesterol efflux capacity, a marker of effective reverse cholesterol transport, was associated with a 67% lower risk of MACE as compared with low cholesterol efflux capacity¹⁸, an association that was independent of HDL concentrations. To date, while HDL-raising therapies have indeed increased HDL concentrations, they have had a modest or no effect on cholesterol efflux, a finding which may explain at least in part why HDL-raising therapies have failed to reduce MACE outcomes in the past³²⁻³⁸. In contrast, cholesterol efflux capacity was markedly elevated immediately following CSL112 infusion. In particular, ABCA1-dependent efflux, a pathway especially relevant to cholesterol-laden cells in plaque, was elevated more than three-fold after infusion of CSL112. It is noteworthy that the elevation in ABCA1-dependent efflux capacity was greater than the elevation of apoA-I thus suggesting that infusion elevates not just the quantity but also the functionality of the apoA-I pool. Indeed, the ratios of ABCA1-dependent cholesterol efflux capacity/apoA-I were elevated with both doses of CSL112 compared to placebo (Table 6). Prior mechanistic studies 39 have shown comparable functional changes and have determined that CSL112 elevates ABCA1-dependent efflux by remodeling endogenous HDL to form smaller, more functional HDL species with high ability to interact with ABCA1.

The elevation of cholesterol efflux caused by CSL112 has been shown to be transient and recedes to baseline with clearance of the apoA-I²⁶. It is not known how a transient enhancement of cholesterol efflux capacity immediately following acute MI will impact clinical outcomes as compared to the sustained or long term measures of cholesterol efflux assessed in the Dallas Heart Study¹⁸. Although MACE events were not reduced in AEGIS-I, this Phase 2b study was designed as a safety trial and was not sufficiently powered to assess efficacy (Table 13). Consistent with other Phase 2 safety studies, major adverse cardiovascular events (MACE) was explored in AEGIS-I to assess the timing and frequency of events and to identify subgroups of patients at higher risk of events so that an adequately powered phase 3 study could be planned to definitively assess the efficacy. Even though these analyses are exploratory, they were pre-specified so as to focus the analyses for phase 3 planning.

The co-primary safety endpoints were less frequent than anticipated for the non-inferiority analysis, but the very low frequency of these events suggests that there is not a clinically relevant hepatic or renal safety signal. Although several lipid and lipoprotein analyses were performed, Lp(a) and apoE were not assessed post infusion.

This was a Phase 2 safety study that was underpowered to assess efficacy and was not designed to seek regulatory approval for efficacy. For the secondary MACE endpoint, the power was 8.4% to detect a clinically relevant 15% risk reduction assuming a placebo event rate of 5.5% (Table 13). Like many Phase 2 studies, this trial was primarily undertaken to assess safety but also to assess the frequency and timing of MACE and to identify patients at risk for events so that an adequately powered pivotal phase 3 trial could be undertaken to assess efficacy.

In conclusion, 4 weekly infusions of CSL112, a reconstituted plasma-derived apoA-I, at both low [2 g] and high [6 g] doses beginning within 7 days of acute MI and in proximity to contrast media administration, were feasible, were not associated with alterations in either liver or kidney function or other significant safety concern, and were associated with acute enhancements in cholesterol efflux capacity. Further assessment of the clinical efficacy of CSL112 for the reduction of early recurrent cardiovascular events following acute MI is warranted in an adequately powered, multicenter, randomized phase 3 trial.

### Example 2

### Introduction

This example describes clinical study data of CSL112 and its ability to efflux cholesterol from macrophages in patients with moderate renal impairment.

Previous clinical studies with CSL112 have demonstrated favourable safety, pharmacokinetic (PK) and pharmacodynamics responses in healthy subjects, patients with stable atherosclerotic disease and acute MI patients with normal renal function (NRF) or mild renal impairment^{26,27}. Renal impairment is a prevalent concurrent condition in acute coronary syndrome, with approximately 30% of subjects having Stage 3 chronic kidney disease (CKD). The aim of the study was to assess the impact of CSL112 infusion on CEC and lipoprotein biomarkers in subjects with moderate renal impairment (Mod RI).

### Reverse Cholesterol Transport

In reverse cholesterol transport, free cholesterol (FC) is transferred from cells to pre-β1-HDL via the ABCA1 transporter, which is abundantly expressed on plaque macrophages in atherosclerotic lesions. FC in the HDL particle is then esterified by lecithin-cholesterol acyltransferase (LCAT) forming larger HDL particles (HDL3 and HDL2). FC is also transferred to HDL3 via the ABCG1 and SR-B1 transporters. Esterified HDL cholesterol is then transferred to the liver for excretion or reutilisation.

Infusion of CSL112 increases the formation of pre-β1-H1DL, which in turn increases CEC, predominantly via the ABCA1 transporter, and ultimately increases LCAT activity and the esterification of FC.

### Methods

### Study Design

A Phase 1, double-blind, single ascending dose study (NCT02427035) was conducted to assess PK, safety and biomarkers of CSL112 in adults with Mod RI. Renal impairment was classified as moderate if the eGFR is ≥30 and <60 mL/min/1.73 m². This is compared to NRF where eGFR is ≥90 mL/min/1.73 m².

There were 32 subjects in total, including 16 with NRF and 16 with Mod RI. Subjects were randomized, by renal function group, to receive 2 g (n=6 per group) or 6 g (n=6 per group) of CSL112 or placebo (n=4 [n=2 per CSL112 dose group]).

The study consisted of a 28-day screening period, followed by a 16-day active treatment period that included a mandatory in-house stay, during which CSL112 was administered as a single 2 hour intravenous (IV) infusion, several outpatient visits, and a 76-day safety follow-up period.

### Biomarker Assessments

Thirteen different baseline cholesterol efflux and lipoprotein parameters were measured in each renal function group. Plasma apoA-I, apolipoprotein B (apoB) and high sensitivity C-reactive protein (hsCRP) were measured by an immunoturbidimetric method. CEC, total and ABCA1-independent, was measured after incubation of serum in vitro with macrophages preloaded with radiolabelled cholesterol, not expressing ABCA1 or with ABCA1 expression induced by cyclic AMP (see, e.g., de le Llera-Moya et al., Arterioscler. Thromb. Vasc. Biol. 2010; 30-796-801). ABCA1-dependent CEC was calculated by subtraction of ABCA1-independent CEC from total CEC. Pre-j31-HDL was measured using a sandwich ELISA employing a conformational-specific antibody to apoA-I within pre-β1-HDL. Other lipid parameters were assessed by standard enzymatic methods.

### Statistical Analysis

A parallel t-test was used to compare baseline cholesterol efflux and lipoprotein parameters between patients with Mod RI and NRF. Biomarker exposures over CSL112 dose were compared between renal function groups by ANOVA.

### Results

### Baseline Characteristics

In total, 32 subjects (n=16 NRF and n=16 Mod RI) received a single IV infusion of CSL112 or placebo. The baseline characteristics of each of these patient groups is shown in Table 14.

At baseline levels, total and ABCA1-dependent CEC were 1.3-fold and 1.8-fold higher, respectively, in Mod RI subjects compared to subjects with NRF, but there was no significant difference in ABCA1-independent CEC. Consistent with this finding was a significant 1.4-fold increase in baseline pre-(31-HDL in the Mod RI group compared to the NRF group. All other lipid and lipoprotein levels and hsCRP were similar between renal function groups at baseline (Table 15). (Meier et al., Life Sci 2015; 136:1-6, previously observed a higher CEC at lower eGFR in adult CKD patients ( ).

All other lipid and lipoprotein levels and hsCRP were similar between renal function groups at baseline. (Table 15). Infusion of CSL112 did not significantly alter levels of proatherogenic lipids apoB, non-HD cholesterol or triglycerides, from baseline levels, in either renal function group (data not shown).

### Cholesterol Efflux and Lipoprotein Parameters Upon CSL112 Infusion

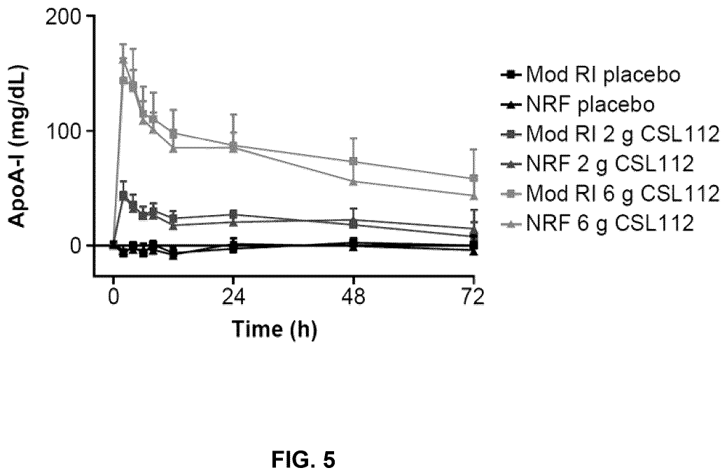

Following infusion of CSL112, ApoA-I rapidly increased in a dose-dependent manner, peaked at the end of the infusion period (2 h), and remained elevated above baseline levels at 72 h post-infusion. Plasma ApoA-I concentrations over time were similar between renal function groups, within each CSL112 dose group (FIG. 5).

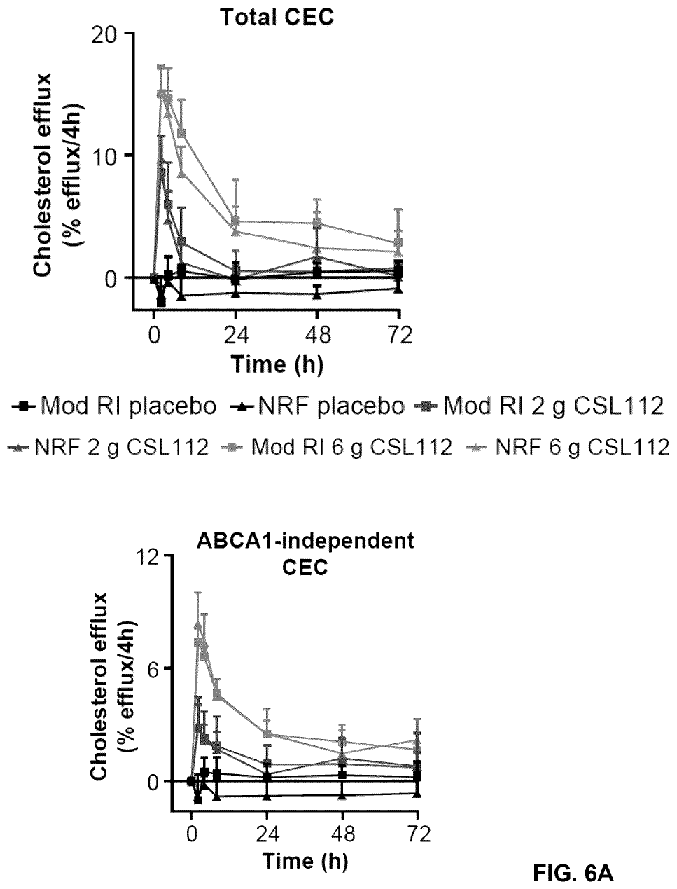

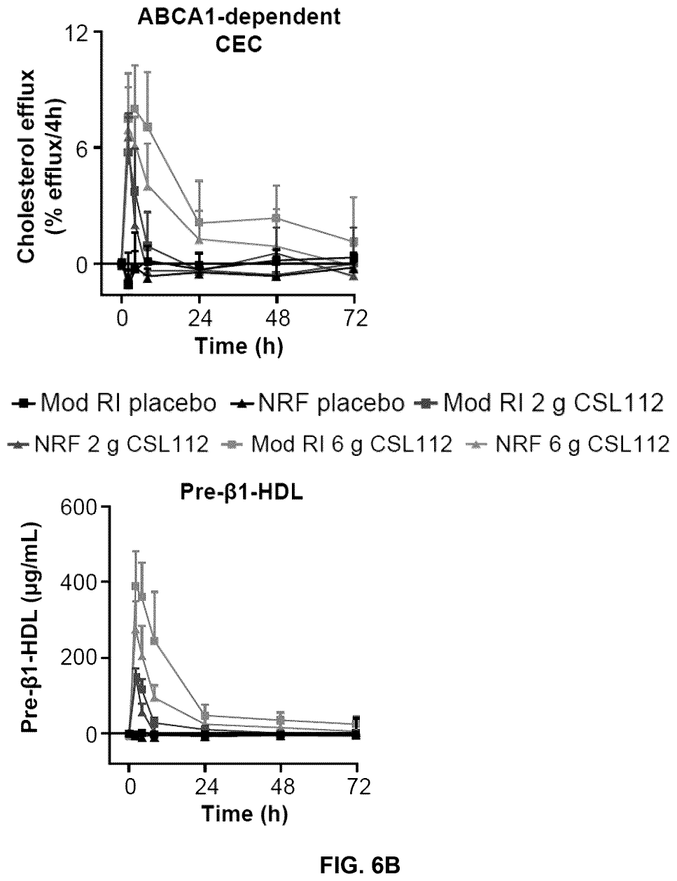

Rapid dose-dependent increases in total, ABCA1-dependent and ABCA1-independent CEC were observed following CSL112 infusion. The impact of CSL112 infusion on total and ABCA1-independent CEC was similar between renal function groups. In both renal function groups, CSL112 dose-dependently increased pre-β1-HDL levels (FIG. 6A-B).

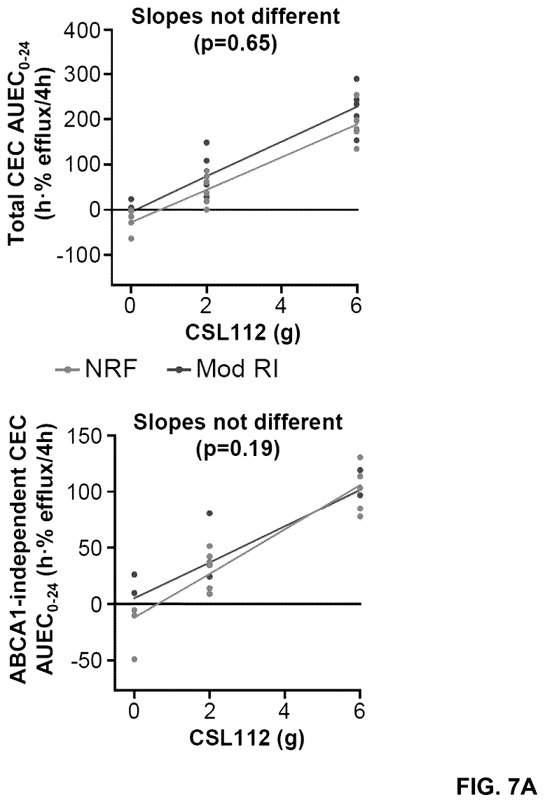

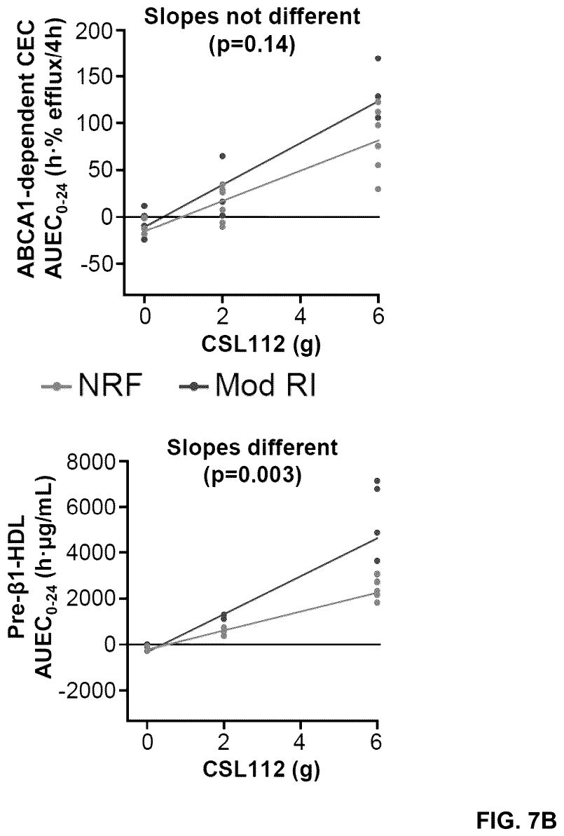

In both renal function groups, CSL112 dose-dependently increased total CEC, ABCA1-independent CEC, ABCA1-dependent CEC and pre-β1-HDL levels. For pre-β1-HDL, this dose-dependent increase was greater for subjects with Mod RI compared with subjects with NRF (FIG. 7A-B).

Without being bound by theory, a possible explanation for this finding is downregulation of expression of ABCA1 on peripheral cells in subjects with Mod RI leads to an increase in pre-β1-HDL due to a reduced ability to metabolize pre-β1-HDL to HDL3. In this case, CSL 112 infusion would lead to a more robust increase in pre-β1-HDL in Mod RI subjects compared with subjects with NRF. This is consistent with the baseline difference in pre-(31-HDL (Table 14).

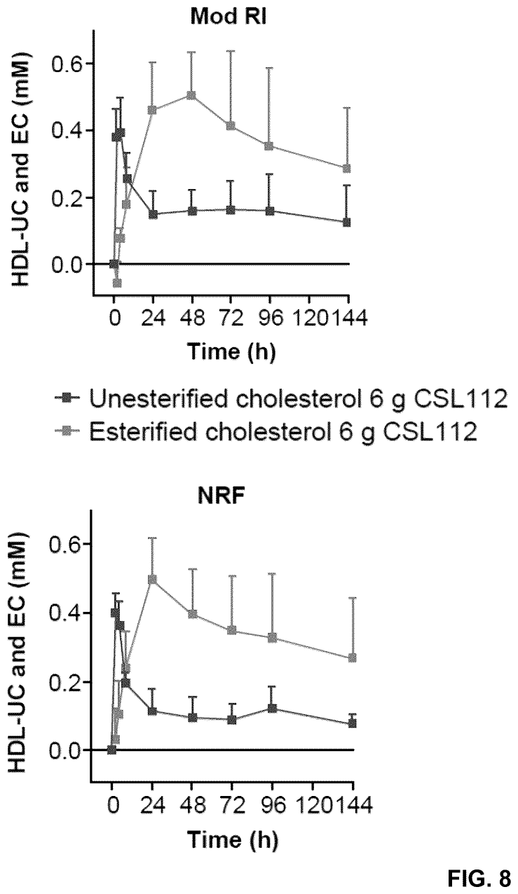

Following infusion of CSL112, there was a transient dose-dependent increase in HDL-unesterified cholesterol levels (HDL-UC), which peaked at the end of the infusion (2 h) and then declined (FIG. 8). This was followed by an increase in HDL-esterified cholesterol (HDL-EC), peaking at 24-h post-infusion and exceeding the level of HDL-UC. Both HDL-UC and HDL-EC levels were sustained above baseline levels at 144-h post infusion. Similar findings were seen with CSL112 at doses of 2 g. This finding is consistent with continuous movement of unesterified cholesterol into HDL and rapid esterification by LCAT. LCAT activity was not directly measured in this study but a strong rise in esterification was previously observed in plasma from rabbits infused with CSL112. Within dose groups, CSL112 had a similar impact on levels of HDL-UC and HDL-EC in both renal function groups. (FIG. 8) Infusion of CSL112 did not significantly alter levels of pro-atherogenic lipids apoB, non-HDL cholesterol or triglycerides, from baseline levels, in either renal function group.

### Conclusions

Infusion of CSL 112 in subjects with Mod RI and NRF resulted in similar immediate, robust, dose-dependent elevations in apoA-I and CEC. Mod RI subjects had greater elevations in pre-β1-HDL (p=0.003) which may reflect a reduced ability to metabolize pre-31-HDL to HDL3. LCAT activity, depicted by a time-dependent change of the ratio of free cholesterol to esterified cholesterol, appeared similar in Mod RI and NRF subjects. No changes from baseline were observed in association with CSL112 in apoB, non-HDL cholesterol, or triglycerides concentrations in either group.

This study data shows that CSL112 enhances biomarkers of reverse cholesterol transport similarly in subjects with Mod RI and NRF. This indicates that CSL112 may provide a novel therapy to rapidly lower the burden of atherosclerosis and to reduce the risk of recurrent cardiovascular events in patients with and without Mod RI following acute myocardial infarction.

These results were obtained in Mod RI subjects who had not experienced an MI event within seven days prior to starting treatment.

### Example 3

### Introduction

In patients with ACS and RI, the prognosis, both short- and long-term, is worse than for those with normal renal function, as the risk of CV events and mortality is inversely proportional to the estimated glomerular filtration rate (eGFR) [Nabais et al, 2008; Bhandari and Jain, 2012]. As subjects with moderate RI present a significant portion (ie, up to 30% [Gibson et al, 2004; Fox et al, 2010]) of the ACS population, it is important to include this subpopulation in the CSL112 phase 3 program.

Study CSL112_2001, a phase 2, multicenter, double-blind, randomized, placebo-controlled, parallel-group, study was undertaken to evaluate the renal and other safety of multiple dose administration of CSL112 6 g in subjects with AMI and moderate RI.

### Study Design

Study CSL112_2001 enrolled subjects with moderate RI who were screened within 5 to 7 days of experiencing an AMI. Approximately 81 subjects were to be enrolled and randomly assigned to receive 4 weekly infusions of 6 g CSL112 (˜54 subjects) versus placebo (˜27 subjects) to evaluate renal and other safety parameters. To ensure that at least one-third of the study population had an eGFR in the chronic kidney disease (CKD) stage 3b range (eGFR 30 to <45 mL/min/1.73 m²), no more than two-thirds of the study population (ie, 54 subjects) were to have an eGFR in the CKD Stage 3a range (45 to <60 mL/min/1.73 m²). Randomization was stratified by eGFR (30 to <45 mL/min/1.73 m² or 45 to <60 mL/min/1.73 m²) as calculated by the Chronic Kidney Disease Epidemiology (CKD-EPI) equation [Levey et al, 2009; Stevens et al, 2010], and by medical history of diabetes with current pharmacotherapy. Subjects were to be followed for approximately 60 days.

### Study Objectives and Endpoints

The primary objective of study CSL112_2001 was to assess the renal safety of CSL112 in subjects with moderate RI and AMI. Co-primary endpoints were the incidence of renal SAEs and AKI events. Incidence rates were based on the number of subjects with at least 1 occurrence of the event of interest.
Renal SAEs were defined by Medical Dictionary of Regulatory Activities (MedDRA) preferred term (PT) included in the Acute Renal Failure narrow Standard MedDRA Query (SMQ) or a PT of Renal Tubular Necrosis, Renal Cortical Necrosis, Renal Necrosis, or Renal Papillary Necrosis.
Acute kidney injury was defined as an absolute increase in serum creatinine from baseline ≥0.3 mg/dL (26.5 μmol/L) during the Active Treatment Period that was sustained upon repeat measurement by the central laboratory no earlier than 24 hours after the elevated value. If no repeat value was obtained (due to loss of follow-up or protocol violation, for example), a single serum creatinine value that was increased from baseline ≥0.3 mg/dL (26.5 μmol/L) during the Active Treatment Period would also fulfill the definition of AKI. Baseline for determination of AKI was defined as the pre-infusion central laboratory serum creatinine level on Study Day 1.

Secondary objectives of the study were 1) to further characterize the safety and tolerability of CSL112 in subjects with moderate RI and AMI and 2) to characterize the PK of CSL 112 after multiple dose administration in subjects with moderate RI and AMI.

The corresponding endpoints for these objectives included:
Incidence of TEAEs and adverse drug reactions (ADRs) or suspected ADRs
Incidence of treatment-emergent bleeding events
Change from baseline in renal (serum creatinine, eGFR) and hepatic function (alanine aminotransferase [ALT], total bilirubin) tests
Clinically significant changes in clinical laboratory tests results (serum biochemistry, hematology, and urinalysis), physical examinations findings, body weight, electrocardiograms (ECGs), and vital signs
Occurrence of antibodies to CSL112 or apoA-I
Plasma concentration at baseline and End-of-Infusion for apoA-I and PC
Accumulation ratio (R) for apoA-I and PC

Exploratory objectives of the study were to 1) characterize the pharmacodynamic features of CSL112 by evaluating cholesterol efflux and other lipid and CV biomarkers of CSL112 activity, and 2) assess the effect of CSL112 on renal safety biomarkers.

### Results

### Subject Disposition

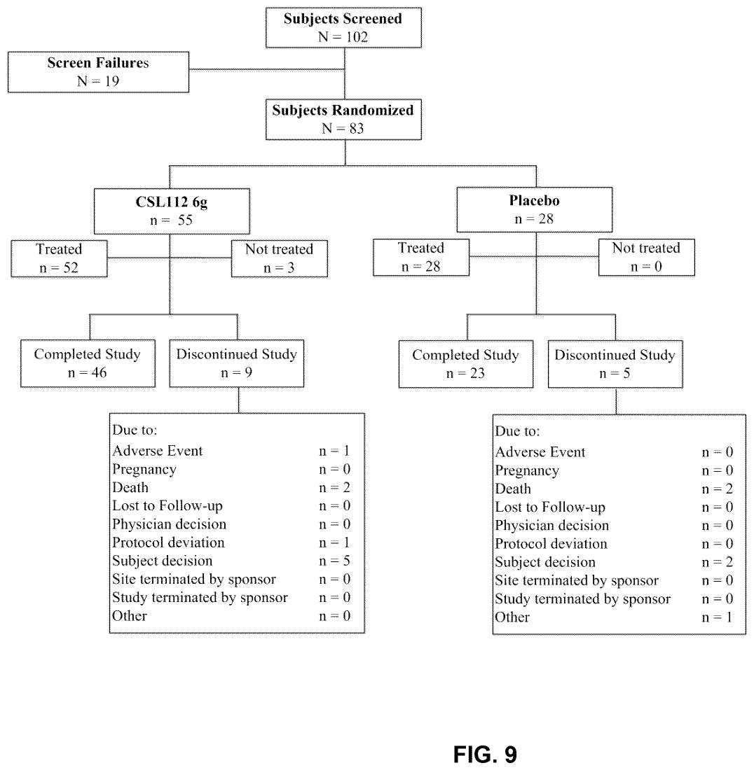

A total of 102 subjects provided written informed consent and were screened for inclusion in study CSL112_2001 (FIG. 9). Of these subjects, 19 were screen failures, and the remaining 83 (81.4%) eligible subjects were randomized 2: 1 active to placebo to receive 6 g of CSL112 (55 subjects, 53.9%) or placebo (28 subjects, 27.5%), respectively. Three subjects who were randomized to CSL112 did not receive treatment. Sixty-nine (83.1%) randomized subjects completed the study, with 46 (83.6%) subjects completing in the CSL112-group and 23 (82.1%) subjects completing in the placebo group.

Fourteen (16.9%) subjects did not complete the study, 9/55 (16.4%) and 5/28 (17.9%) in the CSL112 and placebo groups, respectively. Reasons for subjects not completing the study included AEs (1.8% CSL112; 0 placebo), death (3.6% CSL112; 7.1% placebo), protocol deviation (1.8% CSL112; 0 placebo), subject decision (9.1% CSL112; 7.1% placebo), and other (0 CSL112; 3.6% placebo).

### Baseline Characteristics

The subject mean age was 71.1 years, with 81.9% of subjects at least age 65 years, and with a mean BMI of 29.5 kg/m². The treatment groups were well-balanced for both age and sex (Table 16).

Subject mean eGFR at screening was 46.32 mL/min/1.73 m² as determined by the central laboratory. Median eGFR laboratory values approximated the chronic kidney disease (CKD) stage 3a/3b cut point (ie, 45 mL/min/1.73 m²). At randomization, 47.0% and 53.0% of subjects were classified based on local laboratory assessment as having CKD stage 3b (30 to <45 mL/min/1.73 m²) or stage 3a (45 to <60 mL/min/1.73 m²), respectively, with central laboratory data categorizing 39.8% of subjects having CKD Stage 3b and 44.6% having CKD Stage 3a. Variation in the assays between the central and local laboratories may have contributed to the re-categorization of subjects based on central laboratory results as compared to local laboratory results which were used for randomization.

Subjects were receiving aspirin (95.2%), other anti-platelet drugs (91.6%), statins (89.2% overall; 59.0% high intensity), other lipid modifying agents (6.0%), beta-blockers (79.5%), angiotensin I converting enzyme inhibitors or angiotensin receptor blockers (74.7%), and oral anti-thrombotics (26.5%).

Overall, the treatment groups were well-balanced for demographic and baseline characteristics.

### Analysis of Safety

### Study-Drug Exposure

All 80 (100%) subjects in the safety population completed at least 1 infusion of study drug; most subjects (81.3%) received and completed 3 or 4 infusions of study drug.

A total of 55/80 (68.8%) subjects in the safety population completed all 4 infusions. Reasons for subjects not completing all 4 infusions included AEs (19.2% CSL112; 14.3% placebo), subject decision (5.8% CSL112; 10.7% placebo), death (1.9% CSL112; 3.6% placebo), key renal values (0 CSL112; 3.6% placebo), physician decision (1.9% CSL112; 0 placebo), and other (1.9% CSL112; 0 placebo).

Investigational product was discontinued in 4 subjects due to a renal-related adverse event, 3 (3.8%) and 1 (3.4%) subjects in the CSL112 6 g and placebo groups, respectively. In the CSL112 6 g group, all events were assessed as not related by the investigator. Two events in 2 subjects were non-serious and each subject received 3 doses of CSL112. The third subject had an SAE of nephropathy toxic on study day 2 after receiving 1 dose of CSL112. In the placebo group, 1 subject had an SAE of renal failure on study day 12 and received 2 doses of placebo. This event was assessed as related to IP by the investigator. One subject in the CSL112 group had an infusion skipped due to “blood creatinine increased” and 2 subjects in the placebo group had an infusion skipped, 1 due to “acute kidney injury” and 1 due to meeting a key renal laboratory value defined by the individual subject dose delay and stopping rules that was not assessed as an adverse event.

Two subjects in the CSL112 group had hepatic AEs (ALT increased, total bilirubin increased; both mild and transient) that met protocol criteria for discontinuation of study drug; no subjects in the placebo group discontinued due to hepatic reasons.

### Timings Up to First Infusion of Study Drug

The mean time elapsed between angiography and the first infusion of study drug was 65.2 hours (2.7 days), with the elapsed time slightly shorter for the CSL 112 6 g (61.83 h [2.57 days]) group versus the placebo (71.79 h [2.99 days]) treatment group. The mean time elapsed between angiography and the first infusion was 59.47 hours (2.48 days) for subjects with their MI classified as STEMI versus 67.2 hours (2.8 days) for those classified as NSTEMI. Similar percentages of STEMI (40.0%) and NSTEMI (38.6%) subjects were dosed with study drug within less than 48 hours after contrast administration. A low percentage (5/77, 6.5%) of subjects received the first infusion within 12 to <24 hours of angiography (Table 17).

### Co-Primary Endpoint

A summary of treatment-emergent renal SAEs and AKI events is provided in Table 18.

Treatment-emergent renal SAEs were reported for 1/52 (1.9%) subjects in the CSL112 6 g treatment group compared with 4/28 (14.3%) subjects in the placebo group. Based on the primary analysis, the difference in incidence rates (95% confidence interval) between these treatment groups was −0.124 (−0.296, −0.005). All subjects with renal SAEs experienced 1 event, except for 1 subject in the placebo group who experienced 2 events.

Treatment-emergent AKI events, were reported for 2/50 (4.0%) subjects in the CSL112 6 g treatment group as compared with 4/28 (14.3%) subjects in the placebo group. Based on the primary analysis, the difference in incidence rates (95% confidence interval) between these treatment groups was −0.103 (−0.277, 0.025). There were no subjects with more than 1 AKI event. For the 6 subjects with AKI events, these events were ongoing at study completion. Within both groups of subjects based on time between contrast and serum creatinine determination, the observed rate of AKI was numerically smaller in the CSL112 group compared with the placebo group (Table 18).

Sensitivity analysis of the co-primary endpoints using independently adjudicated results for the treatment-emergent renal SAE component and local laboratory data for the treatment-emergent AKI component support results of the primary analysis.

There was no indication that the rate of renal SAEs or AKI events was greater in the CSL112 group relative to placebo in subjects within the CKD Stage 3a or 3b subgroups or in subjects with diabetes. Within these subgroups, higher rates of renal SAEs and AKI events were observed in the placebo group (Table 19). There was a higher rate of AKI events in the CSL112 group for subjects without a history of diabetes.

### Adjudicated Renal Serious Events

Investigator-identified renal serious events were adjudicated by the clinical events committee and of the 6 investigator reported events, 5 were positively adjudicated: ½ in the CSL112 group and 4/4 in the placebo group. One event in the CSL112 group was adjudicated as not being an event as it was not serious.

All events were classified as non-obstructive (i.e. not due to a physical obstruction in the kidney or ureter, such as a kidney stone) and the causality for events was possible for 1 event in the CSL112 group and possible or unlikely for 3 and 2 events, respectively, in the placebo group. At the time of diagnosis all events were Stage 1. Progression to Stage 2 occurred for the single positively adjudicated event in the CSL112 group within 7 days of the start of the AKI event; for the placebo group, 2 events progressed within this time frame, 1 each to Stage 2 (25%) and Stage 3 (25%).

### Adverse Events

Unless otherwise stated, all AEs described in this section refer to TEAEs.

### Overall Summary

An overall summary of TEAEs discussed herein is presented in Table 20.

### Treatment-Emergent Adverse Events

Similar percentages of subjects in the CSL112 and placebo groups reported treatment-emergent AEs (TEAEs): 38 (73.1%) subjects in the CSL112 6 g group and 20 (71.4%) subjects in the placebo group. System organ classes with frequent (≥10%) TEAEs at a higher rate in the CSL112 group compared with placebo included: Cardiac disorders, Investigations, Respiratory, thoracic and mediastinal disorders, Gastrointestinal disorders, and Nervous system disorders.

Overall, similar percentages of TEAEs of CTCAE Grade 3, 4, and 5 in severity were reported for the CSL112 (17.3%, 7.7%, and 3.8%, respectively) and placebo (35.7%, 3.6%, and 7.1%, respectively) groups. There were 15/52 (28.8%) subjects in the CSL112 group who experienced a Grade 3, 4 or 5 TEAE, compared to 13/28 (46.4%) subjects in the placebo group. Grade 5 events occurred at higher frequency in the placebo group (2/28, 7.1%) compared with the CSL112 group (2/52, 3.8%). Frequent (≥10% or more of subjects) TEAEs that occurred in the CSL112 group alone included Blood creatinine increased, Cardiac failure, and Atrial fibrillation.

### Serious Adverse Events

A total of 22/80 (27.5%) subjects experienced serious TEAEs, with 12/52 [23.1%] and 10/28 [35.7%] in the CSL112 6 g and placebo groups, respectively (Table 21). Serious TEAEs were reported among the following SOCs: Cardiac disorders (12.5%), Urinary and renal disorders (6.3%), Infections and infestations (3.8%), Gastrointestinal disorders, General disorders and administration site conditions, Injury, poisoning and procedural complications, Nervous system disorders, and Respiratory, thoracic and mediastinal disorders (2.5% each), Blood and lymphatic system disorders, Ear and labyrinth disorders, Eye disorders, and Vascular disorders (1.3% each).

Serious TEAEs reported for 2 or more subjects in the CSL112 group included (by preferred term) Atrial fibrillation (3/52, 5.8%) and Cardiac failure (3/52, 5.8%). For subjects in the placebo group, serious TEAEs reported for 2 or more subjects included Cardiac failure congestive (2/28, 7.1%) and AKI (2/28, 7.1%).

### Heart Failure and all Renal Events of Interest

Adverse events that were evaluated in more detail include heart failure and all renal events.

Treatment-emergent adverse events of heart failure that were reported included, by preferred term: Cardiac failure, Cardiac failure congestive, and Cardiac failure acute. A higher percentage of subjects in the CSL112 (7/52, 13.5%) group compared with the placebo (2/28, 7.1%) group had TEAEs of heart failure. Treatment-emergent SAEs of heart failure occurred at a similar frequency in the CSL112 (4/52, 7.6%) and placebo (2/28, 7.1%) groups. One subject in each of the CSL112 and placebo groups had an event of heart failure that resulted in death.

Treatment-emergent renal events included by preferred term: Renal failure, Nephropathy toxic, AKI, Renal impairment, and Blood creatinine increased. These events occurred at similar rates for subjects in the CSL112 (17.3%) and placebo (14.3%) groups. As noted previously (see Co-primary Endpoint), treatment-emergent renal SAEs occurred at a lower rate for subjects in the CSL112 group (1.9%) compared with the placebo group (14.3%).

### Treatment-Emergent Bleeding Events

Treatment-emergent bleeding events were reported by investigators and adjudicated by the clinical events committee based on the Bleeding Academic Research Consortium (BARC) criteria. Similar rates and severity of bleeding events were observed in each treatment group. Among subjects who experienced a bleeding event, all were BARC Grade 3 or below. A total of 3/52 (5.8%) subjects in the CSL112 6 g group experienced BARC Grade Type 3 bleeds compared with 1/28 (3.6%) in the placebo group. No subjects in either treatment group experienced a BARC Grade Type 4 or 5 event. There were no deaths related to bleeding events and there were no central nervous system bleeds

### Adverse Drug Reactions or Suspected Adverse Drug Reactions

Treatment-emergent AEs classified as ADRs or suspected ADRs based on the FDA definition¹ were at a higher frequency in the CSL112 group (57.7%) compared with the placebo group (14.3%).

The classification of a large percentage of TEAEs in the CSL112 group, as suspected ADRs is due to applying the 4-part FDA definition to a study with a small sample size. According to the fourth criterion, if 1 subject in an active treatment arm and no subjects in the placebo arm had an event, the event would be classified as a suspected ADR. Given the small sample size, there are inadequate data to determine if all TEAEs that were reported in the study are ADRs (i.e. causally related to CSL112).

### Clinical Laboratory Test Results

### Changes in Renal Function Tests

In addition to the clinical events committee evaluation of the stage of renal SAEs, laboratory values were analyzed for elevations that would meet Kidney Disease: Improving Global Outcomes definitions of AKI (KIDGO, 2012). No subjects in the CSL112 or placebo group experienced a Stage 3 AKI event (serum creatinine ≥3×the Baseline value or ≥4.0 mg/dL [353.6 [μmol/L]) based on central or local serum creatinine values (Table 22). Two subjects had missing central laboratory serum creatinine values at baseline. Most serum creatinine elevations (67.3% CSL112 6 g; 64.3% placebo) were in the range of ≥0 to <0.3 mg/dL increased from baseline. For each of these categories of absolute value increases from baseline in the range of ≥0.3 to ≤0.5 mg/dL and increases >0.5 mg/dL serum creatinine from baseline, a lower percentage of subjects were in the CSL112 6 g group compared with the placebo group. One (1.9%) subject in the CSL112 group and 4 (14.3%) in the placebo group had increases from baseline in serum creatinine in the range of ≥0.3 to ≤0.5 mg/dL sustained for ≥24 hours. One (1.9%) subject in the CSL112 6 g had a serum creatinine level >0.5 mg/dL sustained for ≥24 hours. No subjects had serum creatinine values ≥2-fold baseline values.

### Changes in Liver Function Tests

Mean values at baseline for alanine aminotransferase (ALT), aspartate aminotransferase (AST), alkaline phosphatase (ALP), and total and direct bilirubin were similar for both the placebo and CSL112 6 g groups. These parameters were not elevated after infusion of CSL112.

The percentage of subjects in either the placebo or CSL112 6 g groups who had missing values for ALT or total bilirubin was low. Across visits, the maximal percentage of subjects with missing values was 7.5% for both ALT and total bilirubin.

No subjects in either the CSL112 6 g or placebo groups had concomitant elevations in total or direct bilirubin greater than 2×ULN and ALT or AST greater than 3×ULN during the Active Treatment Period (Table 22). There were no subjects with elevations in ALT >3×ULN during the Active Treatment Period. One (1.9%) subject in the CSL112 group had an isolated increase in AST >5×ULN at Visit 3 that resolved by Visit 4. During the Active Treatment Period, 3 (5.8%) subjects in the CSL112 group had transient increases in total bilirubin (or direct bilirubin for subjects with Gilbert's syndrome) of >1.5×ULN at Visit 3, 24 to 48 hours after the start of infusion that were no longer present at Visit 4, compared with no subjects in the placebo group.

### Other Serum Biochemistry

No clinically meaningful differences in other serum biochemistry parameters were noted between treatment groups, and no clinically meaningful trends were observed overall.

### Hematology

No clinically meaningful differences in hematology parameters were noted between treatment groups, and no clinically meaningful trends were observed overall.

A total of 9/80 (11.3%) subjects had decreases in hemoglobin of ≥2 g/L from Baseline during the course of study with a higher percentage of subjects in the CSL112 6 g (7/52, 13.5%) compared with the placebo (2/28, 7.1%) group.

### Urinalysis

No clinically meaningful differences in urinalysis parameters were noted between treatment groups, and no clinically meaningful trends were observed overall. Shifts from baseline for hemoglobin and qualitative total protein in urine were few in number and for those shifts that did occur, it was by no more than 1 category. Spot urine protein/creatinine and urine cystatin C/creatinine ratios showed mild, transient increases in median values 24 to 48 hours after the first infusion of CSL112, with large variability in the data.

### Laboratory Abnormalities

No subject had Grade 4 laboratory abnormalities in hemoglobin, serum creatinine, eGFR, glucose (serum or urine), ALT, AST, ALP, or bilirubin (direct, indirect, or total). Grade 3 laboratory abnormalities were seen in subjects in both treatment groups for eGFR (3.8% CSL112; 7.4% placebo) and glucose (13.5% CSL112; 22.2% placebo). A single Grade 3 laboratory abnormality in AST was found in the CSL112 6 g group (see section: Changes in Liver Function tests).

### Immunogenicity

At baseline, all subjects had reciprocal antibody titers that were considered negative (10 or 11). No subjects in either the CSL112 6 g or placebo groups had a change from baseline in anti-CSL112 or anti-apoA-I reciprocal antibody titer at the end of the Active Treatment Period (Visit 7) or upon study completion (Visit 8).

### Analysis of Pharmacokinetics

Relative to baseline and to the placebo group, mean plasma concentrations for both apoA-I and PC were increased for the CSL112 group, with the highest mean values observed at the end of infusion 1 (Visit 2) and 4 (Visit 6) time points.

Similar increases in plasma concentrations of apoA-I and PC were observed for CSL112-treated subjects in each renal function subgroup at the end of infusion 1 (Visit 2) and 4 (Visit 6) time points.

Mean baseline-corrected maximal observed plasma concentration (Cₘₐₓ) values for apoA-I and PC were increased for the CSL112 6 g group relative to placebo after the first and fourth infusions (Table 24). The accumulation ratio for Cₘₐₓ values obtained after the 4^{th} infusion relative to the 1st infusion for apoA-I and PC were 1.20 (20%) and 1.00 (0%), respectively. For both CSL112 analytes, plasma accumulation was low.

The Total CEC was 13% higher (P<0.001) at baseline in the 2001 patients compared to the AEGIS-I patient population (Example 1). In particular the Total CEC % was 9.8+2.7 (n=78) for CSL112_2001 versus 8.7+2.7 (n=1204) for AEGIS-I. Similarly the ABCA1 dependent CEC was 35% higher (P<0.001) in the 2001 patients at baseline compared to the AEGIS-I patients. The ABCA1 dependent CEC % was 3.6±2.0 (n=78) for CSL112_2001 versus 2.6±1.8 (n=1204) for AEGIS-I. No difference was seen in the ABCA1 independent CEC with the ABCA1 independent CEC % being 6.2±1.7 (n=78) for CSL112_2001 versus 6.0±1.5 (n=1204) for AEGIS-I. These observations are consistent with the pattern of CEC observed in subjects with moderate RI versus normal renal function in the CSL112_1001 study (Example 2).

### Aggregate Renal Parameter Data: AEGIS I and CSL112_2001

Aggregate data analysis of changes from baseline in serum creatinine and eGFR is provided herein for the AEGIS-I (study CSLCT-HDL-12-77) and CSL112_2001 studies. The purpose of this data analysis was to ascertain the overall impact and the impact in relation to the timing of CSL112 infusion relative to angiography on renal function for subjects with various degrees of renal impairment. AEGIS-I evaluated CSL112 in MI subjects with either normal renal function or mild RI. Study CSL 112_2001 evaluated AMI subjects with moderate RI. Aggregate analysis of these data allows for evaluation across the spectrum of renal functions anticipated among the phase 3 target population. For both studies, enrolled subjects are representative of the target phase 3 population in age, sex, concurrent medical conditions (e.g. diabetes, hypertension) and chronic concomitant medications (e.g. dual anti-platelet therapy statins).

### Serum Creatinine

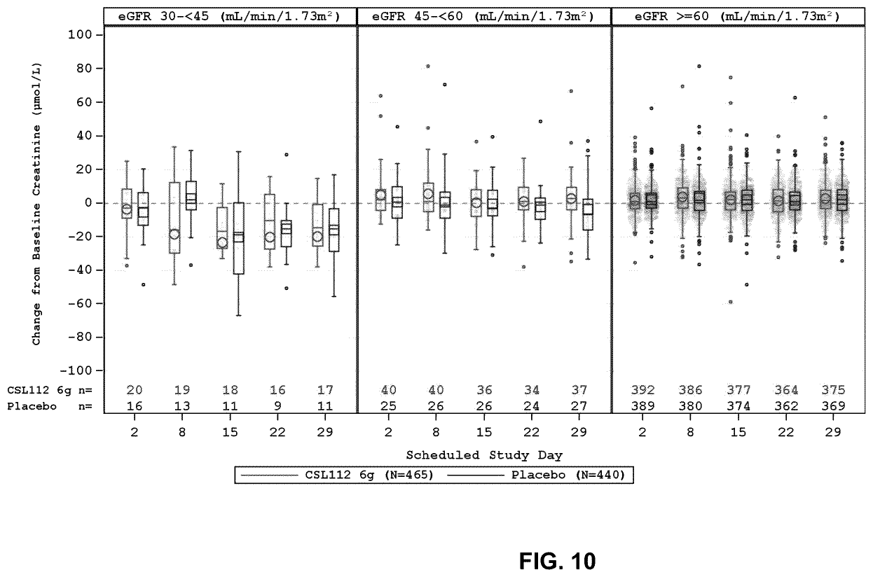

Aggregate analysis (FIG. 10) showed little change from baseline in mean serum creatinine levels for subjects treated with CSL112 or placebo with eGFR ≥60 mL/min/1.73 m² as well as for those subjects with eGFR 45-60 mL/min/1.73 m² during the Active Treatment Periods and out to 7 to 10 days following the last infusion. For subjects with eGFR 30-<45 mL/min/1.73 m² decreases from baseline in mean serum creatinine levels were observed for both treatment groups starting at study day 15. Relatively comparable decreases in mean serum creatinine levels were observed for subjects in the CSL112 and placebo groups.

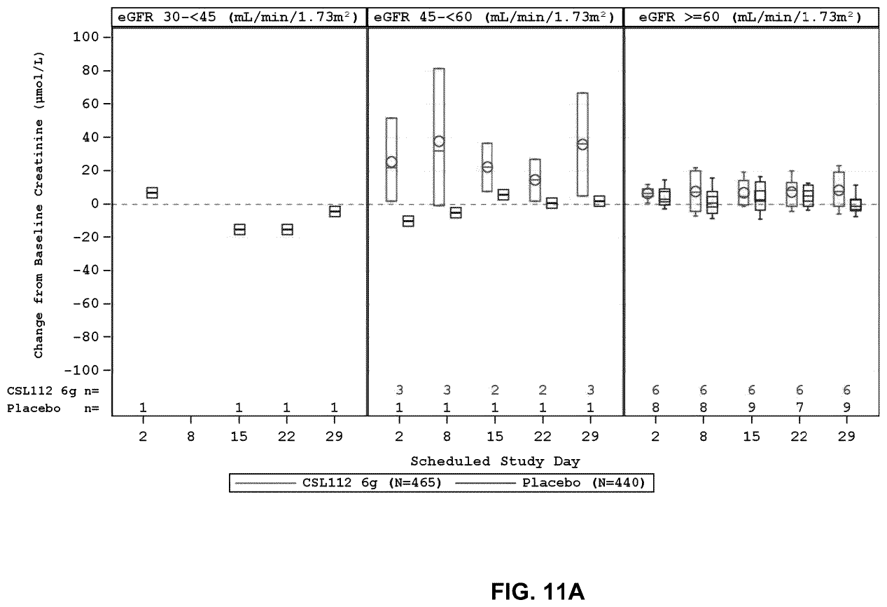

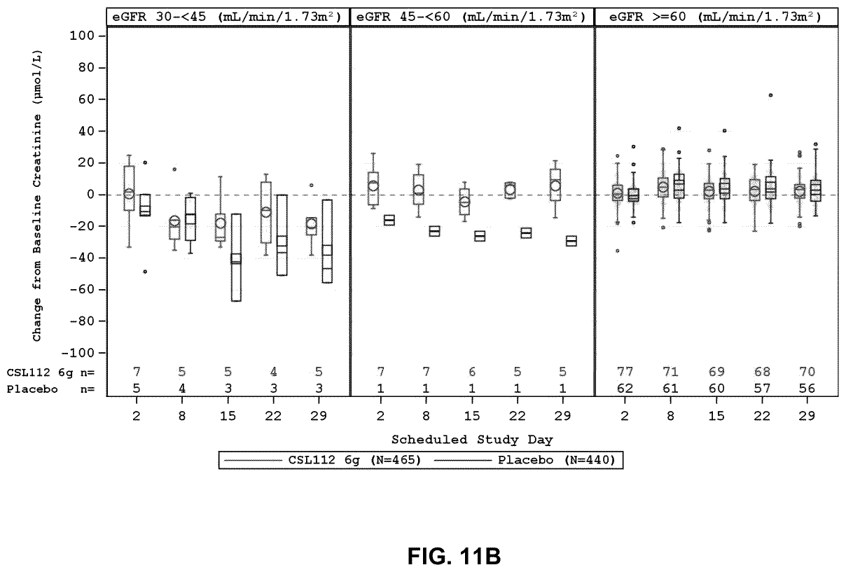

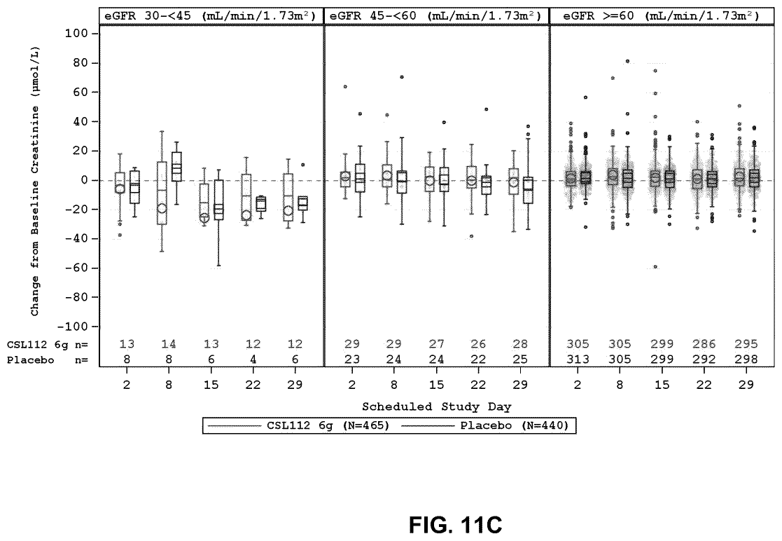

Analysis by renal stratum and time between angiography and first dose for change from baseline values (Central Laboratory) in serum creatinine showed decreases from baseline for subjects with eGFR in the range of 30 to <45 mL/min/1.73 m² in both the 24- to <48-hour window and the ≥48-hour window (FIG. 11). For subjects with an eGFR of 45 to <60 mL/min/1.73 m² dosed in the 24- to <48-hour window, for most subjects the change in creatinine was below 0.3 mg/dL increased from baseline. Data are insufficient to make conclusions for subjects dosed <24 hours after angiography.

### Estimated Glomerular Filtration Rate

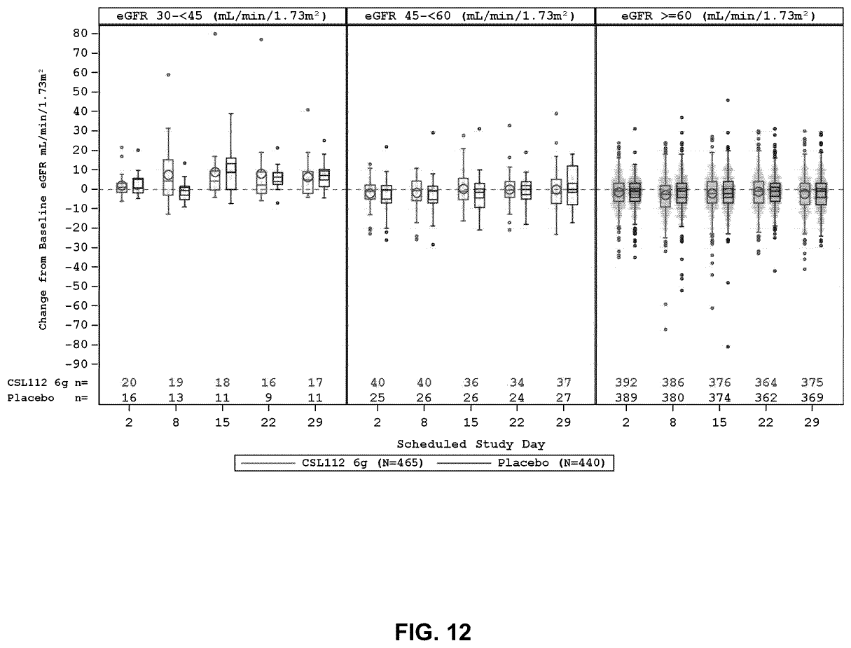

Aggregate analysis (FIG. 12) showed little change from baseline in eGFR for subjects with eGFR ≥60 mL/min/1.73 m² as well as for those subjects with eGFR 45-<60 mL/min/1.73 m² across the Active Treatment Periods and out to 7 to 10 days following the last infusion. For subjects with eGFR 30-<45 mL/min/1.73 m² small increases in the mean change from baseline in eGFR were observed for both CSL112- and placebo-treated subjects starting at study day 15. Summary tables of aggregate data for eGFR values are provided in FIGS. 10-12.

### Results

### Co-Primary Endpoints

The rate of renal-related serious and non-serious adverse events was similar between treatment groups (Table 19). There was no evidence of a higher rate of creatinine elevations with CSL112 treatment compared with placebo by either central or local laboratory analysis. Most creatinine elevations from baseline were mild and transient.

### Analysis of Adverse Events

Treatment-emergent AEs occurred in similar percentages of subjects in the CSL112 (73.1%) and placebo (71.4%) groups. There were no apparent imbalances in events within a SOC between treatment groups, and the most frequent AEs were expected based on the patient population of acute MI and moderate RI. There was a low frequency of related TEAEs, with 4 in the CSL112 group (ALT increase, blood bilirubin increase, infusion site swelling, and hyperventilation); there was 1 SUSAR of renal failure in the placebo group. No events of hemolysis occurred and similar rates and severity of bleeding were observed in both treatment arms. No fatal bleeds or central nervous system bleeds occurred during the course of the study.

### Hepatic and Other Laboratory Findings

Regarding hepatic findings, no subjects met Hy's Law criteria for drug-induced liver injury as no concomitant elevations in ALT >3×ULN and total bilirubin >2×ULN were observed for subjects in either treatment group. Mild, transient increases in total bilirubin or direct bilirubin for subjects with Gilbert's syndrome were observed in the 24 to 48 hours after the start of infusion 1 of CSL112 in a small percentage (5.8%) of subjects who received CSL112. These transient increases in indirect bilirubin have been seen previously in the program and are not considered clinically significant nor have they been associated with alterations in hepatic function.

Regarding other laboratory findings, no clinically meaningful differences were observed between treatment groups for hematology or biochemistry parameters. There were no safety findings with regards to total urine protein or clinically meaningful changes or differences between treatment groups in spot urine protein/creatinine ratios. No clinically meaningful differences between treatment arms were observed for serum cystatin C. No antibodies to CSL112 or apoA-I were detected.

### Pharmacokinetics

Pharmacokinetic evaluation demonstrated that there was no accumulation of apoA-I or PC with CSL112 treatment (4th infusion compared to 1st infusion) in subjects with acute MI and moderate RI, confirming the acceptability of the CSL112 6 g dose for use in this population. Similar elevations in apoA-I relative to baseline were observed in CSL112 treated subjects with CKD Stages 3a (eGFR=45-<60 mL/min/1.73 m²) and 3b (eGFR=30-<45 mL/min/1.73 m²).

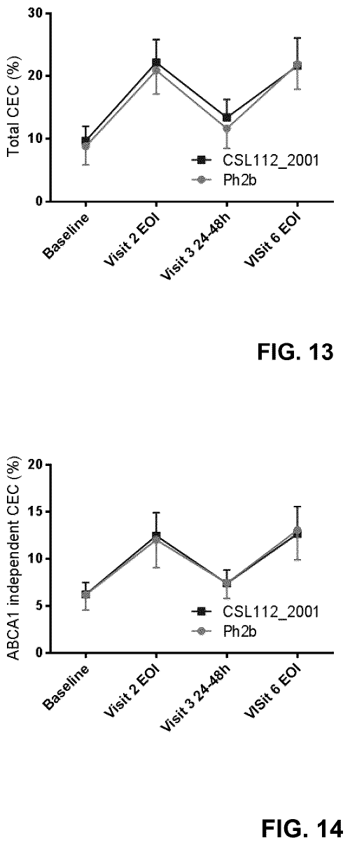

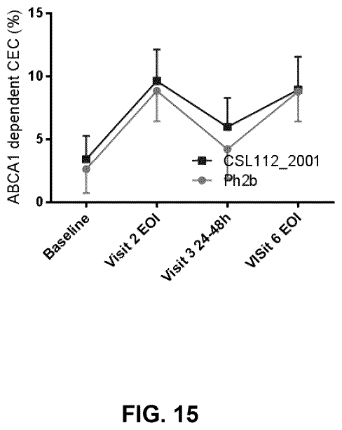

The study demonstrated that from a pharmacokinetic perspective the 6 g dose is appropriate for acute MI patients with moderate RI. The CSL112 6 g dose raised the CEC to a similar extent in the CSL112_2001 subjects compared to those in the AEGIS-I study (Example 1). At the end of infusion time points the relative increases in CEC were similar in both studies (FIGS. 13-15). The ABCA1 dependent CEC was elevated longer in the CSL112_2001 subjects which is consistent with that observed in the MRI patients receiving CSL112 in the CSL112-1001 study (Example 2).

### Aggregate Laboratory Analysis

An aggregate laboratory data analysis from studies AEGIS-I and CSL112_2001 examined changes from baseline in serum creatinine and eGFR and showed no negative impact of CSL112 infusion on these renal function parameters in subgroups of subjects with moderate RI when compared with mild RI or normal renal function. Changes from baseline in serum creatinine were similar across renal function groups regardless of the time of administration of the first dose of CSL112 relative to contrast administration.

### Conclusion

The CSL112_2001 study of subjects with acute MI and moderate RI is supportive of renal safety with administration of 4 weekly infusions of CSL112 6 g compared with placebo in this population. The overall safety profile was favorable, and no new safety signals were identified that would warrant special monitoring for subjects with moderate RI compared to subjects with normal renal function or mild RI.

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety

### REFERENCES

1. Fox KAA, Dabbous O H, Goldberg R J, Pieper K S, Eagle K A, Van de Werf F, Avezum A, Goodman S G, Flather M D, Anderson F A, Granger C B. Prediction of risk of death and myocardial infarction in the six months after presentation with acute coronary syndrome: Prospective multinational observational study (grace). BMJ. 2006; 333:1091-1091
2. Stone G W, Maehara A, Lansky A J, de Bruyne B, Cristea E, Mintz G S, Mehran R, McPherson J, Farhat N, Marso S P, Parise H, Templin B, White R, Zhang Z, Serruys P W, Investigators P. A prospective natural-history study of coronary atherosclerosis. N Engl J Med. 2011; 364:226-235
3. Shotan A, Blondheim D S, Gottlieb S, Kazatsker M, Frimerman A, Shochat M, Garty M, Boyko V, Behar S, Meisel S R, Working Group on Intensive Cardiac C, Israel Heart S, Acute Coronary Syndrome Israeli Survey I. Comparison of outcome of recurrent versus first st-segment elevation myocardial infarction (from national israel surveys 1998 to 2006). The American journal of cardiology. 2011; 107:1730-1737
4. Anderson K M, Odell P M, Wilson P W, Kannel W B. Cardiovascular disease risk profiles. Am Heart J. 1991; 121:293-298
5. Wallis E J, Ramsay L E, Ul Haq I, Ghahramani P, Jackson P R, Rowland-Yeo K, Yeo W W. Coronary and cardiovascular risk estimation for primary prevention: Validation of a new sheffield table in the 1995 scottish health survey population. Bmj. 2000; 320:671-676
6. Anderson K M, Wilson P W, Odell P M, Kannel W B. An updated coronary risk profile. A statement for health professionals. Circulation. 1991; 83:356-362
7. Kannel W B, Dawber T R, Kagan A, Revotskie N, Stokes J, 3rd. Factors of risk in the development of coronary heart disease—six year follow-up experience. The framingham study. Ann Intern Med. 1961; 55:33-50
8. Assmann G, Cullen P, Schulte H. Simple scoring scheme for calculating the risk of acute coronary events based on the 10-year follow-up of the prospective cardiovascular munster (procam) study. Circulation. 2002; 105:310-315
9. Ferrario M, Chiodini P, Chambless L E, Cesana G, Vanuzzo D, Panico S, Sega R, Pilotto L, Palmieri L, Giampaoli S. Prediction of coronary events in a low incidence population. Assessing accuracy of the cuore cohort study prediction equation. International journal of epidemiology. 2005; 34:413-421
10. Conroy R M, Pyorala K, Fitzgerald A P, Sans S, Menotti A, De Backer G, De Bacquer D, Ducimetiere P, Jousilahti P, Keil U, Njolstad I, Oganov R G, Thomsen T, Tunstall-Pedoe H, Tverdal A, Wedel H, Whincup P, Wilhelmsen L, Graham I M. Estimation of ten-year risk of fatal cardiovascular disease in europe: The score project. Eur Heart J. 2003; 24:987-1003
11. Woodward M, Brindle P, Tunstall-Pedoe H. Adding social deprivation and family history to cardiovascular risk assessment: The assign score from the scottish heart health extended cohort (shhec). Heart. 2007; 93:172-176
12. Hippisley-Cox J, Coupland C, Vinogradova Y, Robson J, May M, Brindle P.

Derivation and validation of grisk, a new cardiovascular disease risk score for the united kingdom: Prospective open cohort study. Bmj. 2007; 335:136
13. Landray M J, Haynes R, Hopewell J C, Parish S, Aung T, Tomson J, Wallendszus K, Craig M, Jiang L, Collins R, Armitage J. Effects of extended-release niacin with laropiprant in high-risk patients. N Engl J Med. 2014; 371:203-212
14. Barter P J, Caulfield M, Eriksson M, Grundy S M, Kastelein J J P, Komajda M, Lopez-Sendon J, Mosca L, Tardif J-C, Waters D D, Shear C L, Revkin J H, Buhr K A, Fisher M R, Tall A R, Brewer B. Effects of torcetrapib in patients at high risk for coronary events. New England Journal of Medicine. 2007; 357:2109-2122
15. Boden W E, Probstfield J L, Anderson T, Chaitman B R, Desvignes-Nickens P, Koprowicz K, McBride R, Teo K, Weintraub W. Niacin in patients with low hdl cholesterol levels receiving intensive statin therapy. N Engl J Med. 2011; 365:2255-2267
16. Schwartz G G, Olsson A G, Abt M, Ballantyne C M, Barter P J, Brumm J, Chaitman B R, Holme I M, Kallend D, Leiter L A, Leitersdorf E, McMurray J J, Mundl H, Nicholls S J, Shah P K, Tardif J C, Wright R S, dal OI. Effects of dalcetrapib in patients with a recent acute coronary syndrome. N Engl J Med. 2012; 367:2089-2099
17. Tardif J C, Ballantyne C M, Barter P, Dasseux J L, Fayad Z A, Guertin M C, Kastelein J J, Keyserling C, Klepp H, Koenig W, L'Allier P L, Lesperance J, Luscher T F, Paolini J F, Tawakol A, Waters D D. Effects of the high-density lipoprotein mimetic agent cer-001 on coronary atherosclerosis in patients with acute coronary syndromes: A randomized trial. Eur Heart J. 2014; 35:3277-3286
18. Rohatgi A, Khera A, Berry J D, Givens E G, Ayers C R, Wedin K E, Neeland I J, Yuhanna I S, Rader D R, de Lemos J A, Shaul P W. Hdl cholesterol efflux capacity and incident cardiovascular events. N Engl J Med. 2014; 371:2383-2393
19. Saleheen D, Scott R, Javad S, Zhao W, Rodrigues A, Picataggi A, Lukmanova D, Mucksavage M L, Luben R, Billheimer J, Kastelein J J, Boekholdt S M, Khaw K T, Wareham N, Rader D J. Association of hdl cholesterol efflux capacity with incident coronary heart disease events: A prospective case-control study. The lancet. Diabetes & endocrinology. 2015; 3:507-513
20. Siddiqi H K, Kiss D, Rader D. Hdl-cholesterol and cardiovascular disease: Rethinking our approach. Current opinion in cardiology. 2015; 30:536-542
21. Ray KK, Ditmarsch M, Kallend D, Niesor E J, Suchankova G, Upmanyu R, Anzures-Cabrera J, Lehnert V, Pauly-Evers M, Holme I, Stasek J, van Hessen M W, Jones P, dal AI. The effect of cholesteryl ester transfer protein inhibition on lipids, lipoproteins, and markers of hdl function after an acute coronary syndrome: The dal-acute randomized trial. Eur Heart J. 2014; 35:1792-1800
22. Bounafaa A, Berrougui H, Ikhlef S, Essamadi A, Nasser B, Bennis A, Yamoul N, Ghalim N, Khalil A. Alteration of hdl functionality and pon1 activities in acute coronary syndrome patients. Clin Biochem. 2014; 47:318-325
23. Shao B, Tang C, Sinha A, Mayer P S, Davenport G D, Brot N, Oda M N, Zhao X Q, Heinecke J W. Humans with atherosclerosis have impaired abcal cholesterol efflux and enhanced high-density lipoprotein oxidation by myeloperoxidase. Circ Res. 2014; 114:1733-1742
24. Diditchenko S, Gille A, Pragst I, Stadler D, Waelchli M, Hamilton R, Leis A, Wright S D. Novel formulation of a reconstituted high-density lipoprotein (cs1112) dramatically enhances abcal-dependent cholesterol efflux. Arterioscler Thromb Vasc Biol. 2013; 33:2202-2211
25. Easton R, Gille A, D'Andrea D, Davis R, Wright S D, Shear C. A multiple ascending dose study of cs1112, an infused formulation of apoa-i. Journal of clinical pharmacology. 2014; 54:301-310
26. Gille A, Easton R, D'Andrea D, Wright S D, Shear C L. Cs1112 enhances biomarkers of reverse cholesterol transport after single and multiple infusions in healthy subjects. Arterioscler Thromb Vasc Biol. 2014; 34:2106-2114
27. Tricoci P, D'Andrea D M, Gurbel P A, Yao Z, Cuchel M, Winston B, Schott R, Weiss R, Blazing M A, Cannon L, Bailey A, Angiolillo D J, Gille A, Shear C L, Wright S D, Alexander J H. Infusion of reconstituted high-density lipoprotein, cs1112, in patients with atherosclerosis: Safety and pharmacokinetic results from a phase 2a randomized clinical trial. Journal of the American Heart Association. 2015; 4:e002171
28. Herzog E, Pragst I, Waelchli M, Gille A, Schenk S, Mueller-Cohrs J, Diditchenko S, Zanoni P, Cuchel M, Seubert A, Rader D J, Wright S D. Reconstituted high-density lipoprotein can elevate plasma alanine aminotransferase by transient depletion of hepatic cholesterol: Role of the phospholipid component. J Appl Toxicol. 2016; 36:1038-1047
29. Tardif J C, Gregoire J, L'Allier P L, Ibrahim R, Lesperance J, Heinonen™, Kouz S, Berry C, Basser R, Lavoie M A, Guertin M C, Rodes-Cabau J, Effect of r HDLoA-S, Efficacy I. Effects of reconstituted high-density lipoprotein infusions on coronary atherosclerosis: A randomized controlled trial. JAMA. 2007; 297:1675-1682
30. Thygesen K, Alpert J S, Jaffe A S, Simoons M L, Chaitman B R, White H D, Thygesen K, Alpert J S, White H D, Jaffe A S, Katus H A, Apple F S, Lindahl B, Morrow D A, Chaitman B R, Clemmensen P M, Johanson P, Hod H, Underwood R, Bax J J, Bonow R O, Pinto F, Gibbons R J, Fox KA, Atar D, Newby L K, Galvani M, Hamm C W, Uretsky B F, Steg P G, Wijns W, Bassand J-P, Menasche P, Ravkilde J, Ohman E M, Antman E M, Wallentin L C, Armstrong P W, Simoons M L, Januzzi J L, Nieminen M S, Gheorghiade M, Filippatos G, Luepker R V, Fortmann S P, Rosamond W D, Levy D, Wood D, Smith S C, Hu D, Lopez-Sendon J-L, Robertson R M, Weaver D, Tendera M, Bove A A, Parkhomenko A N, Vasilieva E J, Mendis S. Third universal definition of myocardial infarction. Journal of the American College of Cardiology. 2012; 60:1581-1598
31. Gibson C M, Korjian S, Tricoci P, Daaboul Y, Alexander J H, Steg P G, Lincoff A M, Kastelein J J P, Mehran R, D'Andrea D, Merkely B, Zarebinski M, Ophius T O, Harrington R A. Rationale and design of apo-i event reduction in ischemic syndromes i (aegis-i): A phase 2b, randomized, placebo-controlled, dose-ranging trial to investigate the safety and tolerability of cs1112, a reconstituted, infusible, human apoa-i, after acute myocardial infarction. American Heart Journal. 2016; 180:22-28
32. Nicholls S J. Accelerate: Evacetrapib does not reduce major cv events in high-risk patients. 2016, http://www.thecardiologyadvisor.com/acc-meeting-highlights/evacetrapib-did-not-reduce-cv-events-in-high-risk-patients/article/487228
33. Chapman M J, Le Goff W, Guerin M, Kontush A. Cholesteryl ester transfer protein: At the heart of the action of lipid-modulating therapy with statins, fibrates, niacin, and cholesteryl ester transfer protein inhibitors. Eur Heart J. 2010; 31:149-164
34. Investigators TA-H. Niacin in patients with low hdl cholesterol levels receiving intensive statin therapy. New England Journal of Medicine. 2011; 365:2255-2267
35. Bays H E, Shah A, Lin J, Sisk C M, Dong Q, Maccubbin D. Consistency of extended-release niacin/laropiprant effects on lp(a), apob, non-hdl-c, apo al, and apob/apoal ratio across patient subgroups. American journal of cardiovascular drugs:drugs, devices, and other interventions. 2012; 12:197-206
36. Maccubbin D, Bays H E, Olsson A G, Elinoff V, Elis A, Mitchel Y, Sirah W, Betteridge A, Reyes R, Yu Q, Kuznetsova O, Sisk C M, Pasternak R C, Paolini J F. Lipid-modifying efficacy and tolerability of extended-release niacin/laropiprant in patients with primary hypercholesterolaemia or mixed dyslipidaemia. International journal of clinical practice. 2008; 62:1959-1970
37. Fayad Z A, Mani V, Woodward M, Kallend D, Abt M, Burgess T, Fuster V, Ballantyne C M, Stein E A, Tardif J C, Rudd J H, Farkouh M E, Tawakol A. Safety and efficacy of dalcetrapib on atherosclerotic disease using novel non-invasive multimodality imaging (dal-plaque): A randomised clinical trial. Lancet. 2011; 378:1547-1559
38. Nicholls S J, Ruotolo G, Brewer H B, Kane J P, Wang M D, Krueger K A, Adelman S J, Nissen S E, Rader D J. Cholesterol efflux capacity and pre-beta-1 hdl concentrations are increased in dyslipidemic patients treated with evacetrapib. Journal of the American College of Cardiology. 2015; 66:2201-2210
39. Didichenko S A, Navdaev A V, Cukier A M, Gille A, Schuetz P, Spycher M O, Therond P, Chapman M J, Kontush A, Wright S D. Enhanced hdl functionality in small hdl species produced upon remodeling of hdl by reconstituted hdl, csl112: Effects on cholesterol efflux, anti-inflammatory and antioxidative activity. Circ Res. 2016; 119:751-763
40. Anderson W A D, Bethea W R. Renal lesions following administration of hypertonic solutions of sucrose. Report of six Cases. JAMA. 1940; 114(20):1983-7.
41. Bhandari S, Jain P. Management of acute coronary syndrome in chronic kidney disease. J Assoc Physicians India. 2012; 60:48-51.
42. Epstein J S, Zoon K C. Important drug warning: Immune globulin intravenous (human) (IGIV) products. Neonatal Netw. 2000; 19(2):60-2.
43. Fox KAA, Dabbous O H, Goldberg R J et al. Prediction of risk of death and myocardial infarction in the six months after presentation with acute coronary syndrome: prospective multinational observational study (GRACE). BMJ. 2006; 333: 1091-4.
44. Fox C S, Muntner P, Chen A Y, et al. Use of evidence-based therapies in short-term outcomes of ST-segment elevation myocardial infarction and non-ST-segment elevation myocardial infarction in patients with chronic kidney disease: A report from the National Cardiovascular Data Acute Coronary Treatment and Intervention Outcomes Network Registry. Circulation. 2010; 121:357-65.
45. Gibson C M, Dumaine R L, Gelfand E V, et al. Associated of glomerular filtration rate on presentation with subsequent mortality in non-ST-segment elevation acute coronary syndrome; observations in 13307 patients in five TIMI trials. Eur Heart J 2004; 25:1998-2005
46. Guidance for Industry: Drug-Induced Liver Injury: Premarketing Clinical Evaluation. July 2009 http://www.fda.gov/downloads/Drugs/ . . . /Guidances/UCM174090.pdf Kidney Disease: Improving Global Outcomes (KDIGO). Acute Kidney Injury Work Group. KDIGO clinical practice guidelines for acute kidney injury. Kidney Int Suppl 2012; 2:1.
47. Levey A S, Stevens L A, Schmid C H, et al. A new equation to estimate glomerular filtration rate. Ann Intern Med. 2009; 150(9):604-12.
48. Mehran R, Rao S V, Bhatt D L, et al. Standardized bleeding definitions for cardiovascular clinical trials. A consensus report from the Bleeding Academic Research Consortium. Circulation. 2011; 123:2736-47.
49. Nabais S, Rocha S, Joao C, et al. Prognostic impact of moderate renal dysfunction in acute coronary syndrome. Rev Port Cardiol. 2008; 27(3):303-12.
50. Pierce L R, Jain N. Risks associated with the use of intravenous immunoglobulin. Transfus Med Rev 2003; 17 (4): 241-51.
51. Stevens L A, Schmid C H, Greene T, et al. Comparative performance of the CKD epidemiology collaboration (CKD-EPI) and the modification of diet in renal disease (MDRD) study equations for estimating GFR levels above 60 mL/minute/1.73 m². American Journal of Kidney Diseases. 2010; 56(3):486-95.

TABLE 1
Baseline Characteristics

| | CSL112 2 g | | CSL112 6 g | | Placebo | | 3 way |
|---|---|---|---|---|---|---|---|
| Characteristic | (N = 419) | | (N = 421) | | (N = 418) | | p-value |
| Age, y - mean ± SD | 57.7 ± 10.1 | | 592 ± 9.9 | | 58.1 ± 10.6 | | 0.08 |
| Male gender - no. (%) | 337 | (80.4%) | 323 | (76.7%) | 341 | (81.6%) | 0.19 |
| Race - no. (%) | | | | | | | 0.57 |
| White | 404 | (96.7%) | 406 | (96.7%) | 409 | (97.9%) | |
| Black | 9 | (22%) | 5 | (1.2%) | 4 | (1.0%) | |
| Asian | 1 | (0.2%) | 4 | (1.0%) | 1 | (0.2%) | |
| Other | 4 | (1.0%) | 5 | (1.2%) | 4 | (1.0%) | |
| BMI, kg/m² - mean ± SD | 29.2 ± 6.3 | | 28.5 ± 5.0 | | 28.6 ± 5.2 | | 0.15 |
| eGFR, ml/min - mean ± SD | 86.1 ± 16.1 | | 86.6 ± 14.9 | | 87.4 ± 15.7 | | 0.49 |
| Renal function - no. (%) | | | | | | | 0.70 |
| Normal renal function | 194 | (46.4%) | 183 | (43.5%) | 188 | (45.0%) | |
| Mild renal impairment | 200 | (47.9%) | 219 | (52.0%) | 212 | (50.7%) | |
| Moderate/Severe renal impairment | 24 | (5.7%) | 19 | (4.5%) | 18 | (4.3%) | |
| Index Event - no. (%) | | | | | | | 0.20 |
| STEMI | 250 | (59.7%) | 274 | (65.1%) | 251 | (60.1%) | |
| NSTEMI | 169 | (40.3%) | 147 | (34.9%) | 167 | (40.0%) | |
| Index Interventional Procedure - no. (%) | | | | | | | 0.55 |
| PCI | 386 | (92.1%) | 397 | (94.3%) | 390 | (93.3%) | |
| CABG | 2 | (0.5%) | 0 | (0.0%) | 1 | (0.2%) | |
| Medical Therapy | 31 | (7.4%) | 24 | (5.7%) | 27 | (6.5%) | |
| Medical History - no. (%) | | | | | | | |
| Prior MI | 65 | (15.5%) | 58 | (13.8%) | 71 | (17.0%) | 0.44 |
| Stable angina | 65 | (15.5%) | 63 | (15.0%) | 58 | (13.9%) | 0.79 |
| Congestive heart failure | 24 | (5.7%) | 11 | (2.6%) | 18 | (4.3%) | 0.08 |
| Peripheral artery disease | 15 | (3.6%) | 14 | (3.3%) | 25 | (6.0%) | 0.11 |
| Cerebrovascular disease | 20 | (4.8%) | 21 | (5.0%) | 17 | (4.1%) | 0.80 |
| Hypertension | 269 | (64.2%) | 257 | (61.1%) | 240 | (57.4%) | 0.13 |
| Dyslipidemia | 222 | (53.0%) | 220 | (52.3%) | 222 | (53.1%) | 0.96 |
| Diabetes mellitus requiring treatment | 104 | (24.8%) | 81 | (19.2%) | 95 | (22.7%) | 0.15 |
| Smoking/tobacco use | 299 | (71.4%) | 292 | (69.4%) | 312 | (74.6%) | 0.23 |
| Timing of First Infusion from Angiography - no. | | | | | | | |
| (%) | | | | | | | |
| 12 h to <24 h | 9 | (22%) | 6 | (1.5%) | 9 | (2.2%) | 0.35 |
| 24 h to <48 h | 55 | (13.5%) | 76 | (18.5%) | 66 | (16.2%) | |
| ≥48 h | 344 | (84.3%) | 329 | (80.1%) | 332 | (81.6%) | |
| Timing of First Infusion from first medical contact, | 103 | (72.5-133.3) | 95.5 | (65.3-133.5) | 98.5 | (70.3-135.5) | 0.20 |
| hrs - median (IQR) | | | | | | | |
| Concomitant Medications* - no. (%) | | | | | | | |
| Statins | 391 | (94.2%) | 375 | (90.1%) | 387 | (93.7%) | 0.05 |
| High intensity or dose | 144 | (34.7%) | 132 | (31.7%) | 138 | (33.4%) | 0.66 |
| Low intensity or dose | 247 | (59.5%) | 243 | (58.4%) | 249 | (60.3%) | 0.86 |
| Other lipid lowering agents^{†} | 14 | (3.4%) | 11 | (2.6%) | 13 | (3.2%) | 0.82 |
| ACE inhibitor or ARB | 323 | (77.8%) | 325 | (78.1%) | 322 | (78.0%) | 0.99 |
| Beta blockers | 333 | (80.2%) | 319 | (76.7%) | 321 | (77.7%) | 0.44 |
| Aspirin | 406 | (97.8%) | 394 | (94.7%) | 400 | (96.9%) | 0.05 |
| Antiplatelet agents | 385 | (92.8%) | 395 | (95.0%) | 392 | (94.9%) | 0.31 |
| Anticoagulants | 34 | (8.2%) | 37 | (8.9%) | 42 | (10.2%) | 0.60 |

Baseline characteristics were calculated for patients at randomization.
For categorical variables a chi square test was used to calculate a p value, an ANOVA test for parametric continuous variables and a Kruskal-Wallis test was used.
eGFR is calculated using the Chronic Kidney Disease Epidemiology Collaboration Equation (2009). eGFR values summarized are the values derived from central laboratory serum creatinine values at screening. Where a central laboratory value is not available, local laboratory data are used.
For timing of first infusion from randomization, multiple pairwise comparisons were run: (6 g v. placebo = 0.002) and (2 g v. placebo = 0.1059) and (6 g v. 2 g = 0.3462).
^{†}Ezetimibe or PCSK9 Inhibitors
ACE denotes angiotensin converting hormone,
ARB angiotensin receptor blocker,
BMI body mass index,
CABG coronary artery bypass graft,
eGFR estimated glomerular filtration rate,
MI myocardial infarction,
NSTEMI non-ST-segment elevation myocardial infarction,
PCI percutaneous coronary intervention,
SD standard deviation, and
STEMI ST-segment elevation myocardial infarction.

TABLE 2
Co-primary Safety Endpoints

| | | Difference in | | | |
|---|---|---|---|---|---|
| Co-Primary | | rates | | Upper | |
| Safety | | (CSL11- | | Bound of | |
| Endpoint | n (%) | placebo) | 95% CI^{a} | 95% CI^{b} | p-value^{c} |
| Hepatic | | | | ≤14% | |
| CSL112 2 g | 4 (1.0%) | 1.0 | (−0.1, 2.5) | Yes | 0.12 |
| (N = 415) | | | | | |
| CSL112 6 g | 2 (0.5%) | 0.5 | (−0.5, 1.7) | Yes | 0.50 |
| (N = 416) | | | | | |
| Placebo | 0 (0.0%) | | | | |
| (N = 413) | | | | | |
| Renal | | | | <5% | |
| CSL112 2 g | 0 (0.0%) | −0.2 | (−1.4, 0.7) | Yes | 0.50 |
| (N = 415) | | | | | |
| CSL112 6 g | 3 (0.7%) | 0.5 | (−0.7, 1.9) | Yes | 0.62 |
| (N = 416) | | | | | |
| Placebo | 1 (0.2%) | | | | |
| (N = 413) | | | | | |

CI = Confidence Interval.
^{a}95% confidence intervals of the difference in the subject incidence rates are calculated using the Newcombe-Wilson score method.
^{b}Yes indicates non-inferiority criterion is met.
^{c}P values were calculated using Fisher's exact test.
The upper bound of the two-sided 95% confidence interval was specified for testing the co-primary endpoints, comparing with the specified thresholds for hepatic and renal endpoints for the non-inferiority assessment. This gives a one-sided 2.5% Type I error for each of the hepatic and renal endpoints and was based on an application of the Bonferroni method to control the overall Type I error at 5%.
Percentages are based on the number of subjects with data
A hepatic endpoint of interest is defined as any subject recording one of the two following results: ALT >3x ULN, Total bilirubin >2x ULN, confirmed by a consecutive repeat test after at least 24 hours but within 1 week of the original test.
A renal event is defined as a serum creatinine increase of ≥1.5X the baseline value, confirmed by a repeat test after at least 24 hours but within 1 week, or the need for renal replacement therapy.

TABLE 3
MACE Endpoints in the ITT population

| | 2 g | | 6 g | | Placebo | | HR | | p -value | HR | p-value |
|---|---|---|---|---|---|---|---|---|---|---|---|
| MACE Endpoint | (419) | | (421) | | (418) | | (2 g v. Placebo) | | (2 g v P) | (6g v. Placebo) | (6 g v P) |
| Composite 2° Endpoint | 27 | (6.4%) | 24 | (5.7%) | 23 | (5.5%) | 1.18 | (0.67, 2.05) | 0.5733 | 1.02 (0.57, 1.80) | 0.9717 |
| Composite 1 | 16 | (3.8%) | 20 | (4.8%) | 17 | (4.1%) | 0.93 | (0.47, 1.84) | 0.8391 | 1.15 (0.60, 2.20) | 0.6664 |
| Composite 2 | 16 | (3.8%) | 20 | (4.8%) | 17 | (4.1%) | 0.93 | (0.47, 1.85) | 0.8393 | 1.15 (0.60, 2.20) | 0.6660 |
| Composite 3 | 18 | (4.3%) | 20 | (4.8%) | 18 | (4.3%) | 0.99 | (0.51, 1.90) | 0.9705 | 1.09 (0.57, 2.05) | 0.7992 |
| Composite 4 | 34 | (8.1%) | 29 | (6.9%) | 31 | (7.4%) | 1.10 | (0.67, 1.78) | 0.7107 | 0.91 (0.55, 1.51) | 0.7008 |
| CV death | 2 | (0.5%) | 4 | (1.0%) | 0 | (0.0%) | — | | 0.3146 | — | 0.0477 |
| Non-fatal MI | 14 | (3.3%) | 13 | (3.1%) | 14 | (3.3%) | 0.99 | (0.47, 2.09) | 0.9828 | 0.91 (0.43, 1.93) | 0.7944 |
| Ischemic stroke | 0 | (0.0%) | 3 | (0.7%) | 3 | (0.7%) | — | | 0.1297 | 0.99 (0.20, 4.91) | 0.9918 |
| Hosp, for unstable angina | 13 | (3.1%) | 6 | (1.4%) | 7 | (1.7%) | 1.87 | (0.75, 4.69) | 0.1460 | 0.84 (0.28, 2.51) | 0.7766 |
| All-cause mortality | 5 | (1.2%) | 4 | (1.0%) | 1 | (0.2%) | 4.95 | (0.58, 42.37) | 0.1253 | 3.94 (0.44, 35.21) | 0.2526 |
| Non-CV death | 3 | (0.7%) | 0 | (0.0%) | 1 | (0.2%) | 2.92 | (0.30, 28.09) | 0.2341 | — | 0.5319 |
| Hemorrhagic stroke | 0 | (0.0%) | 1 | (0.2%) | 0 | (0.0%) | — | | 0.9914 | — | 0.2217 |
| Stroke - indeterminate | 0 | (0.0%) | 0 | (0.0%) | 0 | (0.0%) | — | | — | — | — |
| Any strokes | 0 | (0.0%) | 4 | (1.0%) | 3 | (0.7%) | — | | 0.1597 | 1.32 (0.30, 5.90) | 0.6515 |
| Heart failure | 5 | (1.2%) | 4 | (1.0%) | 1 | (0.2%) | 5.02 | (0.59, 43.01) | 0.1205 | 3.96 (0.44, 35.41) | 0.2525 |
| Coronary revascularization | 26 | (6.2%) | 17 | (4.0%) | 25 | (6.0%) | 1.05 | (0.60, 1.81) | 0.8669 | 0.66 (0.36, 1.22) | 0.1934 |

All numbers based upon a time-to-first MACE analysis in the ITT population
Percentages are based on the number of subjects with data.
All events were adjudicated by the CEC.
The hazard ratio is based on a proportional hazards model with factors for treatment group and renal function.
A hazard ratio <1 favors CSL112.
A stratified log-rank p-value < 0.05 indicates that the time-to-first-MACE in the CSL112 arm is significantly different when compared with placebo.
MACE Composite Secondary Endpoint consists of CV death, non-fatal MI, ischemic stroke and hospitalization for unstable angina.
Exploratory MACE Composite Endpoint 1 consists of CV death, non-fatal MI and ischemic stroke.
Exploratory MACE Composite Endpoint 2 consists of CV death, non-fatal MI and any strokes.
Exploratory MACE Composite Endpoint 3 consists of non-fatal MI, all-cause mortality and any strokes.
Exploratory MACE Composite Endpoint 4 consists of hospitalization for unstable angina, all-cause mortality, any strokes, heart failure and coronary revascularization.

TABLE 4
BARC Evaluation Grades for Worst Bleeding Events
Safety Population

| | CSL112 2 g | CSL112 6 g | Placebo |
|---|---|---|---|
| Bleeding Events | (415) | (416) | (413) |
| Type 0 | 377 (90.8%) | 378 (90.9%) | 362 (87.7%) |
| Type 1 | 19 (4.6%) | 17 (4.1%) | 30 (7.3%) |
| Type 2 | 16 (3.9%) | 17 (4.1%) | 15 (3.6%) |
| Type 3 | 2 (0.5%) | 3 (0.7%) | 6 (1.5%) |
| Type 3a | 0 | 0 | 2 |
| Type 3b | 2 | 3 | 3 |
| Type 3c | 0 | 0 | 1 |
| Type 4 | 0 (0.0%) | 0 (0.0%) | 0 (0.0%) |
| Type 5 | 1 (0.2%) | 1 (0.2%) | 0 (0.0%) |
| Type 5a | 0 | 0 | 0 |
| Type 5b | 1 | 1 | 0 |

Type 0 includes subjects who had no bleeding events to adjudicate
If a patient had greater than one bleed, the most severe bleed was counted
Bleeding events were counted from randomization

TABLE 5
Baseline Lipid and Cardiovascular Biomarkers

| | | | | |
|---|---|---|---|---|
| Biomarker | CSL112 2 g | CSL112 6 g | Placebo | p-value |

Plasma Biomarkers

| | | | | |
|---|---|---|---|---|
| ApoA-I | 124.6 ± 24.6 | 127.7 ± 25.2 | 126.1 ± 24.7 | 0.2155 |
| mg/dL - Mean ± SD | | | | |
| Phosphatidylcholine | 185.9 ± 36.6 | 190.1 ± 39.2 | 187.3 ± 37.7 | 0.2835 |
| mg/dL - Mean ± SD | | | | |

Lipid Biomarkers

| | | | | |
|---|---|---|---|---|
| Apolipoprotein B | 90.8 ± 24.3 | 92.8 ± 25.3 | 91.9 ± 25.4 | 0.5308 |
| mg/dL - Mean ± SD | | | | |
| Total Cholesterol | 164.7 ± 39.3 | 169.3 ± 41.0 | 166.5 ± 41.6 | 0.2735 |
| mg/dL - Mean ± SD | | | | |
| HDL Cholesterol | 40.2 ± 11.0 | 41.6 ± 10.7 | 40.8 ± 11.0 | 0.1606 |
| mg/dL - Mean ± SD | | | | |
| Non-HDL Cholesterol | 124.2 ± 38.9 | 127.6 ± 40.4 | 125.8 ± 40.9 | 0.4780 |
| mg/dL - Mean ± SD | | | | |
| LDL Cholesterol | 92.1 ± 35.0 | 94.7 ± 34.9 | 92.1 ± 34.4 | 0.4966 |
| mg/dL - Mean ± SD | | | | |
| Triglycerides | 168.8 ± 99.5 | 168.0 ± 91.3 | 170.2 ± 95.1 | 0.9450 |
| mg/dL - Mean ± SD | | | | |

Cholesterol Efflux Capacity

| | | | | |
|---|---|---|---|---|
| Total Efflux | 8.4 ± 2.3 | 8.8 ± 2.9 | 8.8 ± 2.7 | 0.1299 |
| %/4 h - Mean ± SD | | | | |
| ABCA1 Efflux | 2.6 ± 1.7 | 2.6 ± 1.9 | 2.8 ± 1.9 | 0.2097 |
| %/4 h - Mean ± SD | | | | |
| Non-ABCA1 Efflux | 5.9 ± 1.4 | 6.2 ± 1.6 | 6.0 ± 1.5 | 0.0305 |
| %/4 h - Mean ± SD | | | | |
| Total EC/ApoA-I | 0.068 ± 0.017 | 0.069 ± 0.020 | 0.070 ± 0.021 | 0.3304 |
| Ratio | | | | |
| %/4 h/mg/dL - Mean + SD | | | | |
| ABCA1 EC/ApoA-I | 0.021 ± 0.013 | 0.021 ± 0.014 | 0.022 ± 0.015 | 0.1401 |
| Ratio | | | | |
| %/4 h/mg/dL - Mean + SD | | | | |

Cardiovascular Biomarkers

| | | | | |
|---|---|---|---|---|
| Troponin I | 53.9 ± 400.2 | 48.2 ± 187.9 | 67.8 ± 361.6 | 0.8618 |
| ng/mL - Mean ± SD | | | | |
| Fibrinogen | 481.7 ± 122.0 | 482.2 ± 125.0 | 476.3 ± 125.6 | 0.7588 |
| mg/dL - Mean ± SD | | | | |
| hsCRP | 18.9 ± 28.9 | 18.7 ± 23.7 | 18.4 ± 27.5 | 0.9677 |
| mg/L - Mean ± SD | | | | |
| IL-6 | 9.2 ± 45.8 | 8.3 ± 21.5 | 7.4 ± 9.8 | 0.6754 |
| pg/mL - Mean ± SD | | | | |

All analyses were based off patients with available data.
CEC = Cholesterol Efflux Capacity,
CI = C onfidence Interval
ABCA1 denotes ATP-binding cassette A1,
HDL high density lipoprotein,
hsCRP high sensitivity c-reactive protein,
IL-6 interlukin-6,
LDL low density lipoprotein,
NT-proBNP N-terminal prohormone of brain natriuretic peptide, and
SD standard deviation.
^{a}Treatment comparison based on ANOVA with terms for treatment group.

TABLE 6
Cholesterol Efflux, HDL-Cholesterol and apoA-I values
immediately after infusion of CSL112

| | | |
|---|---|---|
| Parameter | Arithmetic Mean ± SD | Fold Elevation |

Total Cholesterol Efflux Capacity (%/4 h)

| | | |
|---|---|---|
| CSL112 2 g (N = 394) | 15.8 ± 3.8 | 1.87^{#} |
| CSL112 6 g (N = 404) | 20.8 ± 3.8 | 2.45^{#} |
| Placebo (N = 403) | 8.3 ± 2.7 | 0.94^{#} |

ABCA1-Dependent Cholesterol Efflux Capacity (%/4 h)

| | | |
|---|---|---|
| CSL112 2 g (N = 394) | 7.9 ± 2.6 | 3.67^{#} |
| CSL112 6 g (N = 404) | 8.9 ± 2.4 | 4.30^{#} |
| Placebo (N = 403) | 2.4 ± 1.8 | 0.82^{#} |

ApoA-I (mg/dl)

| | | |
|---|---|---|
| CSL112 2 g (N = 402) | 161 ± 33.4 | 1.29^{#} |
| CSL112 6 g (N = 406) | 263 ± 58.2 | 2.06^{#} |
| Placebo (N = 405) | 121 ± 25.7 | 0.96^{#} |

HDL-Cholesterol mg/dL

| | | |
|---|---|---|
| CSL112 2 g (N = 404) | 43.9 ± 11.8 | 1.09^{#} |
| CSL112 6 g (N = 407 | 52.5 ± 12.1 | 1.27^{#} |
| Placebo (N = 405) | 39.3 ± 10.9 | 0.97^{#} |

All analyses were based on patients with available data.
^{#}Fold elevation compared with baseline, calculated as a geometric mean of the individual patient ratios

TABLE 7
Sensitivity Analyses of Co-Primary Safety Endpoints

| | | Difference in | | | |
|---|---|---|---|---|---|
| Co-Primary | | rates | | Upper | |
| Safety | | (CSL11- | | Bound of | |
| Endpoint | n (%) | placebo) | 95% CI^{a} | 95% CI^{b} | p-value^{c} |
| Hepatic - No | | | | ≤4% | |
| Confirmatory Result | | | | | |
| CSL112 2 gB(N = 415) | 9 (2.2%) | −0.5 | (−2.8, 1.7) | Yes | 0.64 |
| CSL112 6 gB(N = 416) | 5 (1.2%) | −1.5 | (−3.6, 0.5) | Yes | 0.13 |
| PlaceboB(N = 413) | 11 (2.7%) | | | | |
| Renal - No | | | | <5% | |
| Confirmatory Result | | | | | |
| CSL112 2 g (N = 415) | 4 (1.0%) | −0.5 | (−2.3, 1.2) | Yes | 0.55 |
| CSL112 6 g (N = 416) | 7 (1.7%) | 0.2 | (−1.7, 2.1) | Yes | 0.79 |
| Placebo (N = 413) | 6 (1.5%) | | | | |

CI = Confidence Interval.
^{a}95% confidence intervals of the difference in the subject incidence rates are calculated using the Newcombe-Wilson score method.
^{b}Yes indicates non-inferiority criterion is met.
^{c}*P values were calculated using Chi-Square test or Fisher's exact test when expected cell counts were <5.
* Percentages are based on the number of subjects with data.
* For this sensitivity analysis, a hepatic endpoint of interest is defined as any subject recording one of the two following results: ALT >3xULN, Total bilirubin >2xULN, without confirmation using a consecutive repeat test after at least 24 hours but within 1 week of original test.
* For this sensitivity analysis, a renal event is defined as a serum creatinine increase of ≥1.5X the baseline value or the need for renal replacement therapy, without confirmation using a consecutive repeat test after at least 24 hours but within 1 week of original test.

TABLE 8
Post-hoc Sensitivity Analysis of Co-Primary Safety Endpoints
with Bonferroni Adjustment for Multiple Treatment Comparisons

| | | Difference in | | | |
|---|---|---|---|---|---|
| Co-Primary | | rates | | Upper | |
| Safety | | (CSL11- | | Bound of | |
| Endpoint | n (%) | placebo) | 97.5% CI^{a} | 97.5% CI^{b} | p-value^{c} |
| Hepatic | | | | ≤4% | |
| CSL112 2 g | 4 (1.0%) | 1.0 | (−0.4, 2.8) | Yes | 0.12 |
| (N = 415) | | | | | |
| CSL112 6 g | 2 (0.5%) | 0.5 | (−0.8, 2.0) | Yes | 0.50 |
| (N = 416) | | | | | |
| Placebo | 0 (0.0%) | | | | |
| (N = 413) | | | | | |
| Renal | | | | <5% | |
| CSL112 2 g | 0 (0.0%) | −0.2 | (−1.7, 1.0) | Yes | 0.50 |
| (N = 415) | | | | | |
| CSL112 6 g | 3 (0.7%) | 0.5 | (−1.0, 2.2) | Yes | 0.62 |
| (N = 416) | | | | | |
| Placebo | 1 (0.2%) | | | | |
| (N = 413) | | | | | |

CI = Confidence Interval.
^{a}The upper bound of the two-sided 95% confidence interval was specified for testing the co-primary endpoints, comparing with the specified thresholds for hepatic and renal endpoints for the non-inferiority assessment. This gives a one-sided 2.5% Type I error for each of the hepatic and renal endpoints and was based on an application of the Bonferroni method to control the overall Type I error at 5%. Multiplicity adjustment was not applied to the two pairwise treatment group comparisons within each co-primary endpoint. This table displays a more conservative assessment using a two-sided 97.5% confidence interval, which further applies a post-hoc Bonferroni adjustment to the treatment group comparisons to achieve an individual one-sided 1.25% Type I error for each of the treatment group comparisons.
^{b}Yes indicates non-inferiority criterion is met.
^{c}P values were calculated using Fisher's exact test.
Percentages are based on the number of subjects with data
A hepatic endpoint of interest is defined as any subject recording one of the two following results: ALT >3xULN, Total bilirubin >2xULN, confirmed by a consecutive repeat test after at least 24 hours but within 1 week of the original test.
A renal event is defined as a serum creatinine increase of ≥1.5X the baseline value, confirmed by a repeat test after at least 24 hours but within 1 week, or the need for renal replacement therapy.

TABLE 9
Cholesterol Efflux and apoA-I ratios immediately after infusion
of CSL112

| | | |
|---|---|---|
| | | Fold |
| Parameter | Arithmetic Mean ± SD | Elevation |

Total Cholesterol Efflux Capacity/ApoA-I Ratio (%/4 hr/mg/dL)

| | | |
|---|---|---|
| CSL112 2 g (N = 394) | 0.099 ± 0.023 | 1.44^{@} |
| CSL112 6 g (N = 404) | 0.082 ± 0.019 | 1.18^{@} |
| Placebo (N = 403) | 0.069 ± 0.019 | — |

ABCA1 Cholesterol Efflux Capacity/ApoA-I Ratio (%/4 hr/mg/dL)

| | | |
|---|---|---|
| CSL112 2 g (N = 394) | 0.050 ± 0.017 | 2.51^{@} |
| CSL112 6 g (N = 404) | 0.035 ± 0.013 | 1.78^{@} |
| Placebo (N = 403) | 0.020 ± 0.014 | — |

All analyses were based on patients with available data.
@Fold elevation compared with placebo, calculated as a ratio of the treatment arithmetic means

TABLE 10
Treatment Emergent Adverse Events, Frequency of Events Safety Population

| | CSL112 2 g | | CSL112 6 g | | Placebo | |
|---|---|---|---|---|---|---|
| Adverse Event (System Organ Class) | (696) | | (620) | | (639) | |
| Blood & Lymphatic | 11 | (1.6%) | 2 | (0.3%) | 5 | (0.8%) |
| Cardiac | 74 | (10.6%) | 61 | (9.8%) | 61 | (9.6%) |
| Congenital, Familial & Genetic | 0 | (0.0%) | 1 | (0.2%) | 1 | (0.2%) |
| Ear & Labyrinth | 2 | (0.3%) | 7 | (1.1%) | 6 | (0.9%) |
| Endocrine | 1 | (0.1%) | 1 | (0.2%) | 5 | (0.8%) |
| Eye | 3 | (0.4%) | 10 | (1.6%) | 6 | (0.9%) |
| Gastrointestinal | 61 | (8.8%) | 67 | (10.8%) | 68 | (10.6%) |
| General Disorders & Administration Site | 122 | (17.5%) | 92 | (14.8%) | 92 | (14.4%) |
| Conditions | | | | | | |
| Hepatobiliary | 7 | (1.0%) | 0 | (0.0%) | 6 | (0.9%) |
| Immune System | 2 | (0.3%) | 2 | (0.3%) | 0 | (0.0%) |
| Infections & Infestations | 61 | (8.8%) | 49 | (7.9%) | 42 | (6.6%) |
| Injury, Poisoning & Procedural Complication | 25 | (3.6%) | 30 | (4.8%) | 27 | (4.2%) |
| Investigations | 39 | (5.6%) | 54 | (8.7%) | 57 | (8.9%) |
| Metabolism & Nutrition | 24 | (3.5%) | 10 | (1.6%) | 21 | (3.3%) |
| Musculoskeletal & Connective Tissue | 46 | (6.6%) | 42 | (6.8%) | 33 | (5.2%) |
| Neoplasms Benign, Malignant & Unspecified | 0 | (0.0%) | 6 | (1.0%) | 7 | (1.1%) |
| (Incl. Cysts & Polyps) | | | | | | |
| Nervous System | 66 | (9.5%) | 52 | (8.4%) | 53 | (8.3%) |
| Product Issues | 2 | (0.3%) | 0 | (0.0%) | 0 | (0.0%) |
| Psychiatric | 14 | (2.0%) | 9 | (1.5%) | 10 | (1.6%) |
| Renal & Urinary | 16 | (2.3%) | 6 | (1.0%) | 15 | (2.4%) |
| Reproductive System & Breast | 5 | (0.7%) | 6 | (1.0%) | 4 | (0.6%) |
| Respiratory, Thoracic & Mediastinal | 63 | (9.1%) | 52 | (8.4%) | 58 | (9.1%) |
| Skin & Subcutaneous Tissue | 17 | (2.4) | 24 | (3.9%) | 17 | (2.7%) |
| Vascular | 35 | (5.0%) | 37 | (6.0%) | 45 | (7.0%) |
| Other | | | | | | |
| Adverse event related to study drug? | 44 | (6.3%) | 50 | (8.1%) | 34 | (5.3%) |
| Adverse event related to study procedure | 27 | (3.9%) | 43 | (6.9%) | 15 | (2.4%) |
| Adverse events leading to death | 3 | (0.4%) | 2 | (0.3%) | 1 | (0.2%) |
| Adverse events leading to permanent | 11 | (1.6%) | 8 | (1.3%) | 9 | (1.4%) |
| discontinuation of study drug | | | | | | |
| Severity of adverse events* | | | | | | |
| Grade 1 | 409 | (58.8%) | 374 | (60.3%) | 388 | (60.7%) |
| Grade 2 | 151 | (21.7%) | 165 | (26.6%) | 147 | (23.0%) |
| Grade 3 | 114 | (16.4%) | 76 | (12.3%) | 92 | (14.4%) |
| Grade 4 | 18 | (2.6%) | 3 | (0.5%) | 11 | (1.7%) |
| Grade 5 | 4 | (0.6%) | 2 | (0.3%) | 1 | (0.2%) |
| Serious adverse events | 109 | (15.7%) | 77 | (12.4%) | 78 | (12.2%) |
| Serious related adverse events | 1 | (0.8%) | 0 | (0.0%) | 2 | (5.9%) |

The N's represent the total number of adverse events in each treatment group

TABLE 11
Treatment Emergent Adverse Events, Percentage of Patients Safety Population

| | CSL112 2 g | | CSL112 6 g | | Placebo | |
|---|---|---|---|---|---|---|
| Adverse Event (System Organ Class) | (N = 415) | | (N = 416) | | (N = 413) | |
| Blood & Lymphatic | 10 | (2.4%) | 2 | (0.5%) | 4 | (1.0%) |
| Cardiac | 51 | (12.3%) | 48 | (11.5%) | 40 | (9.7%) |
| Congenital, Familial & Genetic | 0 | (0.0%) | 1 | (0.2%) | 1 | (0.2%) |
| Ear & Labyrinth | 2 | (0.5%) | 7 | (1.7%) | 5 | (1.2%) |
| Endocrine | 1 | (0.2%) | 1 | (0.2%) | 4 | (1.0%) |
| Eye | 3 | (0.7%) | 6 | (1.4%) | 4 | (1.0%) |
| Gastrointestinal | 42 | (10.1%) | 42 | (10.1%) | 46 | (11.1%) |
| General Disorders & Administration Site | 84 | (20.2%) | 62 | (14.9%) | 62 | (15.0%) |
| Conditions | | | | | | |
| Hepatobiliary | 6 | (1.5%) | 0 | (0.0%) | 5 | (1.2%) |
| Immune System | 2 | (0.5%) | 2 | (0.5%) | 0 | (0.0%) |
| Infections & Infestations | 47 | (11.3%) | 39 | (9.4%) | 38 | (9.2%) |
| Injury, Poisoning & Procedural Complication | 18 | (4.3%) | 17 | (4.1%) | 24 | (5.8%) |
| Investigations | 31 | (7.5%) | 37 | (8.9%) | 41 | (9.9%) |
| Metabolism & Nutrition | 19 | (4.6%) | 10 | (2.4%) | 18 | (4.4%) |
| Musculoskeletal & Connective Tissue | 35 | (8.4%) | 35 | (8.4%) | 24 | (5.8%) |
| Neoplasms Benign, Malignant & Unspecified | 0 | (0.0%) | 6 | (1.4%) | 7 | (17%) |
| (Incl. Cysts & Polyps) | | | | | | |
| Nervous System | 47 | (11.3%) | 45 | (10.8%) | 30 | (7.3%) |
| Product Issues | 2 | (0.5%) | 0 | (0.0%) | 0 | (0.0%) |
| Psychiatric | 13 | (3.1%) | 8 | (1.9%) | 9 | (2.2%) |
| Renal & Urinary | 16 | (3.9%) | 6 | (1.4%) | 14 | (3.4%) |
| Reproductive System & Breast | 4 | (1.0%) | 6 | (1.4%) | 4 | (1.0%) |
| Respiratory, Thoracic & Mediastinal | 43 | (10.4%) | 42 | (10.1%) | 42 | (10.2%) |
| Skin & Subcutaneous Tissue | 14 | (3.4%) | 22 | (5.3%) | 17 | (4.1%) |
| Vascular | 30 | (7.2%) | 32 | (7.7%) | 38 | (9.2%) |
| Other | | | | | | |
| Study-drug Related adverse events | 33 | (8.0%) | 33 | (7.9%) | 26 | (6.3%) |
| Adverse events leading to death | 3 | (0.7%) | 2 | (0.5%) | 1 | (0.2%) |
| Adverse events leading to permanent | 11 | (2.7%) | 8 | (1.9%) | 9 | (2.2%) |
| discontinuation of study drug | | | | | | |
| Severity of adverse events* | | | | | | |
| Grade 1 | 73 | (17.6%) | 91 | (21.9%) | 88 | (21.3%) |
| Grade 2 | 60 | (14.5%) | 69 | (16.6%) | 52 | (12.6%) |
| Grade 3 | 69 | (16.6%) | 50 | (12.0%) | 56 | (13.6%) |
| Grade 4 | 5 | (1.2%) | 2 | (0.5%) | 8 | (1.9%) |
| Grade 5 | 3 | (0.7%) | 2 | (0.5%) | 1 | (0.2%) |
| Serious adverse events | 66 | (15.9%) | 53 | (12.7%) | 54 | (13.1%) |
| Serious related adverse events | 1 | (0.2%) | 0 | (0.0%) | 2 | (0.5%) |

The N's represent the percentage of patients that experienced an adverse event by treatment group.
*If a patient experienced greater than one adverse event, the most severe was presented for severity of adverse event.

TABLE 12
Summary of Fatal Outcomes by Study Period

| Treatment Period | 2 g | 6 g | Placebo |
|---|---|---|---|
| Main Study (N = 10) | 5 | 4 | 1 |
| Active Treatment Period (SD 1-29) | 1 | 2 | 0 |
| Safety Follow-Up Period (SD 30-112) | 2 | 0 | 1 |
| MACE Follow-Up Period (SD 113-387) | 2 | 2 | 0 |
| Safey Lead In (N = 1) | 1 | 0 | 0 |
| Active Treatment Period (SD 1-29) | 0 | 0 | 0 |
| Safety Follow-Up Period (SD 30-90) | 1 | 0 | 0 |

TABLE 13
Sample size and power calculation of MACE endpoints required to
detect a ≥15% risk reduction at a two-sided significance level of 0.05

| | | No. per | Power with |
|---|---|---|---|
| | Placebo | group required | 420 subjects |
| MACE Endpoint | Event Rate | for 90% power | per group |
| Composite 2° Endpoint | 5.5% | 14,907 | 8.4% |
| Composite 1 | 4.1% | 20,271 | 7.5% |
| Composite 2 | 4.1% | 20,271 | 7.5% |
| Composite 3 | 4.3% | 19,291 | 7.7% |
| Composite 4 | 7.4% | 10,874 | 9.8% |
| CV death | 0.0% | — | — |
| Non-fatal MI | 3.3% | 25,379 | 7.0% |
| Ischemic stroke | 0.7% | 122,620 | 5.4% |
| Hosp. for unstable angina | 1.7% | 50,019 | 6.0% |
| All-cause mortality | 0.2% | 431,171 | 5.1% |
| Non-CV death | 0.2% | 431,171 | 5.1% |
| Hemorrhagic stroke | 0.0% | — | — |
| Stroke - indeterminate | 0.0% | — | — |
| Any stroke | 0.7% | 122,620 | 5.4% |
| Heart failure | 0.2% | 431,171 | 5.1% |
| Coronary revascularization | 6.0% | 13,598 | 8.8% |

Sample size and power were calculated based on the observed event rate in the placebo arm using the Pearson's chi-square test.
For this power calculation both the treatment and placebo arms were standardized to 420 patients.

TABLE 14
Baseline characteristics

| | NRF | Mod RI |
|---|---|---|
| | n = 16 | n = 16 |
| Age, years | 55 ± 7 | 69 ± 9 |
| Sex, n (%) male | 11 (68.8) | 11 (68.8) |
| Weight, kg | 78 ± 10.8 | 80.5 ± 16.6 |
| BMI, kg/m² | 26.23 ± 2.89 | 27.88 ± 4.64 |
| eGFR, mL/min/1.73 m² | 100.5 ± 6.0 | 49.1 ± 7.7 |

BMI, body mass index;
Shown are means ± standard deviation

TABLE 15
Baseline cholesterol efflux and lipoprotein parameters
by renal function group

| | NRF | Mod RI | p-value for |
|---|---|---|---|
| | n = 16 | n = 16 | comparison |
| ApoA-I, mg/dL | 141 ± 19.0 | 143 ± 21.3 | 0.8 |
| Total CEC, % efflux/4 h | 9.03 ± 1.75 | 11.50 ± 2.49 | 0.003 |
| ABCA1-independent CEC, % | 7.02 ± 1.29 | 7.85 ± 1.56 | 0.1 |
| efflux/4 h | | | |
| ABCA1-dependent CEC, | 2.01 ± 1.22 | 3.65 ± 1.68 | 0.004 |
| % efflux/4 h | | | |
| Pre-β1-HDL, μg/mL | 16.1 ± 3.2 | 22.8 ± 9.8 | 0.01 |
| Cholesterol, mg/dL | 191 ± 36 | 188 ± 34 | 0.8 |
| HDL-cholesterol, mg/dL | 52 ± 8 | 53 ± 12 | 0.7 |
| HDL-unesterified cholesterol, | 15 ± 3 | 15 ± 3 | 1.0 |
| mg/dL | | | |
| HDL-esterified cholesterol, | 37 ± 6 | 38 ± 9 | 0.7 |
| mg/dL | | | |
| Non-HDL-cholesterol, mg/dL | 140 ± 37 | 136 ± 31 | 0.7 |
| Apolipoprotein B, mg/dL | 91 ± 24 | 89 ± 18 | 0.8 |
| Triglycerides, mg/dL | 132 ± 57 | 141 ± 63 | 0.7 |
| C-reactive protein, mg/L | 1.7 ± 3.5 | 2.3 ± 3.9 | 0.6 |

Shown are means ± standard deviation

TABLE 16
Study Population Characteristics

| | CSL112 6 g | Placebo | Total |
|---|---|---|---|
| Characteristics | (N = 55) | (N = 28) | (N = 83) |
| Age (years) | | | |
| N | 55 | 28 | 83 |
| Mean (SD) | 70.6 (10.95) | 71.9 (10.12) | 71.1 (10.63) |
| Median | 73.0 | 74.0 | 73.0 |
| 1^{st} quartile, 3^{rd} quartile | 65.0, 79.0 | 69.0, 78.0 | 66.0, 78.0 |
| Min, Max | 36, 86 | 44, 89 | 36, 89 |
| Age Group (years), n (%) | | | |
| ≥18-<65 | 11 (20.0) | 4 (14.3) | 15 (18.1) |
| ≥65-<75 | 20 (36.4) | 11 (39.3) | 31 (37.3) |
| ≥75-<85 | 22 (40.0) | 12 (42.9) | 34 (41.0) |
| ≥85 | 2 (3.6) | 1 (3.6) | 3 (3.6) |
| Sex n (%) | | | |
| Male | 37 (67.3) | 18 (64.3) | 55 (66.3) |
| Female | 18 (32.7) | 10 (35.7) | 28 (33.7) |
| Ethnicity, n (%) | | | |
| Hispanic or Latino | 0 | 2 (7.1) | 2 (2.4) |
| Not Hispanic or Latino | 53 (96.4) | 26 (92.9) | 79 (95.2) |
| Unknown | 2 (3.6) | 0 | 2 (2.4) |
| Race n, (%) | | | |
| Asian | 1 (1.8) | 0 | 1 (1.2) |
| Black or African American | 2 (3.6) | 0 | 2 (2.4) |
| White | 52 (94.5) | 28 (100) | 80 (96.4) |
| Country, n (%) | | | |
| Germany | 12 (21.8) | 4 (14.3) | 16 (19.3) |
| Hungary | 20 (36.4) | 8 (28.6) | 28 (33.7) |
| Israel | 5 (9.1) | 5 (17.9) | 10 (12.0) |
| Netherlands | 8 (14.5) | 2 (7.1) | 10 (12.0) |
| United States | 10 (18.2) | 9 (32.1) | 19 (22.9) |
| BMI (kg/m²) | | | |
| N | 55 | 28 | 83 |
| Mean (SD) | 30.0 (5.30) | 28.5 (4.68) | 29.5 (5.12) |
| Median | 29.4 | 28.4 | 29.1 |
| 1^{st} quartile, 3^{rd} quartile | 26.5, 32.2 | 25.0, 31.1 | 25.9, 31.6 |
| Min, Max | 19.8, 46.1 | 21.3, 43.1 | 19.8, 46.1 |
| Renal function (Randomized)^{a}, n (%) | | | |
| eGFR 30-<45 mL/min/1.73 m² | 26 (47.3) | 13 (46.4) | 39 (47.0) |
| eGFR 45-<60 mL/min/1.73 m² | 29 (52.7) | 15 (53.6) | 44 (53.0) |
| Renal function (Central Laboratory)^{b}, n (%) | | | |
| eGFR <30 mL/min/1.73 m² | 3 (5.5) | 1 (3.6) | 4 (4.8) |
| eGFR 30-<45 mL/min/1.73 m² | 18 (32.7) | 15 (53.6) | 33 (39.8) |
| eGFR 45-<60 mL/min/1.73 m² | 26 (47.3) | 11 (39.3) | 37 (44.6) |
| eGFR ≥60 mL/min/1.73 m² | 4 (7.3) | 1 (3.6) | 5 (6.0) |
| eGFR (IRT) at Randomization^{c}, mL/min/1.73 m² | | | |
| N | 55 | 28 | 83 |
| Mean (SD) | 46.15 (7.165) | 46.41 (7.785) | 46.24 (7.334) |
| Median | 45.91 | 45.16 | 45.61 |
| 1^{st} quartile, 3^{rd} quartile | 40.71, 52.92 | 39.59, 53.36 | 40.33, 52.92 |
| Min, Max | 30.2, 57.8 | 33.5, 59.2 | 30.2, 59.2 |
| eGFR (Central) at Randomization^{d}, mL/min/1.73 m² | | | |
| N | 51 | 28 | 79 |
| Mean (SD) | 46.82 (9.697) | 45.40 (9.988) | 46.32 (9.761) |
| Median | 48.99 | 42.50 | 45.44 |
| 1^{st} quartile, 3^{rd} quartile | 38.53, 55.10 | 37.71, 53.91 | 38.10, 55.10 |
| Min, Max | 27.3, 64.4 | 29.8, 70.9 | 27.3, 70.9 |
| Diabetes requiring current treatment with | | | |
| any anti-diabetic medication^{e}, n (%) | | | |
| Yes | 23 (41.8) | 12 (42.9) | 35 (42.2) |
| No | 32 (58.2) | 16 (57.1) | 48 (57.8) |
| Type of Index MI, n (%) | | | |
| STEMI | 16 (29.1) | 6 (21.4) | 22 (26.5) |
| NSTEMI | 39 (70.9) | 22 (78.6) | 61 (73.5) |

BMI = body mass index,
eCRF = electronic case report form,
eGFR = estimated Glomerular Filtration Rate,
IRT = interactive response technology,
ITT = Intent-to-Treat,
Max = maximum,
MI = myocardial infarction,
Min = minimum,
NSTEMI = non ST-segment elevation myocardial infarction,
SD = standard Deviation,
STEMI = ST-segment elevation myocardial infarction
^{a}Stratum to which subject was assigned from the IRT system initial calculation of eGFR based on the subject's age, sex, race, and the serum creatinine value obtained at Visit 2 (Study Day 1).
^{b}Stratum to which the subject belonged based on the calculation of eGFR using the Chronic Kidney Disease-Epidemiology Collaboration equation and the central laboratory serum creatinine value obtained at Visit 2 (Study Day 1).
^{c}eGFR values as recorded within the IRT system
^{d}eGFR values summarized were calculated using the Chronic Kidney Disease-Epidemiology Collaboration equation using serum creatinine values derived from central laboratory at Visit 2 (Study Day 1).
^{e}Medical history of diabetes as recorded on the eCRF.
Note:
Percentages were based on the number of subjects randomized within each treatment group. Age was automatically calculated from the date of birth and date of informed consent. Baseline for non-laboratory data was defined as the most recent pre-infusion, non-missing value prior to or on the first study treatment dose date.

TABLE 17
Summary of Timings Up to First Infusion (ITT Population)

| | | Overall | | | STEMI | |
|---|---|---|---|---|---|---|
| | | CSL112 6 g | Placebo | Total | CSL112 6 g | Placebo |
| Timing Characteristics | Descriptive Statistic | (N = 55) | (N = 28) | (N = 83) | (N = 55) | (N = 28) |

Time between Index MI and Angiography (h)

| N | 54 | | 26 | | 80 | | 16 | | 5 | |
|---|---|---|---|---|---|---|---|---|---|---|
| Mean (SD) | 16.64 | (17.347) | 20.47 | (20.242) | 17.89 | (18.298) | 4.54 | (6.718) | 2.58 | (2.627) |

Time between Angiography and Randomization (h)

| N | 54 | | 26 | | 80 | | 16 | | 5 | |
|---|---|---|---|---|---|---|---|---|---|---|
| Mean (SD) | 57.95 | (28.724) | 70.36 | (42.897) | 61.98 | (34.207) | 53.81 | (26.074) | 63.58 | (30.071) |

Time between Angiography and First Infusion (h) ^{a}

| | N | 51 | | 26 | | 77 | | 15 | | 5 | |
|---|---|---|---|---|---|---|---|---|---|---|---|
| | Mean (SD) | 61.83 | (28.187) | 71.79 | (42.621) | 65.20 | (33.804) | 57.70 | (25.561) | 64.78 | (29.772) |
| 12-<24 | n(%) | 3 | (5.9) | 2 | (7.7) | 5 | (6.5) | 1 | (6.7) | 0 | |
| 24-<48 | n(%) | 18 | (35.3) | 7 | (26.9) | 25 | (32.5) | 6 | (40.0) | 1 | (20.0) |
| ≥48 | n(%) | 30 | (58.8) | 17 | (65.4) | 47 | (61.0) | 8 | (53.3) | 4 | (80.0) |

Time between Randomization and First Infusion (h)

| N | 52 | | 28 | | 80 | | 15 | | 6 | |
|---|---|---|---|---|---|---|---|---|---|---|
| Mean (SD) | 1.76 | (0.841) | 1.40 | (0.717) | 1.63 | (0.813) | 1.65 | (0.727) | 1.28 | (0.625) |

Time between Index MI and First Infusion (h)

| N | 52 | | 28 | | 80 | | 15 | | 6 | |
|---|---|---|---|---|---|---|---|---|---|---|
| Mean (SD) | 78.75 | (29.916) | 90.00 | (41.008) | 82.69 | (34.375) | 62.23 | (25.245) | 66.00 | (25.936) |

Time between Angiography and Local Lab for Eligibility (h) ^{a}

| | N | 54 | | 26 | | 80 | | 16 | | 5 | |
|---|---|---|---|---|---|---|---|---|---|---|---|
| | Mean (SD) | 51.78 | (28.516) | 62.87 | (42.894) | 55.38 | (33.987) | 47.99 | (27.115) | 54.94 | (30.684) |
| 12-<24 | n(%) | 14 | (25.9) | 6 | (23.1) | 20 | (25.0) | 4 | (25.0) | 0 | |
| 24-<48 | n(%) | 15 | (27.8) | 9 | (34.6) | 24 | (30.0) | 6 | (37.5) | 4 | (80.0) |
| ≥48 | n(%) | 25 | (46.3) | 11 | (42.3) | 36 | (45.0) | 6 | (37.5) | 1 | (20.0) |

| | | STEMI | | NSTEMI | | | | | |
|---|---|---|---|---|---|---|---|---|---|
| | | Total | | CSL112 6 g | | Placebo | | Total | |
| Timing Characteristics | Descriptive Statistic | (N = 83) | | (N = 55) | | (N = 28) | | (N = 83) | |
| Time between Index MI and Angiography (h) | | | | | | | | | |
| | N | 21 | | 38 | | 21 | | 59 | |
| | Mean (SD) | 4.07 | (5.997) | 21.74 | (17.966) | 24.73 | (20.290) | 22.80 | (18.707) |
| Time between Angiography and Randomization (h) | | | | | | | | | |
| | N | 21 | | 38 | | 21 | | 59 | |
| | Mean (SD) | 56.14 | (26.625) | 59.69 | (29.929) | 71.98 | (45.881) | 64.07 | (36.504) |
| Time between Angiography and First Infusion (h) ^{a} | | | | | | | | | |
| | N | 20 | | 36 | | 21 | | 57 | |
| | Mean (SD) | 59.47 | (26.037) | 63.55 | (29.383) | 73.46 | (45.587) | 67.20 | (36.125) |
| 12-<24 | n(%) | 1 | (5.0) | 2 | (5.6) | 2 | (9.5) | 4 | (7.0) |
| 24-<48 | n(%) | 7 | (35.0) | 12 | (33.3) | 6 | (28.6) | 18 | (31.6) |
| ≥48 | n(%) | 12 | (60.0) | 22 | (61.1) | 13 | (61.9) | 35 | (61.4) |
| Time between Randomization and First Infusion (h) | | | | | | | | | |
| | N | 21 | | 37 | | 22 | | 59 | |
| | Mean (SD) | 1.55 | (0.705) | 1.80 | (0.888) | 1.44 | (0.750) | 1.66 | (0.851) |
| Time between Index MI and First Infusion (h) | | | | | | | | | |
| | N | 21 | | 37 | | 22 | | 59 | |
| | Mean (SD) | 63.31 | (24.846) | 85.44 | (29.331) | 96.55 | (42.339) | 89.58 | (34.819) |
| Time between Angiography and Local Lab for Eligibility (h) ^{a} | | | | | | | | | |
| | N | 21 | | 38 | | 21 | | 59 | |
| | Mean (SD) | 49.64 | (27.366) | 53.38 | (29.290) | 64.76 | (45.739) | 57.43 | (36.040) |
| 12-<24 | n(%) | 4 | (19.0) | 10 | (26.3) | 6 | (28.6) | 16 | (27.1) |
| 24-<48 | n(%) | 10 | (47.6) | 9 | (23.7) | 5 | (23.8) | 14 | (23.7) |
| ≥48 | n(%) | 7 | (33.3) | 19 | (50.0) | 10 | (47.6) | 29 | (49.2) |

ITT = Intent to Treat,
MI = Myocardial Infarction,
NSTEMI = Non-ST-Elevation Myocardial Infarction,
SD = Standard Deviation,
STEMI = ST-Elevation Myocardial Infarction
^{a} Percentages are based on the number of subjects within the parent category.

TABLE 18
Summary of Co-primary Endpoints of Treatment-emergent Renal Serious
Adverse Events and Acute Kidney Injury Events (Safety Population)

| | | Number of | Rate difference between | |
|---|---|---|---|---|
| Co-primary | | subjects with | treatment groups | |
| endpoint | Number of | events n (%), | Difference | |
| Treatment | subjects, n | n′ | in rates | 95% CI^{a} |
| Renal SAEs | | | | |
| CSL112 6 g | 52 | 1 (1.9) 1 | −0.124 | (−0.296, −0.005) |
| (N = 52) | | | | |
| Placebo | 28 | 4 (14.3) 5 | NA | NA |
| (N = 28) | | | | |
| AKI Events | | | | |
| CSL112 6 g | 50 | 2 (4.0) 2 | −0.103 | (−0.277, 0.025) |
| (N = 52) | | | | |
| Placebo | 28 | 4 (14.3) 4 | NA | NA |
| (N = 28) | | | | |

AKI = Acute Kidney Injury,
CI = Confidence Interval,
NA = not applicable,
n (%) = counts the number and percentage of subjects that experienced an event,
n′ = counts the number of instances,
SAE = Serious Adverse Event
^{a}95% CIs of the difference in subject incidence rates were calculated using the Newcombe-Wilson score method intervals when at least 1 event occurs, or otherwise, with the exact, one-sided, upper 97.5% confidence intervals for the incidence rates in each of the treatment arms.

TABLE 19
Co-Primary Exploratory Summary of the Renal Safety Endpoint, by Subgroup (Safety Population)

| | Co-Primary Endpoint | | | |
|---|---|---|---|---|
| | Renal SAE | | AKI Events | |
| | Number of | Number of | Number of | Number of |
| Subgroup^{a} | Subjects | Subjects with | Subjects | Subjects with |
| Treatment | with Data, n | Events, n (%) Nn′ | with Data, n | Events, n (%) n′ |
| eGFR <30 mL/min/1.73 m² | | | | |
| CSL112 6 g (N = 52) | 3 | 0 | 3 | 0 |
| Placebo (N = 28) | 1 | 0 | 1 | 0 |
| eGFR 30-<45 mL/min/1.73 m² | | | | |
| CSL112 6 g (N = 52) | 18 | 0 | 18 | 0 |
| Placebo (N = 28) | 15 | 3 (20.0) 4 | 15 | 1 (6.7) 1 |
| eGFR 45 -<60 mL/min/1.73 m² | | | | |
| CSL112 6 g (N = 52) | 25 | 1 (4.0) 1 | 25 | 2 (8.0) 2 |
| Placebo (N = 28) | 11 | 1 (9.1) 1 | 11 | 2 (18.2) 2 |
| eGFR >=60 mL/min/1.73 m² | | | | |
| CSL112 6 g (N = 52) | 4 | 0 | 4 | 0 |
| Placebo (N = 28) | 1 | 0 | 1 | 1 (100) 1 |
| With Medical History of Diabetes | | | | |
| Requiring Current Treatment with Any | | | | |
| Anti-Diabetic Medication | | | | |
| CSL112 6 g (N = 52) | 22 | 0 | 22 | 0 |
| Placebo (N = 28) | 12 | 3 (25.0) 4 | 12 | 4 (33.3) 4 |
| Without Medical History of Diabetes | | | | |
| Requiring Current Treatment with Any | | | | |
| Anti-Diabetic Medication | | | | |
| CSL112 6 g (N = 52) | 30 | 1 (3.3) 1 | 28 | 2 (7.1) 2 |
| Placebo (N = 28) | 16 | 1 (6.3) 1 | 16 | 0 |

AKI = Acute Kidney Injury,
CKD-EPI = Chronic Kidney Disease Epidemiology Collaboration,
eGFR = estimated Glomerular Filtration Rate,
MedDRA = Medical Dictionary for Regulatory Activities,
PT = Preferred Term,
SAE = Serious Adverse Event,
SMQ = Standard MedDRA Query.
Percentages are based on the number of subjects with data.
n (%) counts the number and percentage of subjects that experienced an event. n′ counts the number of instances.
^{a}Renal function is based on calculated eGFR measurements as recorded in the central laboratory data, using the CKD-EPI equation.
Note:
The incidence rate was calculated using a denominator based on the number of subjects with data. Treatment-emergent was defined as occurring on or after the start of the first infusion.

TABLE 20
Overall Summary of Adverse Events (Safety Population)

| | Number (%) of Subjects | | |
|---|---|---|---|
| | CSL112 | | |
| | 6 g | Placebo | Total |
| | (N = 52) | (N = 28) | (N = 80) |
| Subjects with any TEAE | 38 (73.1) | 20 (71.4) | 58 (72.5) |
| Any Study-Treatment Related TEAE | 4 (7.7) | 1 (3.6) | 5 (6.3) |
| Subjects with any Serious TEAE | 12 (23.1) | 10 (35.7) | 22 (27.5) |
| Any Study-Treatment Related Serious | 0 | 1 (3.6) | 1 (1.3) |
| TEAE | | | |
| Any Fatal TEAE^{a} | 2 (3.8) | 2 (7.1) | 4 (5.0) |
| Any Study-Treatment Related Fatal | 0 | 0 | 0 |
| TEAE | | | |
| Any TEAE with CTCAE Grade ≥3 | 13 (25.0) | 10 (35.7) | 23 (28.8) |
| Any Treatment Emergent Potential | 0 | 0 | 0 |
| Hemolysis Events | | | |
| Any Treatment Emergent Bleeding | 7 (13.5) | 5 (17.9) | 12 (15.0) |
| Events | | | |
| Any Suspected Adverse Drug | 30 (57.7) | 4 (14.3) | 34 (42.5) |
| Reaction | | | |

CTCAE = Common Terminology Criteria for Adverse Events,
TEAE = treatment-emergent adverse event
^{a}For each treatment group 1 death due to unknown cause; 1 death due to heart failure.
Note:
Percentages are based on the number of subjects in the safety population for each treatment group.

TABLE 21
Treatment-Emergent Study Treatment-Related Adverse Events,
by Preferred Term (Safety Population)

| | Number (%) of Subjects | | |
|---|---|---|---|
| | CSL112 6 g | Placebo | Total |
| Preferred Term | (N = 52) | (N = 28) | (N = 80) |
| Subjects with any Study | 4 (7.7) | 1 (3.6) | 5 (6.3) |
| Treatment-Related TEAE | | | |
| Alanine aminotransferase increased | 1 (1.9) | 0 | 1 (1.3) |
| Blood bilirubin increased | 1 (1.9) | 0 | 1 (1.3) |
| Hyperventilation | 1 (1.9) | 0 | 1 (1.3) |
| Infusion site swelling | 1 (1.9) | 0 | 1 (1.3) |
| Renal failure | 0 | 1 (3.6) | 1 (1.3) |

MedDRA = Medical Dictionary for Regulatory Activities,
TEAE = Treatment Emergent Adverse Event.
Note:
Adverse events were coded to system organ class and preferred term using MedDRA version 20.0. Subjects may contribute to more than one preferred term, but only once within a preferred term. Percentages are based on the number of subjects in the safety population for each treatment group.

TABLE 22
Summary of Abnormal Serum Creatinine Values (Central Laboratory) During the Active Treatment Period (Safety Population)

| | | Number (%) of Subjects | | | | | | | | | | | |
|---|---|---|---|---|---|---|---|---|---|---|---|---|---|
| | | Central Laboratory Assessment | | | | | | Local Laboratory Assessment | | | | | |
| | | CSL112 6 g | | Placebo | | Total | | CSL112 6 g | | Placebo | | Total | |
| | | (N = 52) | | (N = 28) | | (N = 80) | | (N = 52) | | (N = 28) | | (N = 80) | |
| Any Stage 3 AKI (Central Laboratory) ^{a} | n (%) | 0 | | 0 | | 0 | | 0 | | 0 | | 0 | |
| Elevation in Serum Creatinine ≥3x | n (%) | 0 | | 0 | | 0 | | 0 | | 0 | | 0 | |
| the Baseline Value | | | | | | | | | | | | | |
| Serum Creatinine ≥4.0 mg/dL | n (%) | 0 | | 0 | | 0 | | 0 | | 0 | | 0 | |
| (353.6 μmol/L) | | | | | | | | | | | | | |
| Absolute Increase from Baseline, | ≤Baseline Value | 9 | (17.3) | 3 | (10.7) | 12 | (15.0) | 14 | (26.9) | 5 | (17.9) | 19 | (23.8) |
| Worst Case n (%) | | | | | | | | | | | | | |
| | >0 to <0.3 mg/dL | 35 | (67.3) | 18 | (64.3) | 53 | (66.3) | 30 | (57.7) | 13 | (46.4) | 43 | (53.8) |
| | ≥0.3 to ≤0.5 mg/dL | 4 | (7.7) | 4 | (14.3) | 8 | (10.0) | 5 | (9.6) | 6 | (21.4) | 11 | (13.8) |
| | >0.5 mg/dL | 2 | (3.8) | 2 | (7.1) | 4 | (5.0) | 3 | (5.8) | 3 | (10.7) | 6 | (7.5) |
| Absolute Increase from Baseline | ≥0.3 to ≤0.5 mg/dL | 1 | (1.9) | 4 | (14.3) | 5 | (6.3) | 2 | (3.8) | 3 | (10.7) | 5 | (6.3) |
| Sustained for ≥24 h, Worst | | | | | | | | | | | | | |
| Case n (%) | | | | | | | | | | | | | |
| | >0.5 mg/dL | 1 | (1.9) | 0 | | 1 | (1.3) | 2 | (3.8) | 0 | | 2 | (2.5) |
| Increases from Baseline, Worst | ≥1.5x Baseline | 1 | (1.9) | 1 | (3.6) | 2 | (2.5) | 2 | (3.8) | 2 | (7.1) | 4 | (5.0) |
| Case n (%) | ≥2x Baseline | 0 | | 0 | | 0 | | 1 | (1.9) | 0 | | 1 | (1.3) |
| | ≥3x Baseline | 0 | | 0 | | 0 | | 0 | | 0 | | 0 | |
| | ≥4.0 mg/dL | 0 | | 0 | | 0 | | 0 | | 0 | | 0 | |
| | (353.6 μmol/L) | | | | | | | | | | | | |
| Increases Sustained for ≥24 h, | ≥1.5x Baseline | 1 | (1.9) | 0 | | 1 | (1.3) | 1 | (1.9) | 0 | | 1 | (1.3) |
| Worst Case n (%) | ≥2x Baseline | 0 | | 0 | | 0 | | 0 | | 0 | | 0 | |
| | ≥3x Baseline | 0 | | 0 | | 0 | | 0 | | 0 | | 0 | |
| | ≥4.0 mg/dL | 0 | | 0 | | 0 | | 0 | | 0 | | 0 | |
| | (353.6 μmol/L) | | | | | | | | | | | | |
| Decrease in eGFR (Central) | n (%) | 5 | (9.6) | 4 | (14.3) | 9 | (11.3) | NA | | NA | | NA | |
| by ≥25% from Baseline ^{b} | | | | | | | | | | | | | |
| Decrease in eGFR (Central) | n (%) | 1 | (1.9) | 1 | (3.6) | 2 | (2.5) | NA | | NA | | NA | |
| by ≥25% from Baseline | | | | | | | | | | | | | |
| Sustained at Final Visit (Visit 8) ^{b} | | | | | | | | | | | | | |
| eGFR (Central) <30 mL/min/1.73 m² | n (%) | 0 | | 0 | | 0 | | NA | | NA | | NA | |
| at Final Visit (Visit 8) | | | | | | | | | | | | | |

AKI = Acute Kidney Injury, eGFR = estimated Glomerular Filtration Rate.
^{a} Defined as an elevation in serum creatinine during the Active Treatment Period to ≥3 × the baseline value or a serum creatinine of ≥4.0 mg/dL that was confirmed by repeat assessment using the central laboratory data.
^{b} Defined as a decrease of at least 25% starting during the Active Treatment Period.
Note:
The Active Treatment Period began at the time of a subject’s first infusion up until completion of Visit 7. In the absence of a Visit 7 assessment, the end of the Active Treatment Period was the date of the subject’s last administration of study medication + 10 days. Baseline assessment refers to the last assessment taken prior to the date/time of the start of first infusion of investigational product.

TABLE 23
Summary of Abnormal Liver Function Parameter Values (Regardless of
Confirmation) (Central Laboratory) During the Active Treatment Period (Safety
Population)

| | | | Number (%) of Subjects | | |
|---|---|---|---|---|---|
| | Number of | | CSL112 6 g | Placebo | Total |
| Laboratory Assessment | Subjects, n | Increase | (N = 52) | (N = 28) | (N = 80) |
| Active Treatment Period | | | | | |
| Worst Case^{a} | | | | | |
| Total or Direct Bilirubin^{c} | 79 | >1.5x ULN | 3 (5.8) | 0 | 3 (3.8) |
| | 79 | >2x ULN | 0 | 0 | 0 |
| Total Bilirubin | 79 | >1.5x ULN | 4 (7.7) | 1 (3.7) | 5 (6.3) |
| | 79 | >2x ULN | 1 (1.9) | 0 | 1 (1.3) |
| Direct Bilirubin | 79 | >1.5x ULN | 2 (3.8) | 0 | 2 (2.5) |
| | 79 | >2x ULN | 0 | 0 | 0 |
| ALT^{b} | 79 | >3x ULN | 0 | 0 | 0 |
| | 79 | >5x ULN | 0 | 0 | 0 |
| | 79 | >10x ULN | 0 | 0 | 0 |
| AST^{b} | 79 | >3x ULN | 1 (1.9) | 0 | 1 (1.3) |
| | 79 | >5x ULN | 1 (1.9) | 0 | 1 (1.3) |
| | 79 | >10x ULN | 0 | 0 | 0 |
| Concomitant elevations^{c} | 79 | Total or Direct Bilirubin | 0 | 0 | 0 |
| | | >2x, ALT >3x | | | |
| | 79 | Total or Direct Bilirubin | 0 | 0 | 0 |
| | | >2x, AST >3x | | | |
| Concomitant elevations | 79 | Total Bilirubin >2x, | 0 | 0 | 0 |
| | | ALT >3x | | | |
| | 79 | Total Bilirubin >2x, | 0 | 0 | 0 |
| | | AST >3x | | | |
| Concomitant elevations | 79 | Direct Bilirubin >2x, | 0 | 0 | 0 |
| | | ALT >3x | | | |
| | 79 | Direct Bilirubin >2x, | 0 | 0 | 0 |
| | | AST >3x | | | |

ALT = Alanine Aminotransferase,
AST = Aspartate Aminotransferase,
ULN = Upper Limit of Normal.
Percentages are based on the number of subjects with data.
All increases are summarized, regardless of confirmation by repeat assessment.
^{a}Summarizes the single worst value during the Active Treatment Period, including unscheduled assessments, for all subjects within the specified treatment group.
^{b}Increases relative to ULN range are sex specific.
^{c}For subjects with a history of Gilbert's Syndrome, direct bilirubin values are used in replacement for total bilirubin.
Note:
The Active Treatment Period began at the time of a subject's first infusion up until completion of Visit 7. In the absence of a Visit 7 assessment, the end of the Active Treatment Period was the date of the subject's last administration of study medication + 10 days. Visit 7 (7 to 10 days after last infusion) includes data for subjects who discontinued study treatment or withdrew from the study early (prior to Visit 7).

TABLE 24
Summary of Baseline-Corrected Pharmacokinetic Parameters (PK Population)

| Parameter | Treatment Group | Infusion | n | Mean | SD | Median | Q1, Q3 | Min, Max |
|---|---|---|---|---|---|---|---|---|
| ApoA-I | | | | | | | | |
| Cₘₐₓ (mg/dL) | CSL112 6 g (N = 52) | 1 | 52 | 124.6 | 25.38 | 127.0 | 112.0, 142.5 | 49, 188 |
| | | 4 | 38 | 141.5 | 41.11 | 147.5 | 127.0, 171.0 | −14, 213 |
| | Placebo (N = 28) | 1 | 28 | −4.5 | 9.46 | −2.0 | −9.5, 1.5 | −32, 9 |
| | | 4 | 21 | 1.4 | 23.57 | 0.0 | −12.0, 9.0 | −43, 66 |
| PC | | | | | | | | |
| Cₘₐₓ (mg/dL) | CSL112 6 g (N = 52) | 1 | 52 | 198.4 | 43.56 | 202.0 | 171.0, 229.0 | 80, 295 |
| | | 4 | 38 | 200.0 | 71.78 | 217.5 | 157.0, 248.0 | −34, 337 |
| | Placebo (N = 28) | 1 | 28 | −4.9 | 15.04 | −7.0 | −12.5, 4.5 | −43, 26 |
| | | 4 | 21 | −13.2 | 27.96 | −14.0 | −33.0, −3.0 | −66, 45 |

ApoA-I = Apolipoprotein A-I, Cₘₐₓ = Maximum Concentration, PC = Phosphatidylcholine, PK = pharmacokinetic, Q1 = 1^{st} Quartile, Q3 = 3^{rd} Quartile, SD = Standard Deviation.
Note:
Baseline-Corrected Values are calculated as (Visit Value − Baseline Value). Baseline assessment refers to the last assessment taken prior to the date/time of the start of first infusion of investigational product.

1. A method for increasing cholesterol efflux capacity (CEC) in a human after an acute myocardial infarction (MI) event, comprising:
within about seven (7) days of the acute MI event, administering to the patient a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol); and
subsequently administering the rHDL formulation to the human for at least about four (4) weeks;
thereby increasing cholesterol efflux capacity (CEC) in the human without causing a substantial alteration in liver and/or kidney function of the human.

2. A method for treating an acute myocardial infarction (MI) event in a human, comprising:
within about seven (7) days of the acute MI event, administering to the patient a reconstituted high density lipoprotein (rHDL) formulation an apolipoprotein or a fragment thereof, a lipid, a stabilizer and optionally a detergent, wherein the ratio between the apolipoprotein and the phospholipid is from about 1:20 to about 1:120 (mol:mol); and
subsequently administering the rHDL formulation to the human for at least about four (4) weeks;
thereby treating the acute myocardial infarction (MI) event in the human without causing a substantial alteration in liver and/or kidney function of the human.

3-4. (canceled)

5. The method of claim 1, wherein the subsequent administration of the rHDL formulation is weekly.

6. (canceled)

7. The method of claim 1, wherein total CEC is increased by 1.5-fold to 2.5 fold.

8. The method of claim 1, wherein ABCA1-dependent cholesterol efflux capacity is increased by about 3-fold to about 5-fold.

9. The method of claim 1, wherein the initial administration of the rHDL formulation is within five (5) days of the acute MI event.

10. The method of claim 1, wherein the initial administration of the rHDL formulation is no earlier than 12 hours after the acute MI event or after administration of a contrast agent for angiography.

11. The method of claim 1, wherein the rHDL formulation is intravenously (IV) infused.

12. The method of claim 11, wherein the rate of infusion is about 1-3 g apolipoprotein per hour.

13. The method of claim 1, wherein a significant alteration in liver function is measured as an alanine aminotransferase activity (ALT) of more than 3 times the upper limit of normal (ULN) and/or an increase in total bilirubin of at least 2×ULN.

14. The method of claim 1, wherein a significant alteration in kidney function is measured as serum creatinine at least 1.5 times the baseline value and/or a requirement for renal replacement therapy.

15. The method of claim 1, wherein a significant alteration in kidney function is measured as an estimated glomerular filtration rate (eGFR) of less than 60 mL/min/m².

16-30. (canceled)

31. The method of claim 1, wherein the rHDL formulation comprises at least 6 g of an apoA-I, phosphatidylcholine, a stabilizer and sodium cholate at a level selected from the group consisting of about 0.5-1.5 g/L and/or about 0.010-0.030 g/g apoA-I, and from about 1.0% to less than 6.0% w/w of sucrose, wherein the ratio between the apoA-I and the phosphatidylcholine is from about 1:20 to about 1:120 (mol:mol),
wherein a substantial alteration in liver function is an ALT of more than about 2 or 3 times the upper limit of normal (ULN) or an increase in total bilirubin of at least 1.5 to 2 times ULN; and
a substantial alteration in kidney function is a serum creatinine greater than or equal to about 1.2-1.5 times the baseline value and/or an eGFR substantially less than 90 mL/min/m².

32. (canceled)

33. A method for reducing the risk of a major adverse cardiac event (MACE) in a human patient with moderate renal impairment (Mod RI) who has not previously experienced an MI event, or who has not experienced an MI event within seven days prior to starting treatment, comprising:
administering to the patient a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol),
thereby reducing the risk of a MACE in the patient.

34. A method for increasing CEC in a human patient with moderate renal impairment (Mod RI) who has not previously experienced an MI event, or who has not experienced an MI event within seven days prior to starting treatment, comprising:
administering to the patient a reconstituted high density lipoprotein (rHDL) formulation comprising an apolipoprotein or a fragment thereof, a lipid, a stabilizer and optionally a detergent, wherein the ratio between the apolipoprotein and the lipid is from about 1:20 to about 1:120 (mol:mol),
thereby increasing cholesterol efflux capacity (CEC).

35-60. (canceled)

61. The method of claim 1, further comprising administering one or more therapeutic agents that assist or facilitate treatment, prevention or reduction in risk of an acute myocardial infarction (MI) event and/or MACE and/or increasing cholesterol efflux capacity (CEC) in a human patient selected from one or more lipid-modifying agents; one or more cholesterol absorption inhibitors: one or more anti-coagulants: one or more anti-hypertensive agents; and one or more bile acid binding molecules.

62-71. (canceled)

72. The method of claim 1, wherein the human patient has moderate renal impairment (Mod RI).
